(19) United States
(12) Patent Application Publication
(10) Pub. No.: US 2023/0041030 A1
(43) Pub. Date: Feb. 9, 2023

(54) ANTIGEN-BINDING PROTEINS TARGETING SHARED NEOANTIGENS

(71) Applicant: Gritstone bio, Inc., Emeryville, CA (US)

(72) Inventors: Karin Jooss, Emeryville, CA (US); Aleksandra Katarzyna Nowicka, Watertown, MA (US); Abubakar Jalloh, Somerville, MA (US); Roman Yelensky, Newton, MA (US); James Xin Sun, Newton, MA (US); Jennifer Busby, Burlington, MA (US); Matthew Joseph Davis, Scituate, MA (US)

(21) Appl. No.: 17/744,354

(22) Filed: May 13, 2022

Related U.S. Application Data
(63) Continuation of application No. PCT/US2020/060605, filed on Nov. 13, 2020.
(60) Provisional application No. 63/030,774, filed on May 27, 2020.
(60) Provisional application No. 62/936,303, filed on Nov. 15, 2019.

(51) Int. Cl. C07K 16/28; C12N 5/0783; C12N 15/85; A61P 35/00

(52) U.S. Cl. CPC C07K 16/2833; C12N 5/0636; C12N 15/85; A61P 35/00; C07K 2317/565

(57) ABSTRACT

Provided herein are target HLA-PEPTIDE antigens, e.g., HLA-PEPTIDE neoantigens and shared tumor HLA-PEPTIDE antigens, and antigen binding proteins (ABPs) that bind the target HLA-PEPTIDE antigens. Also disclosed are methods for identifying target HLA-PEPTIDE antigens as well as identifying one or more antigen binding proteins that bind a given HLA-PEPTIDE target antigen.

### RELATED APPLICATIONS

This application is continuation of International Application No. PCT/US20/60605, filed Nov. 13, 2020, which claims the benefit of U.S. Provisional Application Nos: 62/936,303 filed Nov. 15, 2019 and 63/030,774 filed May 27, 2020, each of which is hereby incorporated in its entirety by reference for all purposes.

### SEQUENCE LISTING

The instant application contains a Sequence Listing which has been filed electronically in ASCII format and is hereby incorporated by reference in its entirety. Said ASCII copy, created on Nov. 13, 2020, is named GSO-080WO_SL.txt and is 6,925,625 bytes in size.

### BACKGROUND

It is recognized that MHCs display intracellularly processed protein fragments on the cell surface. In humans, MHC is referred to as human leukocyte antigen or HLA. In particular, MHC class I molecules are expressed on the surface of virtually all nucleated cells in the body. They are dimeric molecules comprising a transmembrane heavy chain, comprising the peptide antigen binding cleft, and a smaller extracellular chain termed beta2-microglobulin. MHC class I molecules present peptides derived from the degradation of cytosolic proteins by the proteasome, a multi-unit structure in the cytoplasm, (Niedermann G., 2002. Curr Top Microbiol Immunol. 268:91-136; for processing of bacterial antigens, refer to Wick M J, and Ljunggren H G., 1999. Immunol Rev. 172:153-62). Cleaved peptides are transported into the lumen of the endoplasmic reticulum (ER) by the transporter associated with antigen processing (TAP) where they are bound to the groove of the assembled class I molecule, and the resultant MHC/peptide complex is transported to the cell membrane to enable antigen presentation to T lymphocytes (Yewdell J W., 2001. Trends Cell Biol. 11:294-7; Yewdell J W. and Bennink J R., 2001. Curr Opin Immunol. 13:13-8). Alternatively, cleaved peptides can be loaded onto MHC class I molecules in a TAP-independent manner and can also present extracellularly-derived proteins through a process of cross-presentation.

MHC genes are highly polymorphic across species populations, comprising multiple common alleles for each individual gene. As such, a given MHC allele/peptide complex comprising a specific HLA subtype and a specific peptide fragment presents a novel protein structure on the cell surface that can be targeted by a novel antigen-binding protein (e.g., TCRs or antigen binding fragments thereof). However, such TCR-based approaches first require the identification of the complex's structure (peptide sequence and MHC subtype).

Tumor cells can express neoantigens and may display such antigens on the surface of the tumor cell via MHC presentation. Such tumor-associated neoantigens, comprising the novel protein structure formed by the peptide-MHC subtype complex, can be used for development of novel immunotherapeutic reagents for the specific targeting of tumor cells. For example, tumor-associated antigens can be used to identify therapeutic antigen binding proteins, e.g., TCRs, or antigen-binding fragments thereof. However, accurate identification of such neoantigens has been challenging.

Initial methods have been proposed incorporating mutation-based analysis using next-generation sequencing, RNA gene expression, and prediction of MHC binding affinity of candidate neoantigen peptides⁸. However, these proposed methods can fail to model the entirety of the epitope generation process, which contains many steps (e.g., TAP transport, proteasomal cleavage, and/or TCR recognition) in addition to gene expression and MHC binding⁹. Consequently, existing methods are likely to suffer from reduced low positive predictive value (PPV).

Indeed, analyses of peptides presented by tumor cells performed by multiple groups have shown that <5% of peptides predicted to be presented using gene expression and MHC binding affinity are actually found on the tumor surface MHC^{10,11}. This low correlation between binding predicted and actual MHC presentation was further reinforced by recent observations of the lack of predictive accuracy improvement of binding-restricted neoantigens for checkpoint inhibitor response over the number of mutations alone.¹²

This low positive predictive value (PPV) of existing methods for predicting presentation presents a problem for neoantigen-based immunotherapy design. If immunotherapies are designed using predictions with a low PPV, many of them will be clinically ineffective.

Accordingly, there is a need for the discovery and identification of tumor-associated HLA-peptide complexes with high positive predictive value, and a need for the development of TCR-based immunotherapies targeting such complexes.

### SUMMARY

Provided herein is an antigen binding protein (ABP) that specifically binds to an HLA-PEPTIDE antigen comprising an HLA-restricted peptide complexed with an HLA Class I molecule, wherein the HLA-restricted peptide is located in the peptide binding groove of an α1/α2 heterodimer portion of the HLA Class I molecule, wherein the HLA Class I molecule and the HLA-restricted peptide are each selected from an HLA-PEPTIDE antigen as described in any one of SEQ ID NOs:10,755 to 29,364, and wherein the ABP comprises a T cell receptor (TCR) or antigen-binding fragment thereof.

In some aspects, the HLA-restricted peptide is between about 5-15 amino acids in length. In some aspects, the HLA-restricted peptide is between about 8-12 amino acids in length, optionally 8, 9, 10, 11, or 12 amino acids in length.

In some aspects, the HLA-PEPTIDE antigen is selected from the group consisting of a RAS_G12D MHC Class I antigen comprising HLA-A*11:01 and the restricted peptide VVVGADGVGK; a RAS_G12V MHC Class I antigen comprising HLA-A*11:01 and the restricted peptide VVVGAVGVGK; a RAS_G12C MHC Class I antigen comprising HLA-A*02:01 and the restricted peptide KLVVVGACGV; a CTNNB1_S45P MHC Class I antigen comprising HLA-A*03:01 and the restricted peptide TTAPPLSGK; a RAS_G12D MHC Class I antigen comprising HLA-A*11:01 and the restricted peptide VVGADGVGK; a RAS_G12V MHC Class I antigen comprising HLA-A*11:01 and the restricted peptide VVGAVGVGK; a RAS_G12V MHC Class I antigen comprising HLA-C*01:02 and the restricted peptide AVGVGKSAL; a RAS_G12V MHC Class I antigen comprising HLA-A*03:01 and the restricted peptide VVVGAVGVGK; a TP53_K132N MHC Class I antigen comprising HLA-A*24:02 and the restricted peptide TYSPALNNMF; a CTNNB1_S37Y MHC Class I antigen comprising HLA-A*02:01 and the restricted peptide YLDSGIHYGA; a RAS_G12C MHC Class I antigen comprising HLA-A*03:01 and the restricted peptide VVVGACGVGK; a RAS_G12C MHC Class I antigen comprising HLA-A*11:01 and the restricted peptide VVVGACGVGK; a RAS_G12D MHC Class I antigen comprising HLA-A*03:01 and the restricted peptide VVVGADGVGK; a RAS_Q61H MHC Class I antigen comprising HLA-A*01:01 and the restricted peptide ILDTAGHEEY; and a TP53_R213L MHC Class I antigen comprising A*02:01 and the restricted peptide YLDDRNTFL.

In some aspects, the restricted peptide comprises a RAS_G12A mutation, and wherein the HLA Class I molecule is HLA-A*02:01; the restricted peptide comprises a RAS_G12A mutation, and wherein the HLA Class I molecule is HLA-A*02:06; the restricted peptide comprises a RAS_G12A mutation, and wherein the HLA Class I molecule is HLA-B*27:05; the restricted peptide comprises a RAS_G12A mutation, and wherein the HLA Class I molecule is HLA-B*35:01; the restricted peptide comprises a RAS_G12A mutation, and wherein the HLA Class I molecule is HLA-B*41:02; the restricted peptide comprises a RAS_G12A mutation, and wherein the HLA Class I molecule is HLA-B*48:01; the restricted peptide comprises a RAS_G12A mutation, and wherein the HLA Class I molecule is HLA-C*08:03; the restricted peptide comprises a RAS_G12C mutation, and wherein the HLA Class I molecule is HLA-A*02:01; the restricted peptide comprises a RAS_G12C mutation, and wherein the HLA Class I molecule is HLA-A*02:01; the restricted peptide comprises a RAS_G12C mutation, and wherein the HLA Class I molecule is HLA-A*03:02; the restricted peptide comprises a RAS_G12C mutation, and wherein the HLA Class I molecule is HLA-A*68:01; the restricted peptide comprises a RAS_G12C mutation, and wherein the HLA Class I molecule is HLA-B*27:05; the restricted peptide comprises a RAS_G12D mutation, and wherein the HLA Class I molecule is HLA-A*02:01; the restricted peptide comprises a RAS_G12D mutation, and wherein the HLA Class I molecule is HLA-A*02:05; the restricted peptide comprises a RAS_G12D mutation, and wherein the HLA Class I molecule is HLA-A*03:01; the restricted peptide comprises a RAS_G12D mutation, and wherein the HLA Class I molecule is HLA-A*11:01; the restricted peptide comprises a RAS_G12D mutation, and wherein the HLA Class I molecule is HLA-A*11:01; the restricted peptide comprises a RAS_G12D mutation, and wherein the HLA Class I molecule is HLA-A*26:01; the restricted peptide comprises a RAS_G12D mutation, and wherein the HLA Class I molecule is HLA-A*31:01; the restricted peptide comprises a RAS_G12D mutation, and wherein the HLA Class I molecule is HLA-A*68:01; the restricted peptide comprises a RAS_G12D mutation, and wherein the HLA Class I molecule is HLA-B*07:02; the restricted peptide comprises a RAS_G12D mutation, and wherein the HLA Class I molecule is HLA-B*08:01; the restricted peptide comprises a RAS_G12D mutation, and wherein the HLA Class I molecule is HLA-B*13:02; the restricted peptide comprises a RAS_G12D mutation, and wherein the HLA Class I molecule is HLA-B*15:01; the restricted peptide comprises a RAS_G12D mutation, and wherein the HLA Class I molecule is HLA-B*27:05; the restricted peptide comprises a RAS_G12D mutation, and wherein the HLA Class I molecule is HLA-B*35:01; the restricted peptide comprises a RAS_G12D mutation, and wherein the HLA Class I molecule is HLA-B*37:01; the restricted peptide comprises a RAS_G12D mutation, and wherein the HLA Class I molecule is HLA-B*38:01; the restricted peptide comprises a RAS_G12D mutation, and wherein the HLA Class I molecule is HLA-B*40:01; the restricted peptide comprises a RAS_G12D mutation, and wherein the HLA Class I molecule is HLA-B*40:02; the restricted peptide comprises a RAS_G12D mutation, and wherein the HLA Class I molecule is HLA-B*44:02; the restricted peptide comprises a RAS_G12D mutation, and wherein the HLA Class I molecule is HLA-B*44:03; the restricted peptide comprises a RAS_G12D mutation, and wherein the HLA Class I molecule is HLA-B*48:01; the restricted peptide comprises a RAS_G12D mutation, and wherein the HLA Class I molecule is HLA-B*50:01; the restricted peptide comprises a RAS_G12D mutation, and wherein the HLA Class I molecule is HLA-B*57:01; the restricted peptide comprises a RAS_G12D mutation, and wherein the HLA Class I molecule is HLA-C*01:02; the restricted peptide comprises a RAS_G12D mutation, and wherein the HLA Class I molecule is HLA-C*02:02; the restricted peptide comprises a RAS_G12D mutation, and wherein the HLA Class I molecule is HLA-C*03:03; the restricted peptide comprises a RAS_G12D mutation, and wherein the HLA Class I molecule is HLA-C*03:04; the restricted peptide comprises a RAS_G12D mutation, and wherein the HLA Class I molecule is HLA-C*04:01; the restricted peptide comprises a RAS_G12D mutation, and wherein the HLA Class I molecule is HLA-C*05:01; the restricted peptide comprises a RAS_G12D mutation, and wherein the HLA Class I molecule is HLA-C*07:04; the restricted peptide comprises a RAS_G12D mutation, and wherein the HLA Class I molecule is HLA-C*08:02; the restricted peptide comprises a RAS_G12D mutation, and wherein the HLA Class I molecule is HLA-C*08:03; the restricted peptide comprises a RAS_G12D mutation, and wherein the HLA Class I molecule is HLA-C*16:01; the restricted peptide comprises a RAS_G12D mutation, and wherein the HLA Class I molecule is HLA-C*17:01; the restricted peptide comprises a RAS_G12D mutation, and wherein the HLA Class I molecule is HLA-B*41:02; the restricted peptide comprises a RAS_G12D mutation, and wherein the HLA Class I molecule is HLA-C*07:04; the restricted peptide comprises a RAS_G12V mutation, and wherein the HLA Class I molecule is HLA-A*02:01; the restricted peptide comprises a RAS_G12V mutation, and wherein the HLA Class I molecule is HLA-A*02:05; the restricted peptide comprises a RAS_G12V mutation, and wherein the HLA Class I molecule is HLA-A*02:06; the restricted peptide comprises a RAS_G12V mutation, and wherein the HLA Class I molecule is HLA-A*03:01; the restricted peptide comprises a RAS_G12V mutation, and wherein the HLA Class I molecule is HLA-A*03:01; the restricted peptide comprises a RAS_G12V mutation, and wherein the HLA Class I molecule is HLA-A*11:01; the restricted peptide comprises a RAS_G12V mutation, and wherein the HLA Class I molecule is HLA-A*11:01; the restricted peptide comprises a RAS_G12V mutation, and wherein the HLA Class I molecule is HLA-A*25:01; the restricted peptide comprises a RAS_G12V mutation, and wherein the HLA Class I molecule is HLA-A*26:01; the restricted peptide comprises a RAS_G12V mutation, and wherein the HLA Class I molecule is HLA-A*30:01; the restricted peptide comprises a RAS_G12V mutation, and wherein the HLA Class I molecule is HLA-A*31:01; the restricted peptide comprises a RAS_G12V mutation, and wherein the HLA Class I molecule is HLA-A*31:01; the restricted peptide comprises a RAS_G12V mutation, and wherein the HLA Class I molecule is HLA-A*32:01; the restricted peptide comprises a RAS_G12V mutation, and wherein the HLA Class I molecule is HLA-A*68:02; the restricted peptide comprises a RAS_G12V mutation, and wherein the HLA Class I molecule is HLA-B*07:02; the restricted peptide comprises a RAS_G12V mutation, and wherein the HLA Class I molecule is HLA-B*08:01; the restricted peptide comprises a RAS_G12V mutation, and wherein the HLA Class I molecule is HLA-B*13:02; the restricted peptide comprises a RAS_G12V mutation, and wherein the HLA Class I molecule is HLA-B*14:02; the restricted peptide comprises a RAS_G12V mutation, and wherein the HLA Class I molecule is HLA-B*15:01; the restricted peptide comprises a RAS_G12V mutation, and wherein the HLA Class I molecule is HLA-B*27:05; the restricted peptide comprises a RAS_G12V mutation, and wherein the HLA Class I molecule is HLA-B*39:01; the restricted peptide comprises a RAS_G12V mutation, and wherein the HLA Class I molecule is HLA-B*40:01; the restricted peptide comprises a RAS_G12V mutation, and wherein the HLA Class I molecule is HLA-B*40:02; the restricted peptide comprises a RAS_G12V mutation, and wherein the HLA Class I molecule is HLA-B*41:02; the restricted peptide comprises a RAS_G12V mutation, and wherein the HLA Class I molecule is HLA-B*44:05; the restricted peptide comprises a RAS_G12V mutation, and wherein the HLA Class I molecule is HLA-B*50:01; the restricted peptide comprises a RAS_G12V mutation, and wherein the HLA Class I molecule is HLA-B*51:01; the restricted peptide comprises a RAS_G12V mutation, and wherein the HLA Class I molecule is HLA-C*01:02; the restricted peptide comprises a RAS_G12V mutation, and wherein the HLA Class I molecule is HLA-C*01:02; the restricted peptide comprises a RAS_G12V mutation, and wherein the HLA Class I molecule is HLA-C*03:03; the restricted peptide comprises a RAS_G12V mutation, and wherein the HLA Class I molecule is HLA-C*03:04; the restricted peptide comprises a RAS_G12V mutation, and wherein the HLA Class I molecule is HLA-C*08:02; the restricted peptide comprises a RAS_G12V mutation, and wherein the HLA Class I molecule is HLA-C*14:02; the restricted peptide comprises a RAS_G12V mutation, and wherein the HLA Class I molecule is HLA-C*17:01; the restricted peptide comprises a KRAS_G13D mutation, and wherein the HLA Class I molecule is HLA-A*02:01; the restricted peptide comprises a KRAS_G13D mutation, and wherein the HLA Class I molecule is HLA-B*07:02; the restricted peptide comprises a KRAS_G13D mutation, and wherein the HLA Class I molecule is HLA-B*08:01; the restricted peptide comprises a KRAS_G13D mutation, and wherein the HLA Class I molecule is HLA-B*35:01; the restricted peptide comprises a KRAS_G13D mutation, and wherein the HLA Class I molecule is HLA-B*35:03; the restricted peptide comprises a KRAS_G13D mutation, and wherein the HLA Class I molecule is HLA-B*35:08; the restricted peptide comprises a KRAS_G13D mutation, and wherein the HLA Class I molecule is HLA-B*38:01; the restricted peptide comprises a KRAS_G13D mutation, and wherein the HLA Class I molecule is HLA-C*04:01; the restricted peptide comprises a KRAS_Q61H mutation, and wherein the HLA Class I molecule is HLA-A*01:01; the restricted peptide comprises a KRAS_Q61H mutation, and wherein the HLA Class I molecule is HLA-A*02:01; the restricted peptide comprises a KRAS_Q61H mutation, and wherein the HLA Class I molecule is HLA-A*23:01; the restricted peptide comprises a KRAS_Q61H mutation, and wherein the HLA Class I molecule is HLA-A*29:01; the restricted peptide comprises a KRAS_Q61H mutation, and wherein the HLA Class I molecule is HLA-A*30:02; the restricted peptide comprises a KRAS_Q61H mutation, and wherein the HLA Class I molecule is HLA-A*33:01; the restricted peptide comprises a KRAS_Q61H mutation, and wherein the HLA Class I molecule is HLA-A*68:01; the restricted peptide comprises a KRAS_Q61H mutation, and wherein the HLA Class I molecule is HLA-B*07:02; the restricted peptide comprises a KRAS_Q61H mutation, and wherein the HLA Class I molecule is HLA-B*08:01; the restricted peptide comprises a KRAS_Q61H mutation, and wherein the HLA Class I molecule is HLA-B*18:01; the restricted peptide comprises a KRAS_Q61H mutation, and wherein the HLA Class I molecule is HLA-B*35:01; the restricted peptide comprises a KRAS_Q61H mutation, and wherein the HLA Class I molecule is HLA-B*38:01; the restricted peptide comprises a KRAS_Q61H mutation, and wherein the HLA Class I molecule is HLA-B*40:01; the restricted peptide comprises a KRAS_Q61H mutation, and wherein the HLA Class I molecule is HLA-B*44:02; the restricted peptide comprises a KRAS_Q61H mutation, and wherein the HLA Class I molecule is HLA-C*03:04; the restricted peptide comprises a KRAS_Q61H mutation, and wherein the HLA Class I molecule is HLA-C*05:01; or the restricted peptide comprises a KRAS_Q61H mutation, and wherein the HLA Class I molecule is HLA-C*08:02.

In some aspects, the restricted peptide comprises a KRAS_G13D mutation, and wherein the HLA Class I molecule is C*08:02 or A*11:01; the restricted peptide comprises a KRAS_Q61K mutation, and wherein the HLA Class I molecule is A*01:01; the restricted peptide comprises a NRAS_Q61K mutation, and wherein the HLA Class I molecule is A*01:01; the restricted peptide comprises a TP53_R249M mutation, and wherein the HLA Class I molecule is B*35:12, B*35:03, or B*35:01; the restricted peptide comprises a CTNNB1_S45P mutation, and wherein the HLA Class I molecule is A*03:01, A*11:01, A*68:01, or A*03:02; the restricted peptide comprises a CTNNB1_S45F mutation, and wherein the HLA Class I molecule is A*03:01, A*11:01, or A*68:01; the restricted peptide comprises a ERBB2_Y772_A775dup mutation, and wherein the HLA Class I molecule is B*18:01; the restricted peptide comprises a KRAS_G12D mutation, and wherein the HLA Class I molecule is A*11:01, A*03:01, or C*08:02; the restricted peptide comprises a NRAS_G12D mutation, and wherein the HLA Class I molecule is A*11:01, A*03:01, or C*08:02; the restricted peptide comprises a KRAS_Q61D mutation, and wherein the HLA Class I molecule is A*01:01; the restricted peptide comprises a NRAS_Q61D mutation, and wherein the HLA Class I molecule is A*01:01; the restricted peptide comprises a CTNNB1_T41A mutation, and wherein the HLA Class I molecule is A*03:01, A*03:02, A*11:01, B*15:10, C*03:03, or C*03:04; the restricted peptide comprises a TP53_K132N mutation, and wherein the HLA Class I molecule is A*24:02 or A*23:01; the restricted peptide comprises a KRAS_G12A mutation, and wherein the HLA Class I molecule is A*03:01 or A*11:01; the restricted peptide comprises a KRAS_Q61L mutation, and wherein the HLA Class I molecule is A*01:01; the restricted peptide comprises a NRAS_Q61L mutation, and wherein the HLA Class I molecule is A*01:01; the restricted peptide comprises a TP53_R213L mutation, and wherein the HLA Class I molecule is A*02:07, C*08:02, or A*02:01; the restricted peptide comprises a BRAF_G466V mutation, and wherein the HLA Class I molecule is B*15:01, or B*15:03; the restricted peptide comprises a KRAS_G12V mutation, and wherein the HLA Class I molecule is A*03:01, A*03:02, A*11:01, or C*01:02; the restricted peptide comprises a KRAS_Q61H mutation, and wherein the HLA Class I molecule is A*01:01; the restricted peptide comprises a NRAS_Q61H mutation, and wherein the HLA Class I molecule is A*01:01; the restricted peptide comprises a CTNNB1_S37F mutation, and wherein the HLA Class I molecule is A*01:01, A*23:01, A*24:02, B*15:10, B*39:06, C*05:01, C*14:02, or C*14:03; the restricted peptide comprises a TP53_S127Y mutation, and wherein the HLA Class I molecule is A*11:01 or A*03:01; the restricted peptide comprises a TP53_K132E mutation, and wherein the HLA Class I molecule is A*24:02, C*14:03, or A*23:01; the restricted peptide comprises a KRAS_G12C mutation, and wherein the HLA Class I molecule is A*02:01, A*11:01, or A*03:01; the restricted peptide comprises a NRAS_G12C mutation, and wherein the HLA Class I molecule is A*02:01, A*11:01, or A*03:01; the restricted peptide comprises a EGFR_L858D mutation, and wherein the HLA Class I molecule is A*11:01, or A*03:01; the restricted peptide comprises a TP53_Y220C mutation, and wherein the HLA Class I molecule is A*02:01; or the restricted peptide comprises a TP53_R175H mutation, and wherein the HLA Class I molecule is A*02:01.

In some aspects, the HLA-PEPTIDE antigen is selected from: a CTNNB1_S45P MHC Class I antigen comprising A*11:01 and the restricted peptide TTAPPLSGK; a CTNNB1_T41AMHC Class I antigen comprising A*11:01 and the restricted peptide ATAPSLSGK; a RAS_G12D MHC Class I antigen comprising A*11:01 and the restricted peptide VVVGADGVGK; a RAS_G12V MHC Class I antigen comprising A*03:01 and the restricted peptide VVGAVGVGK; a RAS_G12V MHC Class I antigen comprising A*03:01 and the restricted peptide VVVGAVGVGK; a RAS_G12V MHC Class I antigen comprising A*11:01 and the restricted peptide VVGAVGVGK; a RAS_G12V MHC Class I antigen comprising A*11:01 and the restricted peptide VVVGAVGVGK; a KRAS_Q61R MHC Class I antigen comprising A*01:01 and the restricted peptide ILDTAGREEY; and a TP53_R213L MHC Class I antigen comprising A*02:01 and the restricted peptide YLDDRNTFL.

In some aspects, the HLA-restricted peptide comprises a RAS G12 mutation. In some aspects, the G12 mutation is a G12C, a G12D, a G12V, or a G12A mutation. In some aspects, the HLA-PEPTIDE antigen comprises an HLA Class I molecule selected from HLA-A*02:01, HLA-A*11:01, HLA-A*31:01, HLA-C*01:02, and HLA-A*03:01. In some aspects, the RAS G12 mutation is any one or more of: a KRAS, NRAS, and HRAS mutation. In some aspects, the HLA-PEPTIDE antigen is selected from: a RAS_G12C MHC Class I antigen comprising HLA-A*02:01 and the restricted peptide KLVVVGACGV; a RAS_G12C MHC Class I antigen comprising HLA-A*03:01 and the restricted peptide VVVGACGVGK; a RAS_G12C MHC Class I antigen comprising HLA-A*11:01 and the restricted peptide VVVGACGVGK; a RAS_G12D MHC Class I antigen comprising HLA-A*11:01 and the restricted peptide VVVGADGVGK; a RAS_G12D MHC Class I antigen comprising HLA-A*11:01 and the restricted peptide VVGADGVGK; a RAS_G12D MHC Class I antigen comprising HLA-A*03:01 and the restricted peptide VVVGADGVGK; a RAS_G12V MHC Class I antigen comprising HLA-A*11:01 and the restricted peptide VVVGAVGVGK; a RAS_G12V MHC Class I antigen comprising HLA-A*31:01 and the restricted peptide VVVGAVGVGK; a RAS_G12V MHC Class I antigen comprising HLA-A*11:01 and the restricted peptide VVGAVGVGK; a RAS_G12V MHC Class I antigen comprising HLA-C*01:02 and the restricted peptide AVGVGKSAL; and a RAS_G12V MHC Class I antigen comprising HLA-A*03:01 and the restricted peptide VVVGAVGVGK. In some aspects, the HLA-PEPTIDE antigen is selected from: a RAS_G12C MHC Class I antigen comprising HLA-A*02:01 and the restricted peptide KLVVVGACGV; a RAS_G12D MHC Class I antigen comprising HLA-A*11:01 and the restricted peptide VVVGADGVGK; a RAS_G12D MHC Class I antigen comprising HLA-A*11:01 and the restricted peptide VVGADGVGK; a RAS_G12V MHC Class I antigen comprising HLA-A*11:01 and the restricted peptide VVVGAVGVGK; a RAS_G12V MHC Class I antigen comprising HLA-A*31:01 and the restricted peptide VVVGAVGVGK; a RAS_G12V MHC Class I antigen comprising HLA-A*11:01 and the restricted peptide VVGAVGVGK; a RAS_G12V MHC Class I antigen comprising HLA-C*01:02 and the restricted peptide AVGVGKSAL; and a RAS_G12V MHC Class I antigen comprising HLA-A*03:01 and the restricted peptide VVVGAVGVGK. In some aspects, the HLA-PEPTIDE antigen is selected from: a RAS_G12C MHC Class I antigen comprising HLA-A*02:01 and the restricted peptide KLVVVGACGV; a RAS_G12D MHC Class I antigen comprising HLA-A*11:01 and the restricted peptide VVVGADGVGK; and a RAS_G12V MHC Class I antigen comprising HLA-A*11:01 and the restricted peptide VVVGAVGVGK. In some aspects, the HLA-PEPTIDE antigen is a RAS_G12C MHC Class I antigen comprising HLA-A*02:01 and the restricted peptide KLVVVGACGV. In some aspects, the HLA-PEPTIDE antigen is a RAS_G12D MHC Class I antigen comprising HLA-A*11:01 and the restricted peptide VVVGADGVGK. In some aspects, the HLA-PEPTIDE antigen is a RAS_G12V MHC Class I antigen comprising HLA-A*11:01 and the restricted peptide VVVGAVGVGK.

In some aspects, the HLA-restricted peptide comprises a RAS Q61 mutation. In some aspects, the Q61 mutation is a Q61H, a Q61K, a Q61R, or a Q61L mutation. In some aspects, the HLA-PEPTIDE antigen is a RAS_Q61H MHC Class I antigen comprising HLA-A*01:01 and the restricted peptide ILDTAGHEEY.

In some aspects, the HLA-restricted peptide comprises a TP53 mutation. In some aspects, the TP53 mutation comprises a R213L, S127Y, Y220C, R175H, or R249M mutation. In some aspects, the HLA-PEPTIDE antigen is a TP53 R213L MHC Class I antigen comprising A*02:01 and the restricted peptide YLDDRNTFL.

In some aspects, the antigen binding protein binds to the HLA-PEPTIDE antigen through at least one contact point with the HLA Class I molecule and through at least one contact point with the HLA-restricted peptide.

In some aspects, the antigen binding protein binds to a RAS_G12C MHC Class I antigen comprising HLA-A*02:01 and the restricted peptide KLVVVGACGV, and wherein the ABP binds to the RAS_G12C MHC Class I antigen at a higher affinity than an HLA-PEPTIDE antigen comprising a different RAS G12 mutation. In some aspects, the ABP binds to the RAS_G12C MHC Class I antigen at a higher affinity than an HLA-PEPTIDE antigen comprising the restricted peptide KLVVVGAVGV and an HLA-A2 molecule In some aspects, the ABP does not bind to an HLA-PEPTIDE antigen comprising the restricted peptide KLVVVGAVGV and an HLA-A2 molecule.

In some aspects, the antigen binding protein is linked to a scaffold, optionally wherein the scaffold comprises serum albumin or Fc, optionally wherein Fc is human Fc and is an IgG (IgG1, IgG2, IgG3, IgG4), an IgA (IgA1, IgA2), an IgD, an IgE, or an IgM isotype Fc.

In some aspects, the antigen binding protein is linked to a scaffold via a linker, optionally wherein the linker is a peptide linker, optionally wherein the peptide linker is a hinge region of a human antibody.

In some aspects, the TCR or antigen-binding portion thereof comprises a TCR variable region. In some aspects, the TCR or antigen-binding portion thereof comprises one or more TCR complementarity determining regions (CDRs). In some aspects, the TCR comprises an alpha chain and a beta chain. In some aspects, the TCR comprises a gamma chain and a delta chain. In some aspects, the TCR comprises a single chain TCR (scTCR). In some aspects, the TCR comprises recombinant TCR sequences. In some aspects, the TCR comprises human TCR sequences, optionally wherein the human TCR sequences are fully-human TCR sequences. In some aspects, the TCR comprises a modified TCRα constant (TRAC) region, a modified TCRP constant (TRBC) region, or a modified TRAC region and a modified TRBC region.

In some aspects, the antigen binding protein comprises a modification that extends half-life.

In some aspects, the antigen binding protein is a portion of a chimeric antigen receptor (CAR) comprising: an extracellular portion comprising the antigen binding protein; and an intracellular signaling domain. In some aspects, the intracellular signaling domain comprises an ITAM. In some aspects, the intracellular signaling domain comprises a signaling domain of a zeta chain of a CD3-zeta (CD3) chain.

In some aspects, the antigen binding protein further comprises a transmembrane domain linking the extracellular domain and the intracellular signaling domain. In some aspects, the transmembrane domain comprises a transmembrane portion of CD28.

In some aspects, the antigen binding protein further comprises an intracellular signaling domain of a T cell costimulatory molecule. In some aspects, the T cell costimulatory molecule is CD28, 4-1BB, OX-40, ICOS, or any combination thereof.

Also provided for herein is a medicament comprising any one of the ABPs described herein.

Also provided for herein is an ABP for use in treatment of cancer, optionally wherein the cancer expresses or is predicted to express the HLA-PEPTIDE antigen a medicament, comprising any one of the ABPs described herein. In some aspects, the cancer is selected from a solid tumor and a hematological tumor.

Also provided for herein is an antigen binding protein (ABP) that competes for binding with any one of the ABPs described herein.

Also provided for herein is an antigen binding protein (ABP) that binds the same HLA-PEPTIDE antigen epitope bound by any one of the ABPs described herein.

Also provided for herein is an engineered cell expressing a receptor comprising the antigen binding protein of any one of the ABPs described herein. In some aspects, the engineered cell is a T cell. In some aspects, the T cell is selected from the group consisting of: a naive T (TN) cell, an effector T cell (TEFF), a memory T cell, a stem cell memory T cell (TSCM), a central memory T cell (TCM), an effector memory T cell (TEM), a terminally differentiated effector memory T cell, a tumor-infiltrating lymphocyte (TIL), an immature T cell, a mature T cell, a helper T cell, a cytotoxic T cell, a mucosa-associated invariant T (MALT) cell, a regulatory T cell (Treg), a TH1 cell, a TH2 cell, a TH3 cell, a TH17 cell, a TH9 cell, a TH22 cell, a follicular helper T cell, an natural killer T cell (NKT), an alpha-beta T cell, and a gamma-delta T cell. In some aspects, the T cell is a cytotoxic T cell (CTL). In some aspects, the engineered cell is a human cell or a human-derived cell. In some aspects, the engineered cell is an autologous cell of a subject. In some aspects, the subject is known or suspected to have cancer. In some aspects, the autologous cell is an isolated cell from a subject. In some aspects, the isolated cell is an an ex vivo cultured cell, optionally wherein the vivo cultured cell is a stimulated cell. In some aspects, the autologous cell is an in vivo-engineered cell. In some aspects, the antigen binding protein is expressed from a heterologous promoter. In some aspects, the ABP comprises a T cell receptor (TCR) or an antigen-binding portion thereof, and wherein a polynucleotide encoding the T cell receptor (TCR) or antigen-binding portion thereof is inserted in an endogenous TCR locus. In some aspects, the engineered cell does not express an endogenous ABP.

Also provided for herein is an isolated polynucleotide or set of polynucleotides encoding the ABP of any one of the ABPs described herein. Also provided for herein is a vector or set of vectors comprising any one of the polynucleotides or set of polynucleotides described herein. Also provided for herein is a virus comprising any one of the polynucleotides or set of polynucleotides described herein. In some aspects, the virus is a filamentous phage.

Also provided for herein is a yeast cell comprising any one of the polynucleotides or set of polynucleotides described herein.

Also provided for herein is a host cell comprising any one of the polynucleotides or set of polynucleotides described herein, optionally wherein the host cell is CHO or HEK293, or optionally wherein the host cell is a T cell.

Also provided for herein is a method of producing an antigen binding protein comprising expressing the antigen binding protein with any one of the host cells described herein and isolating the expressed antigen binding protein.

Also provided for herein is a pharmaceutical composition comprising any one of the antigen binding proteins described herein and a pharmaceutically acceptable excipient.

Also provided for herein is a method of treating cancer in a subject, comprising administering to the subject any one of the antigen binding proteins described herein, any one of the engineered cells described herein, or any one of the pharmaceutical compositions described herein, optionally wherein the cancer is selected from a solid tumor and a hematological tumor.

Also provided for herein is a method of stimulating an immune response in a subject, comprising administering to the subject any one of the antigen binding proteins described herein, any one of the engineered cells described herein, or any one of the pharmaceutical compositions described herein, optionally wherein the cancer is selected from a solid tumor and a hematological tumor.

Also provided for herein is a method of killing a target cell in a subject, comprising administering to the subject any one of the antigen binding proteins described herein, any one of the engineered cells described herein, or any one of the pharmaceutical compositions described herein, optionally wherein the cancer is selected from a solid tumor and a hematological tumor.

In some aspects, the subject is a human subject.

In some aspects, the cancer expresses or is predicted to express an HLA-PEPTIDE antigen or HLA Class I molecule as described in any one of In some aspects, the cancer expresses or is predicted to express an HLA-PEPTIDE antigen comprising an HLA-restricted peptide complexed with an HLA Class I molecule, wherein the HLA-restricted peptide is located in the peptide binding groove of an α1/α2 heterodimer portion of the HLA Class I molecule, wherein the HLA Class I molecule and the HLA-restricted peptide are each selected from an HLA-PEPTIDE antigen as described in any one of SEQ ID NOs:10,755 to 29,364, and wherein the ABP binds to the HLA-PEPTIDE antigen. In some aspects, the HLA-PEPTIDE antigen is selected from the group consisting of: a RAS_G12C MHC Class I antigen comprising HLA-A*02:01 and the restricted peptide KLVVVGACGV; a RAS_G12D MHC Class I antigen comprising HLA-A*11:01 and the restricted peptide VVVGADGVGK; a RAS_G12V MHC Class I antigen comprising HLA-A*11:01 and the restricted peptide VVVGAVGVGK; a CTNNB1_S45P MHC Class I antigen comprising HLA-A*03:01 and the restricted peptide TTAPPLSGK; a RAS_G12D MHC Class I antigen comprising HLA-A*11:01 and the restricted peptide VVGADGVGK; a RAS_G12V MHC Class I antigen comprising HLA-A*11:01 and the restricted peptide VVGAVGVGK; a RAS_G12V MHC Class I antigen comprising HLA-C*01:02 and the restricted peptide AVGVGKSAL; a RAS_G12V MHC Class I antigen comprising HLA-A*03:01 and the restricted peptide VVVGAVGVGK; a TP53_K132N MHC Class I antigen comprising HLA-A*24:02 and the restricted peptide TYSPALNNMF; a CTNNB1_S37Y MHC Class I antigen comprising HLA-A*02:01 and the restricted peptide YLDSGIHYGA; a RAS_G12C MHC Class I antigen comprising HLA-A*03:01 and the restricted peptide VVVGACGVGK; a RAS_G12C MHC Class I antigen comprising HLA-A*11:01 and the restricted peptide VVVGACGVGK; a RAS_G12D MHC Class I antigen comprising HLA-A*03:01 and the restricted peptide VVVGADGVGK; a RAS_Q61H MHC Class I antigen comprising HLA-A*01:01 and the restricted peptide ILDTAGHEEY; and a TP53_R213L MHC Class I antigen comprising A*02:01 and the restricted peptide YLDDRNTFL.

In some aspects, the restricted peptide comprises a RAS_G12A mutation, and wherein the HLA Class I molecule is HLA-A*02:01; the restricted peptide comprises a RAS_G12A mutation, and wherein the HLA Class I molecule is HLA-A*02:06; the restricted peptide comprises a RAS_G12A mutation, and wherein the HLA Class I molecule is HLA-B*27:05; the restricted peptide comprises a RAS_G12A mutation, and wherein the HLA Class I molecule is HLA-B*35:01; the restricted peptide comprises a RAS_G12A mutation, and wherein the HLA Class I molecule is HLA-B*41:02; the restricted peptide comprises a RAS_G12A mutation, and wherein the HLA Class I molecule is HLA-B*48:01; the restricted peptide comprises a RAS_G12A mutation, and wherein the HLA Class I molecule is HLA-C*08:03; the restricted peptide comprises a RAS_G12C mutation, and wherein the HLA Class I molecule is HLA-A*02:01; the restricted peptide comprises a RAS_G12C mutation, and wherein the HLA Class I molecule is HLA-A*02:01; the restricted peptide comprises a RAS_G12C mutation, and wherein the HLA Class I molecule is HLA-A*03:01; the restricted peptide comprises a RAS_G12C mutation, and wherein the HLA Class I molecule is HLA-A*03:02; the restricted peptide comprises a RAS_G12C mutation, and wherein the HLA Class I molecule is HLA-A*68:01; the restricted peptide comprises a RAS_G12C mutation, and wherein the HLA Class I molecule is HLA-B*27:05; the restricted peptide comprises a RAS_G12D mutation, and wherein the HLA Class I molecule is HLA-A*02:01; the restricted peptide comprises a RAS_G12D mutation, and wherein the HLA Class I molecule is HLA-A*02:05; the restricted peptide comprises a RAS_G12D mutation, and wherein the HLA Class I molecule is HLA-A*03:01; the restricted peptide comprises a RAS_G12D mutation, and wherein the HLA Class I molecule is HLA-A*11:01; the restricted peptide comprises a RAS_G12D mutation, and wherein the HLA Class I molecule is HLA-A*11:01; the restricted peptide comprises a RAS_G12D mutation, and wherein the HLA Class I molecule is HLA-A*26:01; the restricted peptide comprises a RAS_G12D mutation, and wherein the HLA Class I molecule is HLA-A*31:01; the restricted peptide comprises a RAS_G12D mutation, and wherein the HLA Class I molecule is HLA-A*68:01; the restricted peptide comprises a RAS_G12D mutation, and wherein the HLA Class I molecule is HLA-B*07:02; the restricted peptide comprises a RAS_G12D mutation, and wherein the HLA Class I molecule is HLA-B*08:01; the restricted peptide comprises a RAS_G12D mutation, and wherein the HLA Class I molecule is HLA-B*13:02; the restricted peptide comprises a RAS_G12D mutation, and wherein the HLA Class I molecule is HLA-B*15:01; the restricted peptide comprises a RAS_G12D mutation, and wherein the HLA Class I molecule is HLA-B*27:05; the restricted peptide comprises a RAS_G12D mutation, and wherein the HLA Class I molecule is HLA-B*35:01; the restricted peptide comprises a RAS_G12D mutation, and wherein the HLA Class I molecule is HLA-B*37:01; the restricted peptide comprises a RAS_G12D mutation, and wherein the HLA Class I molecule is HLA-B*38:01; the restricted peptide comprises a RAS_G12D mutation, and wherein the HLA Class I molecule is HLA-B*40:01; the restricted peptide comprises a RAS_G12D mutation, and wherein the HLA Class I molecule is HLA-B*40:02; the restricted peptide comprises a RAS_G12D mutation, and wherein the HLA Class I molecule is HLA-B*44:02; the restricted peptide comprises a RAS_G12D mutation, and wherein the HLA Class I molecule is HLA-B*44:03; the restricted peptide comprises a RAS_G12D mutation, and wherein the HLA Class I molecule is HLA-B*48:01; the restricted peptide comprises a RAS_G12D mutation, and wherein the HLA Class I molecule is HLA-B*50:01; the restricted peptide comprises a RAS_G12D mutation, and wherein the HLA Class I molecule is HLA-B*57:01; the restricted peptide comprises a RAS_G12D mutation, and wherein the HLA Class I molecule is HLA-C*01:02; the restricted peptide comprises a RAS_G12D mutation, and wherein the HLA Class I molecule is HLA-C*02:02; the restricted peptide comprises a RAS_G12D mutation, and wherein the HLA Class I molecule is HLA-C*03:03; the restricted peptide comprises a RAS_G12D mutation, and wherein the HLA Class I molecule is HLA-C*03:04; the restricted peptide comprises a RAS_G12D mutation, and wherein the HLA Class I molecule is HLA-C*04:01; the restricted peptide comprises a RAS_G12D mutation, and wherein the HLA Class I molecule is HLA-C*05:01; the restricted peptide comprises a RAS_G12D mutation, and wherein the HLA Class I molecule is HLA-C*07:04; the restricted peptide comprises a RAS_G12D mutation, and wherein the HLA Class I molecule is HLA-C*08:02; the restricted peptide comprises a RAS_G12D mutation, and wherein the HLA Class I molecule is HLA-C*08:03; the restricted peptide comprises a RAS_G12D mutation, and wherein the HLA Class I molecule is HLA-C*16:01; the restricted peptide comprises a RAS_G12D mutation, and wherein the HLA Class I molecule is HLA-C*17:01; the restricted peptide comprises a RAS_G12D mutation, and wherein the HLA Class I molecule is HLA-B*41:02; the restricted peptide comprises a RAS_G12D mutation, and wherein the HLA Class I molecule is HLA-C*07:04; the restricted peptide comprises a RAS_G12V mutation, and wherein the HLA Class I molecule is HLA-A*02:01; the restricted peptide comprises a RAS_G12V mutation, and wherein the HLA Class I molecule is HLA-A*02:05; the restricted peptide comprises a RAS_G12V mutation, and wherein the HLA Class I molecule is HLA-A*02:06; the restricted peptide comprises a RAS_G12V mutation, and wherein the HLA Class I molecule is HLA-A*03:01; the restricted peptide comprises a RAS_G12V mutation, and wherein the HLA Class I molecule is HLA-A*03:01; the restricted peptide comprises a RAS_G12V mutation, and wherein the HLA Class I molecule is HLA-A*11:01; the restricted peptide comprises a RAS_G12V mutation, and wherein the HLA Class I molecule is HLA-A*11:01; the restricted peptide comprises a RAS_G12V mutation, and wherein the HLA Class I molecule is HLA-A*25:01; the restricted peptide comprises a RAS_G12V mutation, and wherein the HLA Class I molecule is HLA-A*26:01; the restricted peptide comprises a RAS_G12V mutation, and wherein the HLA Class I molecule is HLA-A*30:01; the restricted peptide comprises a RAS_G12V mutation, and wherein the HLA Class I molecule is HLA-A*31:01; the restricted peptide comprises a RAS_G12V mutation, and wherein the HLA Class I molecule is HLA-A*31:01; the restricted peptide comprises a RAS_G12V mutation, and wherein the HLA Class I molecule is HLA-A*32:01; the restricted peptide comprises a RAS_G12V mutation, and wherein the HLA Class I molecule is HLA-A*68:02; the restricted peptide comprises a RAS_G12V mutation, and wherein the HLA Class I molecule is HLA-B*07:02; the restricted peptide comprises a RAS_G12V mutation, and wherein the HLA Class I molecule is HLA-B*08:01; the restricted peptide comprises a RAS_G12V mutation, and wherein the HLA Class I molecule is HLA-B*13:02; the restricted peptide comprises a RAS_G12V mutation, and wherein the HLA Class I molecule is HLA-B*14:02; the restricted peptide comprises a RAS_G12V mutation, and wherein the HLA Class I molecule is HLA-B*15:01; the restricted peptide comprises a RAS_G12V mutation, and wherein the HLA Class I molecule is HLA-B*27:05; the restricted peptide comprises a RAS_G12V mutation, and wherein the HLA Class I molecule is HLA-B*39:01; the restricted peptide comprises a RAS_G12V mutation, and wherein the HLA Class I molecule is HLA-B*40:01; the restricted peptide comprises a RAS_G12V mutation, and wherein the HLA Class I molecule is HLA-B*40:02; the restricted peptide comprises a RAS_G12V mutation, and wherein the HLA Class I molecule is HLA-B*41:02; the restricted peptide comprises a RAS_G12V mutation, and wherein the HLA Class I molecule is HLA-B*44:05; the restricted peptide comprises a RAS_G12V mutation, and wherein the HLA Class I molecule is HLA-B*50:01; the restricted peptide comprises a RAS_G12V mutation, and wherein the HLA Class I molecule is HLA-B*51:01; the restricted peptide comprises a RAS_G12V mutation, and wherein the HLA Class I molecule is HLA-C*01:02; the restricted peptide comprises a RAS_G12V mutation, and wherein the HLA Class I molecule is HLA-C*01:02; the restricted peptide comprises a RAS_G12V mutation, and wherein the HLA Class I molecule is HLA-C*03:03; the restricted peptide comprises a RAS_G12V mutation, and wherein the HLA Class I molecule is HLA-C*03:04; the restricted peptide comprises a RAS_G12V mutation, and wherein the HLA Class I molecule is HLA-C*08:02; the restricted peptide comprises a RAS_G12V mutation, and wherein the HLA Class I molecule is HLA-C*14:02; the restricted peptide comprises a RAS_G12V mutation, and wherein the HLA Class I molecule is HLA-C*17:01; the restricted peptide comprises a KRAS_G13D mutation, and wherein the HLA Class I molecule is HLA-A*02:01; the restricted peptide comprises a KRAS_G13D mutation, and wherein the HLA Class I molecule is HLA-B*07:02; the restricted peptide comprises a KRAS_G13D mutation, and wherein the HLA Class I molecule is HLA-B*08:01; the restricted peptide comprises a KRAS_G13D mutation, and wherein the HLA Class I molecule is HLA-B*35:01; the restricted peptide comprises a KRAS_G13D mutation, and wherein the HLA Class I molecule is HLA-B*35:03; the restricted peptide comprises a KRAS_G13D mutation, and wherein the HLA Class I molecule is HLA-B*35:08; the restricted peptide comprises a KRAS_G13D mutation, and wherein the HLA Class I molecule is HLA-B*38:01; the restricted peptide comprises a KRAS_G13D mutation, and wherein the HLA Class I molecule is HLA-C*04:01; the restricted peptide comprises a KRAS_Q61H mutation, and wherein the HLA Class I molecule is HLA-A*01:01; the restricted peptide comprises a KRAS_Q61H mutation, and wherein the HLA Class I molecule is HLA-A*02:01; the restricted peptide comprises a KRAS_Q61H mutation, and wherein the HLA Class I molecule is HLA-A*23:01; the restricted peptide comprises a KRAS_Q61H mutation, and wherein the HLA Class I molecule is HLA-A*29:01; the restricted peptide comprises a KRAS_Q61H mutation, and wherein the HLA Class I molecule is HLA-A*30:02; the restricted peptide comprises a KRAS_Q61H mutation, and wherein the HLA Class I molecule is HLA-A*33:01; the restricted peptide comprises a KRAS_Q61H mutation, and wherein the HLA Class I molecule is HLA-A*68:01; the restricted peptide comprises a KRAS_Q61H mutation, and wherein the HLA Class I molecule is HLA-B*07:02; the restricted peptide comprises a KRAS_Q61H mutation, and wherein the HLA Class I molecule is HLA-B*08:01; the restricted peptide comprises a KRAS_Q61H mutation, and wherein the HLA Class I molecule is HLA-B*18:01; the restricted peptide comprises a KRAS_Q61H mutation, and wherein the HLA Class I molecule is HLA-B*35:01; the restricted peptide comprises a KRAS_Q61H mutation, and wherein the HLA Class I molecule is HLA-B*38:01; the restricted peptide comprises a KRAS_Q61H mutation, and wherein the HLA Class I molecule is HLA-B*40:01; the restricted peptide comprises a KRAS_Q61H mutation, and wherein the HLA Class I molecule is HLA-B*44:02; the restricted peptide comprises a KRAS_Q61H mutation, and wherein the HLA Class I molecule is HLA-C*03:04; the restricted peptide comprises a KRAS_Q61H mutation, and wherein the HLA Class I molecule is HLA-C*05:01; or the restricted peptide comprises a KRAS_Q61H mutation, and wherein the HLA Class I molecule is HLA-C*08:02.

In some aspects, the restricted peptide comprises a KRAS_G13D mutation, and wherein the HLA Class I molecule is C*08:02 or A*11:01; the restricted peptide comprises a KRAS_Q61K mutation, and wherein the HLA Class I molecule is A*01:01; the restricted peptide comprises a NRAS_Q61K mutation, and wherein the HLA Class I molecule is A*01:01; the restricted peptide comprises a TP53_R249M mutation, and wherein the HLA Class I molecule is B*35:12, B*35:03, or B*35:01; the restricted peptide comprises a CTNNB1_S45P mutation, and wherein the HLA Class I molecule is A*03:01, A*11:01, A*68:01, or A*03:02; the restricted peptide comprises a CTNNB1_S45F mutation, and wherein the HLA Class I molecule is A*03:01, A*11:01, or A*68:01; the restricted peptide comprises a ERBB2_Y772_A775dup mutation, and wherein the HLA Class I molecule is B*18:01; the restricted peptide comprises a KRAS_G12D mutation, and wherein the HLA Class I molecule is A*11:01, A*03:01, or C*08:02; the restricted peptide comprises a NRAS_G12D mutation, and wherein the HLA Class I molecule is A*11:01, A*03:01, or C*08:02; the restricted peptide comprises a KRAS_Q61D mutation, and wherein the HLA Class I molecule is A*01:01; the restricted peptide comprises a NRAS_Q61D mutation, and wherein the HLA Class I molecule is A*01:01; the restricted peptide comprises a CTNNB1_T41A mutation, and wherein the HLA Class I molecule is A*03:01, A*0302, A*11:01, B*15:10, C*03:03, or C*03:04; the restricted peptide comprises a TP53_K132N mutation, and wherein the HLA Class I molecule is A*24:02 or A*23:01; the restricted peptide comprises a KRAS_G12A mutation, and wherein the HLA Class I molecule is A*03:01 or A*11:01; the restricted peptide comprises a KRAS_Q61L mutation, and wherein the HLA Class I molecule is A*01:01; the restricted peptide comprises a NRAS_Q61L mutation, and wherein the HLA Class I molecule is A*01:01; the restricted peptide comprises a TP53_R213L mutation, and wherein the HLA Class I molecule is A*02:07, C*08:02, or A*02:01; the restricted peptide comprises a BRAF_G466V mutation, and wherein the HLA Class I molecule is B*15:01, or B*15:03; the restricted peptide comprises a KRAS_G12V mutation, and wherein the HLA Class I molecule is A*03:01, A*03:02, A*11:01, or C*01:02; the restricted peptide comprises a KRAS_Q61H mutation, and wherein the HLA Class I molecule is A*01:01; the restricted peptide comprises a NRAS_Q61H mutation, and wherein the HLA Class I molecule is A*01:01; the restricted peptide comprises a CTNNB1_S37F mutation, and wherein the HLA Class I molecule is A*01:01, A*23:01, A*24:02, B*15:10, B*39:06, C*05:01, C*14:02, or C*14:03; the restricted peptide comprises a TP53_S127Y mutation, and wherein the HLA Class I molecule is A*11:01 or A*03:01; the restricted peptide comprises a TP53_K132E mutation, and wherein the HLA Class I molecule is A*24:02, C*14:03, or A*23:01; the restricted peptide comprises a KRAS_G12C mutation, and wherein the HLA Class I molecule is A*02:01, A*11:01, or A*03:01; the restricted peptide comprises a NRAS_G12C mutation, and wherein the HLA Class I molecule is A*02:01, A*11:01, or A*03:01; the restricted peptide comprises a EGFR_L858D mutation, and wherein the HLA Class I molecule is A*11:01, or A*03:01; the restricted peptide comprises a TP53_Y220C mutation, and wherein the HLA Class I molecule is A*02:01; or

the restricted peptide comprises a TP53_R175H mutation, and wherein the HLA Class I molecule is A*02:01.

In some aspects, the HLA-PEPTIDE antigen is selected from: a CTNNB1_S45P MHC Class I antigen comprising A*11:01 and the restricted peptide TTAPPLSGK; a CTNNB1_T41A MHC Class I antigen comprising A*11:01 and the restricted peptide ATAPSLSGK; a RAS_G12D MHC Class I antigen comprising A*11:01 and the restricted peptide VVVGADGVGK; a RAS_G12V MHC Class I antigen comprising A*03:01 and the restricted peptide VVGAVGVGK; a RAS_G12V MHC Class I antigen comprising A*03:01 and the restricted peptide VVVGAVGVGK; a RAS_G12V MHC Class I antigen comprising A*11:01 and the restricted peptide VVGAVGVGK; a RAS_G12V MHC Class I antigen comprising A*11:01 and the restricted peptide VVVGAVGVGK; a KRAS_Q61R MHC Class I antigen comprising A*01:01 and the restricted peptide ILDTAGREEY; and a TP53_R213L MHC Class I antigen comprising A*02:01 and the restricted peptide YLDDRNTFL.

In some aspects, the HLA-PEPTIDE antigen comprises an HLA-restricted peptide which is a peptide fragment of RAS comprising a RAS G12 mutation. In some aspects, the G12 mutation is a G12C, a G12D, a G12V, or a G12A mutation. In some aspects, the HLA-PEPTIDE antigen comprises an HLA Class I molecule selected from HLA-A*02:01, HLA-A*11:01, HLA-A*31:01, HLA-C*01:02, and HLA-A*03:01. In some aspects, the RAS G12 mutation is any one or more of a KRAS, NRAS, and HRAS mutation. In some aspects, the HLA-PEPTIDE antigen is selected from: a RAS_G12C MHC Class I antigen comprising HLA-A*02:01 and the restricted peptide KLVVVGACGV; a RAS_G12C MHC Class I antigen comprising HLA-A*03:01 and the restricted peptide VVVGACGVGK; a RAS_G12C MHC Class I antigen comprising HLA-A*11:01 and the restricted peptide VVVGACGVGK; a RAS_G12D MHC Class I antigen comprising HLA-A*11:01 and the restricted peptide VVVGADGVGK; a RAS_G12D MHC Class I antigen comprising HLA-A*11:01 and the restricted peptide VVGADGVGK; a RAS_G12D MHC Class I antigen comprising HLA-A*03:01 and the restricted peptide VVVGADGVGK; a RAS_G12V MHC Class I antigen comprising HLA-A*11:01 and the restricted peptide VVVGAVGVGK; a RAS_G12V MHC Class I antigen comprising HLA-A*31:01 and the restricted peptide VVVGAVGVGK; a RAS_G12V MHC Class I antigen comprising HLA-A*11:01 and the restricted peptide VVGAVGVGK; a RAS_G12V MHC Class I antigen comprising HLA-C*01:02 and the restricted peptide AVGVGKSAL; and a RAS_G12V MHC Class I antigen comprising HLA-A*03:01 and the restricted peptide VVVGAVGVGK. In some aspects, the HLA-PEPTIDE antigen is selected from: a RAS_G12C MHC Class I antigen comprising HLA-A*02:01 and the restricted peptide KLVVVGACGV; a RAS_G12D MHC Class I antigen comprising HLA-A*11:01 and the restricted peptide VVVGADGVGK; a RAS_G12D MHC Class I antigen comprising HLA-A*11:01 and the restricted peptide VVGADGVGK; a RAS_G12V MHC Class I antigen comprising HLA-A*11:01 and the restricted peptide VVVGAVGVGK; a RAS_G12V MHC Class I antigen comprising HLA-A*31:01 and the restricted peptide VVVGAVGVGK; a RAS_G12V MHC Class I antigen comprising HLA-A*11:01 and the restricted peptide VVGAVGVGK; a RAS_G12V MHC Class I antigen comprising HLA-C*01:02 and the restricted peptide AVGVGKSAL; and a RAS_G12V MHC Class I antigen comprising HLA-A*03:01 and the restricted peptide VVVGAVGVGK. In some aspects, the HLA-PEPTIDE antigen is selected from: a RAS_G12C MHC Class I antigen comprising HLA-A*02:01 and the restricted peptide KLVVVGACGV; a RAS_G12D MHC Class I antigen comprising HLA-A*11:01 and the restricted peptide VVVGADGVGK; or a RAS_G12V MHC Class I antigen comprising HLA-A*11:01 and the restricted peptide VVVGAVGVGK. In some aspects, the antigen binding protein binds to a RAS_G12C MHC Class I antigen comprising HLA-A*02:01 and the restricted peptide KLVVVGACGV, and wherein the ABP binds to the RAS_G12C MHC Class I antigen at a higher affinity than an HLA-PEPTIDE antigen comprising a different RAS G12 mutation. In some aspects, the ABP binds to the RAS_G12C MHC Class I antigen at a higher affinity than an HLA-PEPTIDE antigen comprising the restricted peptide KLVVVGAVGV and an HLA-A2 molecule. In some aspects, the ABP does not bind to an HLA-PEPTIDE antigen comprising the restricted peptide KLVVVGAVGV and an HLA-A2 molecule.

In some aspects, the HLA-PEPTIDE antigen comprises an HLA-restricted peptide which is a peptide fragment of RAS comprising a RAS Q61 mutation. In some aspects, the Q61 mutation is a Q61H, a Q61K, a Q61R, or a Q61L mutation. In some aspects, the HLA-PEPTIDE antigen is a RAS_Q61H MHC Class I antigen comprising HLA-A*01:01 and the restricted peptide ILDTAGHEEY.

In some aspects, the HLA-PEPTIDE antigen comprises an HLA-restricted peptide which is a peptide fragment of TP53 comprising a TP53 mutation. In some aspects, the TP53 mutation comprises a R213L, S127Y, Y220C, R175H, or R249M mutation. In some aspects, the HLA-PEPTIDE antigen is a TP53 R213L MHC Class I antigen comprising A*02:01 and the restricted peptide YLDDRNTFL.

In some aspects, the method comprises, prior to the administering, determining or having determined the presence of any one or more of the HLA-PEPTIDE antigen, the peptide of the HLA-PEPTIDE antigen, the somatic mutation associated with the HLA-PEPTIDE antigen, and the HLA molecule of the HLA-PEPTIDE antigen in a biological sample obtained from the subject.

In some aspects, the biological sample is a blood sample or a tumor sample. In some aspects, the blood sample is a plasma or serum sample.

In some aspects, the determining comprises RNASeq, microarray, PCR, Nanostring, in situ hybridization (ISH), Mass spectrometry, sequencing, or immunohistochemistry (IHC).

In some aspects of the method, the method comprises, after having determined the presence of the HLA-PEPTIDE antigen, peptide, or HLA in the biological sample obtained from the subject, administering to the subject an ABP that selectively binds to the HLA-PEPTIDE antigen.

Also provided herein is a kit comprising the antigen binding protein disclosed herein or a pharmaceutical composition disclosed herein and instructions for use.

Also provided herein is a system, comprising: an isolated HLA-PEPTIDE antigen comprising an HLA-restricted peptide complexed with an HLA Class I molecule, wherein the HLA-restricted peptide is located in the peptide binding groove of an α1/α2 heterodimer portion of the HLA Class I molecule, and wherein the HLA-PEPTIDE antigen is selected from an HLA-PEPTIDE antigen described in any one of SEQ ID NOs:10,755 to 29,364; and a phage display library.

In some aspects, the HLA-PEPTIDE antigen is attached to a solid support. In some aspects, the solid support comprises a bead, well, membrane, tube, column, plate, sepharose, magnetic bead, cell, or chip. In some aspects, the HLA-PEPTIDE antigen comprises a first member of an affinity binding pair and the solid support comprises a second member of the affinity binding pair. In some aspects, the first member is streptavidin and the second member is biotin.

In some aspects, the phage display library is a human library. In some aspects, the phage display library is a humanized library.

In some aspects, the system further comprises a negative control HLA-PEPTIDE antigen comprising an HLA-restricted peptide complexed with an HLA Class I molecule, wherein the HLA-restricted peptide is located in the peptide binding groove of an α1/α2 heterodimer portion of the HLA Class I molecule, and wherein the negative control HLA-PEPTIDE antigen comprises a different restricted peptide, a different HLA Class I molecule, or a different restricted peptide and a different HLA Class I molecule. In some aspects, the negative control HLA-PEPTIDE antigen comprises a different restricted peptide but the same HLA Class I molecule as the HLA-PEPTIDE antigen.

In some aspects, the system comprises a reaction mixture, the reaction mixture comprising the HLA-PEPTIDE antigen and a plurality of phages from the phage display library.

Also provided herein is use of a system disclosed herein for identifying an antigen binding protein that selectively binds the isolated HLA-PEPTIDE antigen.

Also provided herein is a composition comprising an HLA-PEPTIDE antigen as described by any one of SEQ ID NOs:10,755 to 29,364, wherein the HLA-PEPTIDE antigen is covalently linked to an affinity tag. In some aspects, the affinity tag is a biotin tag.

Also provided herein is a composition comprising an HLA-PEPTIDE antigen as described by any one of SEQ ID NOs:10,755 to 29,364 complexed with a detectable label. In some aspects, the detectable label comprises a β₂-microglobulin binding molecule. In some aspects, the β₂-microglobulin binding molecule is a labeled antibody. In some aspects, the labeled antibody is a fluorochrome-labeled antibody.

Also provided herein is a composition comprising an HLA-PEPTIDE antigen as described by any one of SEQ ID NOs:10,755 to 29,364 attached to a solid support. In some aspects, the solid support comprises a bead, well, membrane, tube, column, plate, sepharose, magnetic bead, cell, or chip. In some aspects, the HLA-PEPTIDE antigen comprises a first member of an affinity binding pair and the solid support comprises a second member of the affinity binding pair. In some aspects, the first member is streptavidin and the second member is biotin.

Also provided herein is a host cell comprising a heterologous HLA-PEPTIDE antigen as described by any one of SEQ ID NOs:10,755-29,364. Also provided herein is a host cell which expresses an HLA subtype as defined by any one of the HLA-PEPTIDE antigens described in SEQ ID NOs:10,755-29,364. Also provided herein is a host cell comprising a polynucleotide encoding an HLA-restricted peptide as defined by any one of the HLA-PEPTIDE antigens in SEQ ID NOs:10,755-29,364.

In some aspects, the host cell does not comprise endogenous MHC. In some aspects, the host cell comprises an exogenous HLA. In some aspects, the host cell is a K562 or A375 cell. In some aspects, the host cell is a cultured cell from a tumor cell line. In some aspects, the tumor cell line expresses an HLA subtype as defined by the same HLA-PEPTIDE antigen that describes the HLA-restricted peptide. In some aspects, the tumor cell line is selected from the group consisting of HCC-1599, NCI-H510A, A375, LN229, NCI-H358, ZR-75-1, MS751, OE19, MOR, BV173, MCF-7, NCI-H82, Colo829, SK-MEL-28, KYSE270, 59M, and NCI-H146.

Also provided herein is a cell culture system comprising a host cell disclosed herein, and a cell culture medium. In some aspects, the host cell expresses an HLA subtype as defined by any one of the HLA-PEPTIDE antigens in SEQ ID NOs:10,755-21,015 and SEQ ID NOs: 21,016-29,364, and wherein the cell culture medium comprises a restricted peptide as defined by the same HLA-PEPTIDE antigen as the HLA subtype. In some aspects, the host cell is a K562 cell which comprises an exogenous HLA, wherein the exogenous HLA is an HLA subtype as defined by any one of the HLA-PEPTIDE antigens in SEQ ID NOs:10,755-29,364, and the cell culture medium comprises a restricted peptide as defined by the same HLA-PEPTIDE antigen defining the HLA subtype.

Also provided herein is a method of identifying an antigen binding protein disclosed herein, comprising providing at least one HLA-PEPTIDE antigen described in SEQ ID NOs:10,755-29,364; and binding the at least one target with the antigen binding protein, thereby identifying the antigen binding protein.

In some aspects, the antigen binding protein is present in a phage display library comprising a plurality of distinct antigen binding proteins. In some aspects, the phage display library is substantially free of antigen binding proteins that non-specifically bind the HLA of the HLA-PEPTIDE antigen.

In some aspects, the binding step is performed more than once, optionally at least three times.

In some aspects, the method further comprises contacting the antigen binding protein with one or more peptide-HLA complexes that are distinct from the HLA-PEPTIDE antigen to determine if the antigen binding protein selectively binds the HLA-PEPTIDE antigen, optionally wherein selectivity is determined by measuring binding affinity of the antigen binding protein to soluble target HLA-PEPTIDE complexes versus soluble HLA-PEPTIDE complexes that are distinct from target complexes, optionally wherein selectivity is determined by measuring binding affinity of the antigen binding protein to target HLA-PEPTIDE complexes expressed on the surface of one or more cells versus HLA-PEPTIDE complexes that are distinct from target complexes expressed on the surface of one or more cells.

Also provided herein is a method of identifying an antigen binding protein disclosed herein, comprising obtaining at least one HLA-PEPTIDE antigen described in SEQ ID NOs:10,755-29,364; administering the HLA-PEPTIDE antigen to a subject, optionally in combination with an adjuvant; and isolating the antigen binding protein from the subject.

In some aspects, isolating the antigen binding protein comprises screening the serum of the subject to identify the antigen binding protein.

In some aspects, the method further comprises contacting the antigen binding protein with one or more peptide-HLA complexes that are distinct from the HLA-PEPTIDE antigen to determine if the antigen binding protein selectively binds to the HLA-PEPTIDE antigen, optionally wherein selectivity is determined by measuring binding affinity of the antigen binding protein to the HLA-PEPTIDE antigen versus soluble HLA-PEPTIDE complexes that are distinct from the HLA-PEPTIDE antigen, optionally wherein selectivity is determined by measuring binding affinity of the antigen binding protein to the HLA-PEPTIDE antigen expressed on the surface of one or more cells versus HLA-PEPTIDE complexes that are distinct from the HLA-PEPTIDE antigen expressed on the surface of one or more cells.

In some aspects, the subject is a mouse, a rabbit, or a llama.

In some aspects, isolating the antigen binding protein comprises isolating a B cell from the subject that expresses the antigen binding protein and optionally directly cloning sequences encoding the antigen binding protein from the isolated B cell. In some aspects, the method further comprises creating a hybridoma using the B cell. In some aspects, the method further comprises cloning CDRs from the B cell. In some aspects, the method further comprises immortalizing the B cell, optionally via EBV transformation.

In some aspects, the method further comprises creating a library that comprises the antigen binding protein of the B cell, optionally wherein the library is phage display or yeast display.

In some aspects, the method further comprises humanizing the antigen binding protein.

Also provided herein is a method of identifying an antigen binding protein disclosed herein, comprising obtaining a cell comprising the antigen binding protein; contacting the cell with an HLA-multimer comprising at least one HLA-PEPTIDE antigen described in SEQ ID NOs:10,755-29,364; and identifying the antigen binding protein via binding between the HLA-multimer and the antigen binding protein. In some aspects, the method further comprises contacting the cell comprising the antigen binding protein with an HLA-multimer comprising a corresponding wildtype sequence of the at least one HLA-PEPTIDE antigen described in SEQ ID NOs:10,755-29,364, and excluding the antigen binding protein if the antigen binding protein binds the HLA-multimer comprising the corresponding wildtype sequence

Also provided herein is a method of identifying an antigen binding protein disclosed herein, comprising providing at least one HLA-PEPTIDE antigen described in SEQ ID NOs:10,755-29,364; and identifying the antigen binding protein using the target.

Also provided herein is an antigen binding protein (ABP) that specifically binds to an HLA-PEPTIDE antigen comprising an HLA-restricted peptide complexed with an HLA Class I molecule, wherein the HLA-restricted peptide is located in the peptide binding groove of an α1/α2 heterodimer portion of the HLA Class I molecule, wherein the HLA Class I molecule and the HLA-restricted peptide are each selected from an HLA-PEPTIDE antigen as described in any one of SEQ ID NOs:10,755 to 29,364, and wherein the ABP comprises an alpha-CDR3 amino acid sequence and corresponding beta-CDR3 amino acid sequence selected from the group consisting of the sequences shown in Tables 1C. 10, 1C. 2, 1C. 3, and 1D.

In some aspects, the ABP further comprises an alpha variable (“V”) segment, an alpha joining (“J”) segment, a beta variable (“V”) segment, a beta joining (“J”) segment, optionally a beta diversity (“D”) segment, and optionally a beta constant region selected from the group consisting of the regions shown in Tables 1C. 1, 1C. 2, 1C. 3, and 1D corresponding to the alpha-CDR3 amino acid sequence and corresponding beta-CDR3 amino acid sequence. In some aspects, the ABP comprises an alpha variable region and corresponding beta variable region comprising the amino acid sequences selected from the sequences shown in Tables 1A. 1, 1A. 2, 1A. 3, and 1B corresponding to the alpha-CDR3 amino acid sequence and corresponding beta-CDR3 amino acid sequence.

Also provided herein is an antigen binding protein (ABP) that specifically binds to an HLA-PEPTIDE antigen comprising an HLA-restricted RAS peptide complexed with an HLA Class I molecule, wherein the HLA-restricted peptide is located in the peptide binding groove of an α1/α2 heterodimer portion of the HLA Class I molecule, wherein the HLA-restricted RAS peptide comprises at least one alteration that makes HLA-restricted RAS peptide sequence distinct from the corresponding peptide sequence of a wild-type RAS peptide, and wherein the ABP comprises an alpha-CDR3 amino acid sequence and corresponding beta-CDR3 amino acid sequence selected from the group consisting of the sequences shown in Tables 1C. 1, 1C. 2, 1C. 3, and 1D.

In some aspects, the HLA-PEPTIDE antigen is selected from Table 5A. In some aspects, the HLA-PEPTIDE antigen is selected from Table 5B. In some aspects, the HLA-PEPTIDE antigen is selected from Table 6. In some aspects, HLA-PEPTIDE antigen is selected from Table 7.

In some aspects, HLA-restricted peptide comprises a RAS G12 mutation. In some aspects, the G12 mutation is a G12C, a G12D, a G12V, or a G12A mutation In some aspects, the HLA-PEPTIDE antigen comprises an HLA Class I molecule selected from HLA-A*02:01, HLA-A*11:01, HLA-A*31:01, HLA-C*01:02, and HLA-A*03:01. In some aspects, the RAS G12 mutation is any one or more of: a KRAS, NRAS, and HRAS mutation.

In some aspects, the HLA-PEPTIDE antigen is a RAS_G12C MHC Class I antigen comprising HLA-A*02:01 and the restricted peptide KLVVVGACGV. In some aspects, the ABP comprises an alpha-CDR3 amino acid sequence and corresponding beta-CDR3 amino acid sequence selected from the group consisting of the sequences shown in Table 1C. 2. In some aspects, ABP further comprises an alpha variable (“V”) segment, an alpha joining (“J”) segment, a beta variable (“V”) segment, a beta joining (“J”) segment, optionally a beta diversity (“D”) segment, and optionally a beta constant region selected from the group consisting of the regions shown in Table 1C. 2 corresponding to the alpha-CDR3 amino acid sequence and corresponding beta-CDR3 amino acid sequence. In some aspects, the ABP comprises an alpha variable region and corresponding beta variable region comprising the amino acid sequences selected from the sequences shown in Table 1A. 2 corresponding to the alpha-CDR3 amino acid sequence and corresponding beta-CDR3 amino acid sequence.

In some aspects, the HLA-PEPTIDE antigen is a RAS_G12V MHC Class I antigen comprising HLA-A*11:01 and the restricted peptide VVGAVGVGK. In some aspects, the ABP comprises an alpha-CDR3 amino acid sequence and corresponding beta-CDR3 amino acid sequence selected from the group consisting of the sequences shown in Table 1C. 3. In some aspects, the ABP further comprises an alpha variable (“V”) segment, an alpha joining (“J”) segment, a beta variable (“V”) segment, a beta joining (“J”) segment, optionally a beta diversity (“D”) segment, and optionally a beta constant region selected from the group consisting of the regions shown in Table 1C. 3 corresponding to the alpha-CDR3 amino acid sequence and corresponding beta-CDR3 amino acid sequence. In some aspects, the ABP comprises an alpha variable region and corresponding beta variable region comprising the amino acid sequences selected from the sequences shown in Table 1A. 3 corresponding to the alpha-CDR3 amino acid sequence and corresponding beta-CDR3 amino acid sequence.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 shows the general structure of a Human Leukocyte Antigen (HLA) Class I molecule. By User atropos235 on en.wikipedia—Own work, CC BY 2.5, https://conimons.wikimedia.org/w/index.php?curid=1805424

FIG. 2 depicts flow-cytometry analysis of enriched naïve and memory T cells. Shown are cells labeled using a pool of 6 neoantigen-MHC tetramers (“HLA/SNA”) to identify neoantigen specific T cells (left panel, X-axis) and a pool of MHC-tetramers for the corresponding wildtype peptides (“HLA/wild-type”; left panel, Y-axis). Also shown are cells labeled for the memory T cell phenotype marker CD45RO (right panel).

FIG. 3A depicts flow-cytometry analysis of expanded T cells that were previously sorted using a pool of 6 neoantigen-MHC tetramers (“HLA/SNA”). Shown are the expanded cells labeled with each of the 6 neoantigen-MHC tetramers and their corresponding wildtype peptide-MHC tetramer.

FIG. 3B depicts flow-cytometry analysis of expanded T cells that were previously sorted using neoantigen-MHC tetramers (“HLA/SNA”). Shown are the expanded cells labeled with each of the 4 neoantigen-MHC tetramers and their corresponding wildtype peptide-MHC tetramer.

FIG. 4 depicts the correlation between EDGE score and the probability of detection of candidate shared neoantigen peptides by targeted Mass Spectrometry.

FIG. 5A depicts flow cytometry gating strategy for detecting CD8+ T cells.

FIG. 5B depicts flow cytometry results demonstrating that a large proportion of CD8+ T cells exhibit binding to the RAS G12V:HLA*1101 pHLA.

FIG. 6 depicts depicts flow-cytometry analysis of expanded T cells that were previously sorted using a single neoantigen-MHC tetramer for two different donors. Shown are expanded cells labeled with each of 3 neoantigen-MHC tetramers and their corresponding wildtype peptide-MHC tetramer.

FIG. 7 shows titration of DOX administration in regulating expression of a representative neoantigen under a Tet-On system in multiple K562-HLA cell-lines.

FIG. 8 shows a representative KRAS G12V peptide VVGAVGVGK observed by mass-spectrometry in a HLA-A*11:01 expressing K562 cell line. Top panels shows detection was DOX dependent (left column no DOX; right panel DOX added), and bottom panels show detection of the heavy peptide control standard was equivalent.

FIG. 9 depicts expanded naïve CD8 T gated on CD137+ following neoantigen (left panel) and DMSO (right panel) stimulation.

FIG. 10 illustrates a summary of in silico analysis for shared TCR sequences among (i) neoantigen-tetramer labeled cells; (ii) CD137+ neoantigen-stimulated cells; and (iii) CD137+ DMSO-stimulated cells.

FIG. 11A depicts a representative flow cytometry assessment for TCR clone 01CA019_064_F05_0047. Shown are activation markers CD25 (left panels), CD69 (middle panels), and CD137 (right panels) in primary T cells transduced with the indicated TCR and stimulated with a cognate neoantigen (bottom panels) or corresponding wildtype peptide (top panels.

FIG. 11B depicts a representative flow cytometry assessment for TCR clone 01CA019_064_F05_0005. Shown are activation markers CD25 (left panels), CD69 (middle panels), and CD137 (right panels) in primary T cells transduced with the indicated TCR and stimulated with a cognate neoantigen (bottom panels) or corresponding wildtype peptide (top panels.

FIG. 12 depicts proliferation of primary T cells transduced with indicated candidate TCRs. Shown is the percentage of T cells with diluted CellTrace Violet dye following co-culture with peptide-loaded APCs.

### DETAILED DESCRIPTION

Unless otherwise defined, all terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, N.Y. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise. The terms “include,” “such as,” and the like are intended to convey inclusion without limitation, unless otherwise specifically indicated.

As used herein, the term “comprising” also specifically includes embodiments “consisting of” and “consisting essentially of” the recited elements, unless specifically indicated otherwise.

The term “about” indicates and encompasses an indicated value and a range above and below that value. In certain embodiments, the term “about” indicates the designated value ±10%, ±5%, or ±1%. In certain embodiments, where applicable, the term “about” indicates the designated value(s)±one standard deviation of that value(s).

The term “antigen binding protein” or “ABP” is used herein in its broadest sense and includes certain types of molecules comprising one or more antigen-binding domains that specifically bind to an antigen or epitope.

In some embodiments, the ABP comprises a TCR. In some embodiments, the ABP consists of a TCR. In some embodiments, the ABP consists essentially of a TCR. An ABP specifically includes intact TCR, TCR fragments, and ABP fragments. In some embodiments, the ABP comprises an alternative scaffold. In some embodiments, the ABP consists of an alternative scaffold. In some embodiments, the ABP consists essentially of an alternative scaffold. In some embodiments, the ABP comprises a TCRfragment. In some embodiments, the ABP consists of a TCRfragment. In some embodiments, the ABP consists essentially of a TCRfragment.

An “HLA-PEPTIDE ABP,” “anti-HLA-PEPTIDE ABP,” or “HLA-PEPTIDE-specific ABP” is an ABP, as provided herein, which specifically binds to the antigen HLA-PEPTIDE. An ABP includes proteins comprising one or more antigen-binding domains that specifically bind to an antigen or epitope via a variable region, such as a variable region derived from a T cell (e.g., a TCR).

As used herein, “variable region” refers to a variable sequence that arises from a recombination event, for example, it can include a V, J, and/or D segment of a T cell receptor (TCR) sequence from a T cell, such as an activated T cell.

The term “antigen-binding domain” means the portion of an ABP that is capable of specifically binding to an antigen or epitope. An antigen-binding domain can include TCR CDRs, e.g., αCDR1, αCDR2, αCDR3, βCDR1, βCDR2, and βCDR3. TCR CDRs are described herein.

The amino acid sequence boundaries of a TCR CDR can be determined by one of skill in the art using any of a number of known numbering schemes, including but not limited to the IMGT unique numbering, as described by LeFranc, M.-P, Immunol Today. 1997 November; 18(11):509; Lefranc, M.-P., “IMGT Locus on Focus: A new section of Experimental and Clinical Immunogenetics”, Exp. Clin. Immunogenet., 15, 1-7 (1998); Lefranc and Lefranc, The T Cell Receptor FactsBook; and M.-P. Lefranc/Developmental and Comparative Immunology 27 (2003) 55-77, all of which are incorporated by reference.

An “ABP fragment” comprises a portion of an intact ABP, such as the antigen-binding or variable region of an intact ABP. ABP fragments include, for example, TCR fragments.

The term “alternative scaffold” refers to a molecule in which one or more regions may be diversified to produce one or more antigen-binding domains that specifically bind to an antigen or epitope. In some embodiments, the antigen-binding domain binds the antigen or epitope with specificity and affinity similar to that of an ABP. Exemplary alternative scaffolds include those derived from fibronectin (e.g., Adnectins™), the β-sandwich (e.g., iMab), lipocalin (e.g., Anticalins®), EETI-II/AGRP, BPTI/LACI-D1/ITI-D2 (e.g., Kunitz domains), thioredoxin peptide aptamers, protein A (e.g., Affibody®), ankyrin repeats (e.g., DARPins), gamma-B-crystallin/ubiquitin (e.g., Affilins), CTLD₃ (e.g., Tetranectins), Fynomers, and (LDLR-A module) (e.g., Avimers). Additional information on alternative scaffolds is provided in Binz et al., Nat. Biotechnol., 2005 23: 1257-1268; Skerra, Current Opin. in Biotech., 2007 18:295-304; and Silacci et al., J. Biol. Chem., 2014, 289:14392-14398; each of which is incorporated by reference in its entirety. An alternative scaffold is one type of ABP.

“Affinity” refers to the strength of the sum total of non-covalent interactions between a single binding site of a molecule (e.g., an ABP) and its binding partner (e.g., an antigen or epitope). Unless indicated otherwise, as used herein, “affinity” refers to intrinsic binding affinity, which reflects a 1:1 interaction between members of a binding pair (e.g., ABP and antigen or epitope). The affinity of a molecule X for its partner Y can be represented by the dissociation equilibrium constant (K_{D}). The kinetic components that contribute to the dissociation equilibrium constant are described in more detail below. Affinity can be measured by common methods known in the art, including those described herein, such as surface plasmon resonance (SPR) technology (e.g., BIACORE®) or biolayer interferometry (e.g., FORTEBIO®).

With regard to the binding of an ABP to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the ABP to the target molecule is competitively inhibited by the control molecule.

In some aspects, the affinity of a HLA-PEPTIDE ABP for a non-target molecule is less than about 50% of the affinity for HLA-PEPTIDE. In some aspects, the affinity of a HLA-PEPTIDE ABP for a non-target molecule is less than about 40% of the affinity for HLA-PEPTIDE. In some aspects, the affinity of a HLA-PEPTIDE ABP for a non-target molecule is less than about 30% of the affinity for HLA-PEPTIDE. In some aspects, the affinity of a HLA-PEPTIDE ABP for a non-target molecule is less than about 20% of the affinity for HLA-PEPTIDE. In some aspects, the affinity of a HLA-PEPTIDE ABP for a non-target molecule is less than about 10% of the affinity for HLA-PEPTIDE. In some aspects, the affinity of a HLA-PEPTIDE ABP for a non-target molecule is less than about 1% of the affinity for HLA-PEPTIDE. In some aspects, the affinity of a HLA-PEPTIDE ABP for a non-target molecule is less than about 0.1% of the affinity for HLA-PEPTIDE.

The term “k_{d}” (sec⁻¹), as used herein, refers to the dissociation rate constant of a particular ABP—antigen interaction. This value is also referred to as the k_{off} value.

The term “kₐ” (M⁻¹×sec⁻¹), as used herein, refers to the association rate constant of a particular ABP-antigen interaction. This value is also referred to as the kₒₙ value.

The term “K_{D}” (M), as used herein, refers to the dissociation equilibrium constant of a particular ABP-antigen interaction. K_{D}=k_{d}/kₐ. In some embodiments, the affinity of an ABP is described in terms of the K_{D} for an interaction between such ABP and its antigen. For clarity, as known in the art, a smaller K_{D} value indicates a higher affinity interaction, while a larger K_{D} value indicates a lower affinity interaction.

The term “K_{A}” (M⁻¹), as used herein, refers to the association equilibrium constant of a particular ABP-antigen interaction. K_{A}=kₐ/k_{d}.

An “immunoconjugate” is an ABP conjugated to one or more heterologous molecule(s), such as a therapeutic (cytokine, for example) or diagnostic agent.

When used herein in the context of two or more ABPs, the term “competes with” or “cross-competes with” indicates that the two or more ABPs compete for binding to an antigen (e.g., HLA-PEPTIDE). In one exemplary assay, HLA-PEPTIDE is coated on a surface and contacted with a first HLA-PEPTIDE ABP, after which a second HLA-PEPTIDE ABP is added. In another exemplary assay, a first HLA-PEPTIDE ABP is coated on a surface and contacted with HLA-PEPTIDE, and then a second HLA-PEPTIDE ABP is added. If the presence of the first HLA-PEPTIDE ABP reduces binding of the second HLA-PEPTIDE ABP, in either assay, then the ABPs compete with each other. The term “competes with” also includes combinations of ABPs where one ABP reduces binding of another ABP, but where no competition is observed when the ABPs are added in the reverse order. However, in some embodiments, the first and second ABPs inhibit binding of each other, regardless of the order in which they are added. In some embodiments, one ABP reduces binding of another ABP to its antigen by at least 25%, at least 50%, at least 60%, at least 70%, at least 80%, at least 85%, at least 90%, or at least 95%. A skilled artisan can select the concentrations of the ABPs used in the competition assays based on the affinities of the ABPs for HLA-PEPTIDE and the valency of the ABPs. The assays described in this definition are illustrative, and a skilled artisan can utilize any suitable assay to determine if ABPs compete with each other. Suitable assays are described, for example, in Cox et al., “Immunoassay Methods,” in Assay Guidance Manual [Internet], Updated Dec. 24, 2014 (www.ncbi.nlm.nih.gov/books/NBK92434/; accessed Sep. 29, 2015); Silman et al., Cytometry, 2001, 44:30-37; and Finco et al., J. Pharm. Biomed. Anal., 2011, 54:351-358; each of which is incorporated by reference in its entirety.

The term “epitope” means a portion of an antigen that specifically binds to an ABP. Epitopes frequently consist of surface-accessible amino acid residues and/or sugar side chains and may have specific three dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. The epitope to which an ABP binds can be determined using known techniques for epitope determination such as, for example, testing for ABP binding to HLA-PEPTIDE variants with different point-mutations, or to chimeric HLA-PEPTIDE variants.

As used herein, the term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Alternatively, sequence similarity or dissimilarity can be established by the combined presence or absence of particular nucleotides, or, for translated sequences, amino acids at selected sequence positions (e.g., sequence motifs).

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

A “conservative substitution” or a “conservative amino acid substitution,” refers to the substitution an amino acid with a chemically or functionally similar amino acid. Conservative substitution tables providing similar amino acids are well known in the art. By way of example, the groups of amino acids provided in Tables 2-4 are, in some embodiments, considered conservative substitutions for one another.

TABLE 2
Selected groups of amino acids that are considered conservative
substitutions for one another, in certain embodiments.

| | |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

TABLE 3
Additional selected groups of amino acids that
are considered conservative substitutions
for one another, in certain embodiments.

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

TABLE 4
Further selected groups of amino acids that are considered conservative
substitutions for one another, in certain embodiments.

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2nd ed. (1993) W. H. Freeman & Co., New York, N.Y. An ABP generated by making one or more conservative substitutions of amino acid residues in a parent ABP is referred to as a “conservatively modified variant.”

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which an exogenous nucleic acid has been introduced, and the progeny of such cells. Host cells include “transformants” (or “transformed cells”) and “transfectants” (or “transfected cells”), which each include the primary transformed or transfected cell and progeny derived therefrom. Such progeny may not be completely identical in nucleic acid content to a parent cell, and may contain mutations.

The term “treating” (and variations thereof such as “treat” or “treatment”) refers to clinical intervention in an attempt to alter the natural course of a disease or condition in a subject in need thereof. Treatment can be performed both for prophylaxis and during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “therapeutically effective amount” or “effective amount” refers to an amount of an ABP or pharmaceutical composition provided herein that, when administered to a subject, is effective to treat a disease or disorder.

As used herein, the term “subject” means a mammalian subject. Exemplary subjects include humans, monkeys, dogs, cats, mice, rats, cows, horses, camels, goats, rabbits, and sheep. In certain embodiments, the subject is a human. In some embodiments the subject has a disease or condition that can be treated with an ABP provided herein. In some aspects, the disease or condition is a cancer. In some aspects, the disease or condition is a viral infection.

The term “package insert” is used to refer to instructions customarily included in commercial packages of therapeutic or diagnostic products (e.g., kits) that contain information about the indications, usage, dosage, administration, combination therapy, contraindications and/or warnings concerning the use of such therapeutic or diagnostic products.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein. The terms “cell proliferative disorder” and “proliferative disorder” refer to disorders that are associated with some degree of abnormal cell proliferation. In some embodiments, the cell proliferative disorder is a cancer. In some aspects, the tumor is a solid tumor. In some aspects, the tumor is a hematologic malignancy.

The term “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

The term “agonize” refers to the activation of receptor signaling to induce a biological response associated with activation of the receptor. An “agonist” is an entity that binds to and agonizes a receptor.

The term “antagonize” refers to the inhibition of receptor signaling to inhibit a biological response associated with activation of the receptor. An “antagonist” is an entity that binds to and antagonizes a receptor.

The terms “nucleic acids” and “polynucleotides” may be used interchangeably herein to refer to polymeric form of nucleotides of any length, either deoxyribonucleotides or ribonucleotides, or analogs thereof. Polynucleotides can include, but are not limited to coding or non-coding regions of a gene or gene fragment, loci (locus) defined from linkage analysis, exons, introns, messenger RNA (mRNA), cDNA, recombinant polynucleotides, branched polynucleotides, plasmids, vectors, isolated DNA, isolated RNA, nucleic acid probes, and primers. A polynucleotide may comprise modified nucleotides, such as methylated nucleotides and nucleotide analogs. Exemplary modified nucleotides include, e.g., 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthioN6- isopentenyladenine, uracil-5-oxyacetic acid (v), wybutoxosine, pseudouracil, queosine, 2- thiocytosine, 5-methyl-2-thiouracil, 2-thiouracil, 4-thiouracil, 5-methyluracil, uracil-5- oxyacetic acid methylester, 3-(3-amino-3-N-2-carboxypropyl) uracil, and 2,6-diaminopurine.

As used herein the term “antigen” is a substance that induces an immune response. An antigen can be a neoantigen. An antigen can be a “shared antigen” that is an antigen found among a specific population, e.g., a specific population of cancer patients. Antigens can include HLA-PEPTIDE antigens.

As used herein the term “neoantigen” is an antigen that has at least one alteration that makes it distinct from the corresponding wild-type antigen, e.g., via mutation in a tumor cell or post-translational modification specific to a tumor cell. In some embodiments, the alteration occurs in tumor or cancer cells. In some embodiments, the alteration does not occur in a non-tumor or a non-cancer cell. In some embodiments, the alteration is absent from normal tissue. A neoantigen can include a polypeptide sequence or a nucleotide sequence. A mutation can include a frameshift or nonframeshift indel, missense or nonsense substitution, splice site alteration, genomic rearrangement or gene fusion, or any genomic or expression alteration giving rise to a neoORF. A mutation can also include a splice variant. Post-translational modifications specific to a tumor cell can include aberrant phosphorylation.

Post-translational modifications specific to a tumor cell can also include a proteasome-generated spliced antigen. See Liepe et al., A large fraction of HLA class I ligands are proteasome-generated spliced peptides; Science. 2016 Oct. 21; 354(6310):354-358. A neoantigen can be a shared neoantigen if it can be found among multiple patients in a specific population (e.g., a specific population of cancer patients). Neoantigens can include HLA-PEPTIDE neoantigens.

As used herein, the terms “HLA-PEPTIDE,” “pHLA,” “peptide-HLA,” and “peptide-HLA complex,” are used interchangeably herein to refer to an antigen comprising an HLA-restricted peptide complexed with an HLA Class I molecule, wherein the HLA-restricted peptide is located in the peptide binding groove of an α1/α2 heterodimer portion of the HLA Class I molecule. Such antigens are defined by a specific HLA-restricted peptide having a defined amino acid sequence complexed with a specific HLA Class I subtype.

In some embodiments, an “HLA-PEPTIDE neoantigen,” a “pHLA neoantigen,” and a “peptide-HLA neoantigen” are used interchangeably herein to refer to an HLA-PEPTIDE that comprises at least one alteration that makes it distinct from the corresponding wild-type HLA-PEPTIDE antigen, e.g., via mutation in a tumor cell or post-translational modification specific to a tumor cell. In some embodiments, the at least one alteration is in the restricted peptide sequence, such that the restricted peptide of the HLA-PEPTIDE neoantigen is distinguished from a corresponding restricted peptide sequence without the alteration, e.g., a restricted peptide containing the wild-type sequence.

Exemplary HLA-PEPTIDE neoantigens and shared HLA-PEPTIDE neoantigens are shown in Table A (SEQ ID NO:10,755-21,015), in the AACR GENIE Results (SEQ ID NO:21,016-29,357), and in SEQ ID NOs 29358-29364; corresponding genes and somatic alterations associated with each antigen are also shown. Such pHLA neoantigens and shared pHLA neoantigens are useful for inducing an immune response in a subject via administration. The subject can be identified for administration through the use of various diagnostic methods, e.g., patient selection methods described herein.

As used herein the term “tumor antigen” is a antigen present in a subject's tumor cell or tissue but not in the subject's corresponding normal cell or tissue, or derived from a polypeptide known to or have been found to have altered expression in a tumor cell or cancerous tissue in comparison to a normal cell or tissue.

As used herein the term “candidate antigen” is a mutation or other aberration giving rise to a sequence that may represent an antigen.

As used herein the term “coding region” is the portion(s) of a gene that encode protein.

As used herein the term “coding mutation” is a mutation occurring in a coding region.

As used herein the term “ORF” means open reading frame.

As used herein the term “NEO-ORF” is a tumor-specific ORF arising from a mutation or other aberration such as splicing.

As used herein the term “missense mutation” is a mutation causing a substitution from one amino acid to another.

As used herein the term “nonsense mutation” is a mutation causing a substitution from an amino acid to a stop codon or causing removal of a canonical start codon.

As used herein the term “frameshift mutation” is a mutation causing a change in the frame of the protein.

As used herein the term “indel” is an insertion or deletion of one or more nucleic acids.

As used herein the term “non-stop or read-through” is a mutation causing the removal of the natural stop codon.

HLA-Peptide Antigens

The major histocompatibility complex (MHC) is a complex encoded by a group of linked loci, which are collectively termed H-2 in the mouse and HLA in humans. The two principal classes of the MHC antigens, class I and class II, each comprise a set of cell surface glycoproteins which play a role in determining tissue type and transplant compatibility. In transplantation reactions, cytotoxic T-cells (CTLs) respond mainly against class I glycoproteins, while helper T-cells respond mainly against class II glycoproteins.

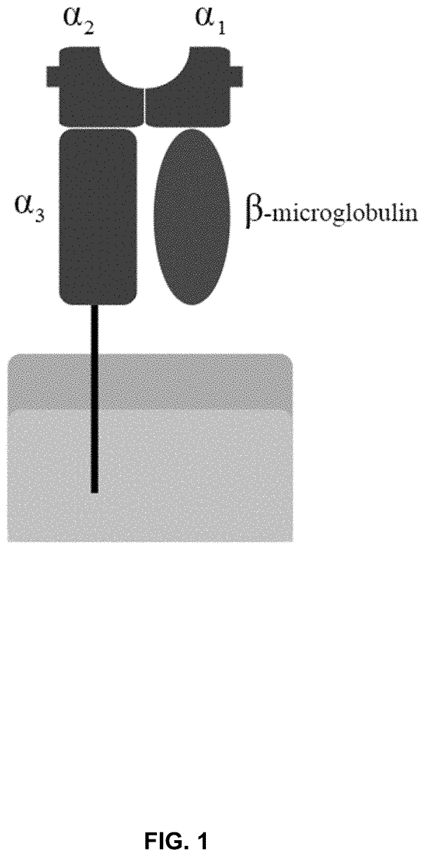

Human major histocompatibility complex (MHC) class I molecules, referred to interchangeably herein as HLA Class I molecules, are expressed on the surface of nearly all cells. These molecules function in presenting peptides which are mainly derived from endogenously synthesized proteins to, e.g., CD8+ T cells via an interaction with the alpha-beta T-cell receptor. The class I MHC molecule comprises a heterodimer composed of a 46-kDa α chain which is non-covalently associated with the 12-kDa light chain beta-2 microglobulin. The α chain generally comprises α1 and α2 domains which form a groove for presenting an HLA-restricted peptide, and an 03 plasma membrane-spanning domain which interacts with the CD8 co-receptor of T-cells. FIG. 1 depicts the general structure of a Class I HLA molecule. Some TCRs can bind MHC class I independently of CD8 coreceptor (see, e.g., Kerry S E, Buslepp J, Cramer L A, et al. Interplay between TCR Affinity and Necessity of Coreceptor Ligation: High-Affinity Peptide-MHC/TCR Interaction Overcomes Lack of CD8 Engagement. Journal of immunology (Baltimore, Md.: 1950). 2003; 171(9):4493-4503.)

Class I MHC-restricted peptides (also referred to interchangeably herein as HLA-restricted antigens, HLA-restricted peptides, antigenic peptides, MHC-restricted antigens, restricted peptides, or peptides) generally bind to the heavy chain alpha1-alpha2 groove via about two or three anchor residues that interact with corresponding binding pockets in the MHC molecule. The beta-2 microglobulin chain plays an important role in MHC class I intracellular transport, peptide binding, and conformational stability. For most class I molecules, the formation of a heterotrimeric complex of the MHC class I heavy chain, peptide (self, non-self, and/or antigenic) and beta-2 microglobulin leads to protein maturation and export to the cell-surface.

Binding of a given HLA subtype to an HLA-restricted peptide forms a complex with a unique and novel surface that can be specifically recognized by an ABP such as, e.g., a TCR on a T cell.

Accordingly, provided herein are HLA-PEPTIDE antigens comprising a specific HLA-restricted peptide having a defined amino acid sequence complexed with a specific HLA subtype.

HLA-PEPTIDE antigens identified herein may be useful for cancer immunotherapy. In some embodiments, the HLA-PEPTIDE antigens identified herein are presented on the surface of a tumor cell. The HLA-PEPTIDE antigens identified herein may be expressed by tumor cells in a human subject. The HLA-PEPTIDE antigens identified herein may be expressed by tumor cells in a population of human subjects. For example, the HLA-PEPTIDE antigens identified herein may be shared HLA-PEPTIDE antigens which are commonly expressed in a population of human subjects with cancer.

The HLA-PEPTIDE antigens identified herein may have a prevalence with an individual tumor type The prevalence with an individual tumor type may be about 0.1%, 0.2%0, 0.3%0, 0.4%0, 0.5%0, 0.6%0, 0.7%0, 0.8%0, 0.9%, 1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. The prevalence with an individual tumor type may be about 0.1%-100%, 0.2-50%, 0.5-25%, or 1-10%.

Exemplary HLA Class I Subtypes of the pHLA Neoantigens

In humans, there are many MHC haplotypes (referred to interchangeably herein as MHC subtypes, HLA subtypes, MHC types, and HLA types). Exemplary HLA subtypes include, by way of example only, 2 digit, 4 digit, 6 digit, and 8 digit subtypes. A full list of HLA Class Alleles can be found on http://hla.alleles.org/alleles/. For example, a full list of HLA Class I Alleles can be found on http://hla.alleles.org/alleles/class1.html. Exemplary HLA Class I subtypes include any of the HLA subtypes disclosed in in Table A (see SEQ ID NO:10,755-21,015) in the AACR GENIE results (see SEQ ID NO: 21,016-29,357), and in SEQ ID NOs: 29358-29364 disclosed herein. Table A neoantigens and the AACR GENIE Results are disclosed in PCT/US2019/033830, filed on May 23, 2019, which application is hereby incorporated by reference in its entirety.

Exemplary HLA-Restricted Peptides

The HLA-restricted peptides (referred to interchangeably herein) as “restricted peptides” can be peptide fragments of tumor-associated neoantigens, e.g., shared neoantigens. The peptide fragments can include any of the amino acid sequences disclosed in Table A (see SEQ ID NO:10,755-21,015), in the AACR GENIE results (see SEQ ID NO: 21,016-29,357), and in SEQ ID NOs: 29358-29364 disclosed herein. Table A neoantigens and the AACR GENIE Results are disclosed in PCT/US2019/033830, filed on May 23, 2019, which application is hereby incorporated by reference in its entirety.

Accordingly, disclosed herein are isolated peptides that comprise tumor specific mutations identified by the methods disclosed herein, peptides that comprise known tumor specific mutations, and mutant polypeptides or fragments thereof identified by methods disclosed herein. Neoantigen peptides can be described in the context of their coding sequence where a neoantigen includes the nucleotide sequence (e.g., DNA or RNA) that codes for the related polypeptide sequence.

Also disclosed herein are peptides, e.g., restricted peptides derived from any polypeptide known to or have been found to have altered expression in a tumor cell or cancerous tissue in comparison to a normal cell or tissue, for example any polypeptide known to or have been found to be aberrantly expressed in a tumor cell or cancerous tissue in comparison to a normal cell or tissue. Suitable polypeptides from which the restricted peptides can be derived can be found for example in the COSMIC database. COSMIC curates comprehensive information on somatic mutations in human cancer. In some embodiments, the restricted peptide contains the tumor specific mutation.

One or more restricted peptides can comprise at least one of a binding affinity with MHC with an IC50 value of less than 1000 nM, for MHC Class I peptides a length of 8-15, 8, 9, 10, 11, 12, 13, 14, or 15 amino acids, presence of sequence motifs within or near the peptide promoting proteasome cleavage, and presence or sequence motifs promoting TAP transport.

The restricted peptides may have a size of about 5, about 6, about 7, about 8, about 9, about 10, about 11, about 12, about 13, about 14, or about 15 amino acid residues, and any range derivable therein. In particular embodiments, the restricted peptide has a size of about 8, about 9, about 10, about 11, or about 12 amino molecule residues. The restricted peptide may be about 5-15 amino acids in length, preferably may be about 7-13 amino acids in length, or more preferably may be about 8-12 amino acids in length.

Exemplary Shared HLA-PEPTIDE Neoantigens

Exemplary shared HLA-PEPTIDE neoantigens are shown in Table A (see SEQ ID NO:10,755-21,015), in the AACR GENIE results (see SEQ ID NO: 21,016-29,357), and in SEQ ID NOs: 29358-29364 disclosed herein. Table A neoantigens and the AACR GENIE Results are disclosed in PCT/US2019/033830, filed on May 23, 2019, which application is hereby incorporated by reference in its entirety.

One or more HLA-PEPTIDE neoantigens can be presented on the surface of a tumor.

One or more HLA-PEPTIDE neoantigens can be immunogenic in a subject having a tumor, e.g., capable of eliciting a T cell response or a B cell response in the subject.

If desirable, a longer peptide can be designed in several ways. In one case, when presentation likelihoods of peptides on HLA alleles are predicted or known, a longer peptide could consist of either: (1) individual presented peptides with an extensions of 2-5 amino acids toward the N- and C-terminus of each corresponding gene product; (2) a concatenation of some or all of the presented peptides with extended sequences for each. In another case, when sequencing reveals a long (>10 residues) neoepitope sequence present in the tumor (e.g. due to a frameshift, read-through or intron inclusion that leads to a novel peptide sequence), a longer peptide would consist of: (3) the entire stretch of novel tumor-specific amino acids—thus bypassing the need for computational or in vitro test-based selection of the strongest HLA-presented shorter peptide. In both cases, use of a longer peptide allows endogenous processing by patient cells and may lead to more effective antigen presentation and induction of T cell responses.

Antigenic peptides and polypeptides can be presented on an HLA protein. In some aspects antigenic peptides and polypeptides are presented on an HLA protein with greater affinity than a wild-type peptide. In some aspects, a antigenic peptide or polypeptide can have an IC50 of at least less than 5000 nM, at least less than 1000 nM, at least less than 500 nM, at least less than 250 nM, at least less than 200 nM, at least less than 150 nM, at least less than 100 nM, at least less than 50 nM or less.

In some aspects, antigenic peptides and polypeptides do not induce an autoimmune response and/or invoke immunological tolerance when administered to a subject.

Also provided are compositions comprising at least two or more antigenic peptides. In some embodiments the composition contains at least two distinct peptides. At least two distinct peptides can be derived from the same polypeptide. By distinct polypeptides is meant that the peptide vary by length, amino acid sequence, or both. The peptides are derived from any polypeptide known to or have been found to contain a tumor specific mutation or peptides derived from any polypeptide known to or have been found to have altered expression in a tumor cell or cancerous tissue in comparison to a normal cell or tissue, for example any polypeptide known to or have been found to be aberrantly expressed in a tumor cell or cancerous tissue in comparison to a normal cell or tissue. Suitable polypeptides from which the antigenic peptides can be derived can be found for example in the COSMIC database or the AACR Genomics Evidence Neoplasia Information Exchange (GENIE) database. COSMIC curates comprehensive information on somatic mutations in human cancer. AACR GENIE aggregates and links clinical-grade cancer genomic data with clinical outcomes from tens of thousands of cancer patients. The peptide contains the tumor specific mutation. In some aspects the tumor specific mutation is a driver mutation for a particular cancer type.

Antigenic peptides and polypeptides having a desired activity or property can be modified to provide certain desired attributes, e.g., improved pharmacological characteristics, while increasing or at least retaining substantially all of the biological activity of the unmodified peptide to bind the desired MHC molecule and activate the appropriate T cell. For instance, antigenic peptide and polypeptides can be subject to various changes, such as substitutions, either conservative or non-conservative, where such changes might provide for certain advantages in their use, such as improved MHC binding, stability or presentation. By conservative substitutions is meant replacing an amino acid residue with another which is biologically and/or chemically similar, e.g., one hydrophobic residue for another, or one polar residue for another. The substitutions include combinations such as Gly, Ala; Val, Ile, Leu, Met; Asp, Glu; Asn, Gln; Ser, Thr; Lys, Arg; and Phe, Tyr. The effect of single amino acid substitutions may also be probed using D-amino acids. Such modifications can be made using well known peptide synthesis procedures, as described in e.g., Merrifield, Science 232:341-347 (1986), Barany & Merrifield, The Peptides, Gross & Meienhofer, eds. (N.Y., Academic Press), pp. 1-284 (1979); and Stewart & Young, Solid Phase Peptide Synthesis, (Rockford, Ill., Pierce), 2d Ed. (1984).

Modifications of peptides and polypeptides with various amino acid mimetics or unnatural amino acids can be particularly useful in increasing the stability of the peptide and polypeptide in vivo. Stability can be assayed in a number of ways. For instance, peptidases and various biological media, such as human plasma and serum, have been used to test stability. See, e.g., Verhoef et al., Eur. J. Drug Metab Pharmacokin. 11:291-302 (1986). Half-life of the peptides can be conveniently determined using a 25% human serum (v/v) assay. The protocol is generally as follows. Pooled human serum (Type AB, non-heat inactivated) is delipidated by centrifugation before use. The serum is then diluted to 25% with RPMI tissue culture media and used to test peptide stability. At predetermined time intervals a small amount of reaction solution is removed and added to either 6% aqueous trichloracetic acid or ethanol. The cloudy reaction sample is cooled (4 degrees C.) for 15 minutes and then spun to pellet the precipitated serum proteins. The presence of the peptides is then determined by reversed-phase HPLC using stability-specific chromatography conditions.

The peptides and polypeptides can be modified to provide desired attributes other than improved serum half-life. For instance, the ability of the peptides to induce CTL activity can be enhanced by linkage to a sequence which contains at least one epitope that is capable of inducing a T helper cell response. Immunogenic peptides/T helper conjugates can be linked by a spacer molecule. The spacer is typically comprised of relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need not be comprised of the same residues and thus can be a hetero- or homo-oligomer. When present, the spacer will usually be at least one or two residues, more usually three to six residues. Alternatively, the peptide can be linked to the T helper peptide without a spacer.

An antigenic peptide can be linked to the T helper peptide either directly or via a spacer either at the amino or carboxy terminus of the peptide. The amino terminus of either the antigenic peptide or the T helper peptide can be acylated. Exemplary T helper peptides include tetanus toxoid 830-843, influenza 307-319, malaria circumsporozoite 382-398 and 378-389.

Proteins or peptides can be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and can be found at computerized databases known to those of ordinary skill in the art. One such database is the National Center for Biotechnology Information's Genbank and GenPept databases located at the National Institutes of Health website. The coding regions for known genes can be amplified and/or expressed using the techniques disclosed herein or as would be known to those of ordinary skill in the art. Alternatively, various commercial preparations of proteins, polypeptides and peptides are known to those of skill in the art.

In some embodiments, an antigen can include a nucleic acid (e.g. polynucleotide) that encodes a antigenic peptide or portion thereof. The polynucleotide can be, e.g., DNA, cDNA, PNA, CNA, RNA (e.g., mRNA), either single- and/or double-stranded, or native or stabilized forms of polynucleotides, such as, e.g., polynucleotides with a phosphorothiate backbone, or combinations thereof and it may or may not contain introns.

A still further aspect provides an expression vector capable of expressing a polypeptide or portion thereof. Expression vectors for different cell types are well known in the art and can be selected without undue experimentation. Generally, DNA is inserted into an expression vector, such as a plasmid, in proper orientation and correct reading frame for expression. If necessary, DNA can be linked to the appropriate transcriptional and translational regulatory control nucleotide sequences recognized by the desired host, although such controls are generally available in the expression vector. The vector is then introduced into the host through standard techniques. Guidance can be found e.g. in Sambrook et al. (1989) Molecular Cloning, A Laboratory Manual, Cold Spring Harbor Laboratory, Cold Spring Harbor, N.Y.

HLA Class I molecules which do not associate with a restricted peptide ligand are generally unstable. Accordingly, the association of the restricted peptide with the α1/α2 groove of the HLA molecule may stabilize the non-covalent association of the β₂-microglobulin subunit of the HLA subtype with the α-subunit of the HLA subtype.

Stability of the non-covalent association of the β₂-microglobulin subunit of the HLA subtype with the α-subunit of the HLA subtype can be determined using any suitable means. For example, such stability may be assessed by dissolving insoluble aggregates of HLA molecules in high concentrations of urea (e.g., about 8M urea), and determining the ability of the HLA molecule to refold in the presence of the restricted peptide during urea removal, e.g., urea removal by dialysis. Such refolding approaches are described in, e.g., Proc. Natl. Acad. Sci. USA Vol. 89, pp. 3429-3433, April 1992, hereby incorporated by reference.

For other example, such stability may be assessed using conditional HLA Class I ligands. Conditional HLA Class I ligands are generally designed as short restricted peptides which stabilize the association of the β2 and a subunits of the HLA Class I molecule by binding to the α1/α2 groove of the HLA molecule, and which contain one or more amino acid modifications allowing cleavage of the restricted peptide upon exposure to a conditional stimulus. Upon cleavage of the conditional ligand, the β2 and α-subunits of the HLA molecule dissociate, unless such conditional ligand is exchanged for a restricted peptide which binds to the α1/α2 groove and stabilizes the HLA molecule. Conditional ligands can be designed by introducing amino acid modifications in either known HLA peptide ligands or in predicted high-affinity HLA peptide ligands. For HLA alleles for which structural information is available, water-accessibility of side chains may also be used to select positions for introduction of the amino acid modifications. Use of conditional HLA ligands may be advantageous by allowing the batch preparation of stable HLA-peptide complexes which may be used to interrogate test restricted peptides in a high throughput manner. Conditional HLA Class I ligands, and methods of production, are described in, e.g., Proc Natl Acad Sci USA. 2008 Mar. 11; 105(10): 3831-3836; Proc Natl Acad Sci USA. 2008 Mar. 11; 105(10): 3825-3830; J Exp Med. 2018 May 7; 215(5): 1493-1504; Choo, J. A. L. et al. Bioorthogonal cleavage and exchange of major histocompatibility complex ligands by employing azobenzene-containing peptides. Angew Chem Int Ed Engl 53, 13390-13394 (2014); Amore, A. et al. Development of a Hypersensitive Periodate-Cleavable Amino Acid that is Methionine- and Disulfide-Compatible and its Application in MHC Exchange Reagents for T Cell Characterisation. ChemBioChem 14, 123-131 (2012); Rodenko, B. et al. Class I Major Histocompatibility Complexes Loaded by a Periodate Trigger. J Am Chem Soc 131, 12305-12313 (2009); and Chang, C. X. L. et al. Conditional ligands for Asian HLA variants facilitate the definition of CD8+ T-cell responses in acute and chronic viral diseases. Eur J Immunol 43, 1109-1120 (2013). These references are incorporated by reference in their entirety.

Accordingly, in some embodiments, the ability of an HLA-restricted peptide described herein, e.g., described in Table A (SEQ ID NO:10,755-21,015), AACR GENIE results (SEQ ID NOs: 21,016-29,357), or in SEQ ID NOs: 29358-29364, to stabilize the association of the β₂- and α-subunits of the HLA molecule, is assessed by performing a conditional ligand mediated-exchange reaction and assay for HLA stability. HLA stability can be assayed using any suitable method, including, e.g., mass spectrometry analysis, immunoassays (e.g., ELISA), size exclusion chromatography, and HLA multimer staining followed by flow cytometry assessment of T cells.

Other exemplary methods for assessing stability of the non-covalent association of the β₂-microglobulin subunit of the HLA subtype with the α-subunit of the HLA subtype include peptide exchange using dipeptides. Peptide exchange using dipeptides has been described in, e.g., Proc Natl Acad Sci USA. 2013 Sep. 17, 110(38):15383-8; Proc Natl Acad Sci USA. 2015 Jan. 6, 112(1):202-7, which is hereby incorporated by reference.

The HLA-PEPTIDE antigen may be isolated and/or in substantially pure form. For example, the HLA-PEPTIDE antigens may be isolated from their natural environment, or may be produced by means of a technical process. In some cases, the HLA-PEPTIDE antigen is provided in a form which is substantially free of other peptides or proteins.

The HLA-PEPTIDE antigens may be presented in soluble form, and optionally may be a recombinant HLA-PEPTIDE antigen complex. The skilled artisan may use any suitable method for producing and purifying recombinant HLA-PEPTIDE antigens. Suitable methods include, e.g., use of E. coli expression systems, insect cells, and the like. Other methods include synthetic production, e.g., using cell free systems. An exemplary suitable cell free system is described in WO2017089756, which is hereby incorporated by reference in its entirety.

Also provided herein are compositions comprising an HLA-PEPTIDE antigen.

In some cases, the composition comprises an HLA-PEPTIDE antigen attached to a solid support. Exemplary solid supports include, but are not limited to, beads, wells, membranes, tubes, columns, plates, sepharose, magnetic beads, and chips. Exemplary solid supports are described in, e.g., Catalysts 2018, 8, 92; doi:10.3390/cata18020092, which is hereby incorporated by reference in its entirety.

The HLA-PEPTIDE antigen may be attached to the solid support by any suitable methods known in the art. In some cases, the HLA-PEPTIDE antigen is covalently attached to the solid support.

In some cases, the HLA-PEPTIDE antigen is attached to the solid support by way of an affinity binding pair. Affinity binding pairs generally involved specific interactions between two molecules. A ligand having an affinity for its binding partner molecule can be covalently attached to the solid support, and thus used as bait for immobilizing. Common affinity binding pairs include, e.g., streptavidin and biotin, avidin and biotin; polyhistidine tags with metal ions such as copper, nickel, zinc, and cobalt; and the like. Accordingly, provided herein are compositions comprising an HLA-PEPTIDE antigen disclosed herein, wherein the HLA-PEPTIDE antigen is covalently linked to an affinity tag.

The HLA-PEPTIDE antigen may comprise a detectable label. In some embodiments, the HLA-PEPTIDE antigen is complexed with the detectable label. In some embodiments, the detectable label comprises a β₂-microglobulin binding molecule. e.g., a labeled antibody, e.g., a fluorochrome labeled antibody.

Also provided herein are pharmaceutical compositions comprising HLA-PEPTIDE antigens.

The composition comprising an HLA-PEPTIDE antigen may be a pharmaceutical composition. Such a composition may comprise multiple HLA-PEPTIDE antigens.

Exemplary pharmaceutical compositions are described herein. The composition may be capable of eliciting an immune response. The composition may comprise an adjuvant. Suitable adjuvants include, but are not limited to 1018 ISS, alum, aluminium salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel vector system, PLG microparticles, resiquimod, SRL172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Adjuvants such as incomplete Freund's or GM-CSF are useful. Several immunological adjuvants (e.g., MF59) specific for dendritic cells and their preparation have been described previously (Dupuis M, et al., Cell Immunol. 1998; 186(1):18-27; Allison A C; Dev Biol Stand. 1998; 92:3-11). Also cytokines can be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al., J Immunother Emphasis Tumor Immunol. 1996 (6):414-418). HLA surface expression and processing of intracellular proteins into peptides to present on HLA can also be enhanced by interferon-gamma (IFN-γ). See, e.g., York I A, Goldberg A L, Mo X Y, Rock K L. Proteolysis and class I major histocompatibility complex antigen presentation. Immunol Rev. 1999; 172:49-66; and Rock K L, Goldberg A L. Degradation of cell proteins and the generation of MHC class I-presented peptides. Ann Rev Immunol. 1999; 17:12. 739-779, which are incorporated herein by reference in their entirety.

Also provided herein are host cells comprising an HLA-PEPTIDE antigen disclosed herein. In some embodiments, the host cell comprises a polynucleotide encoding an HLA-restricted peptide as defined by the HLA-PEPTIDE antigen. In some embodiments, the polynucleotide is heterologous to the host cell. In some embodiments, the host cell does not comprise endogenous MHC. In some embodiments, the host cell comprises an exogenous HLA Class I molecule. In some embodiments, the host cell is a K562 or A375 cell. In some embodiments, the host cell is a cultured cell from a tumor cell line. In some embodiments, the tumor cell line expresses an HLA subtype as defined by the HLA-PEPTIDE antigen.

Also provided herein are cell culture systems comprising a host cell disclosed herein and a cell culture medium. In some embodiments, the host cell expresses the HLA Class I subtype as defined by the HLA-PEPTIDE antigen and the cell culture medium comprises the restricted peptide as defined by the HLA-PEPTIDE antigen.

ABPs

Also provided herein are ABPs that specifically bind to an HLA-PEPTIDE antigen disclosed herein. In some embodiments, an ABP disclosed herein specifically binds to an HLA-PEPTIDE neoantigen comprising an HLA-restricted peptide complexed with an HLA Class I molecule, wherein the HLA-restricted peptide is located in the peptide binding groove of an α1/α2 heterodimer portion of the HLA Class I molecule, wherein the HLA Class I molecule and the HLA-restricted peptide are each selected from an HLA-PEPTIDE neoantigen as described in any one of SEQ ID NOs:10,755 to 29,364, and wherein the ABP comprises a TCR or antigen-binding fragment thereof. For example, for an ABP disclosed herein, the target of the ABP is an HLA Class I molecule and the associated HLA-restricted peptide that are each selected from a single HLA-PEPTIDE neoantigen described in any one of the aforementioned SEQ ID NOs, i.e., the HLA Class I molecule and the HLA-restricted peptide are each selected from the same SEQ ID NO. For example, the target of an ABP against SEQ ID NO: 19865 would bind to HLA-A*11:01 in complex with a restricted peptide of the sequence: VVVGADGVGK.

The HLA-PEPTIDE neoantigen may be expressed on the surface of any suitable target cell including a tumor cell.

In some embodiments, the ABP specifically binds a complex comprising HLA and an HLA-restricted peptide (HLA-PEPTIDE), e.g., derived from a tumor. In some embodiments, the ABP does not bind to the HLA in the absence of the HLA-restricted peptide. In some embodiments, the ABP does not bind HLA-restricted peptide in the absence of the HLA. In some embodiments, the ABP binds tumor cells presenting human MHC complexed with the HLA—restricted peptide, optionally wherein the HLA restricted peptide is a tumor antigen characterizing the cancer. In some aspects, the ABP binds a complex comprising HLA and HLA-restricted peptide when naturally presented on a cell such as a tumor cell.

An ABP can bind to each portion of an HLA-PEPTIDE complex (i.e., HLA and peptide representing each portion of the complex), which when bound together form a novel target and protein surface for interaction with and binding by the ABP, distinct from a surface presented by the peptide alone or HLA subtype alone. Generally the novel target and protein surface formed by binding of HLA to peptide does not exist in the absence of each portion of the HLA-PEPTIDE complex. In some embodiments, the ABP binds to the HLA-PEPTIDE neoantigen through at least one contact point with the HLA Class I molecule and through at least one contact point with the HLA-restricted peptide.

In some embodiments, an ABP provided herein modulates binding of HLA-PEPTIDE to one or more ligands of HLA-PEPTIDE.

In more particular embodiments, the ABP specifically binds to a neoantigen described in Table 5A. In more particular embodiments, the ABP specifically binds to a neoantigen described in Table 5B. In more particular embodiments, the ABP specifically binds to a neoantigen described in Table 6. In more particular embodiments, the ABP specifically binds to a neoantigen described in Table 7.

In some embodiments of the ABP, the HLA-restricted peptide comprises a RAS mutation. In some embodiments of the ABP, the RAS mutation is a RAS G12 mutation. The RAS may be KRAS, NRAS, or HRAS. In some embodiments of the ABP, the HLA-restricted peptide comprises a RAS G12 mutation. In some embodiments of the ABP, the HLA-restricted peptide comprises a NRAS G12 mutation. In some embodiments of the ABP, the HLA-restricted peptide comprises a HRAS G12 mutation. Because amino acid positions 1-50 of HRAS, KRAS, and NRAS are identical, a skilled artisan understands that an HLA-Class I restricted peptide comprising a RAS G12 mutation corresponds to the KRAS G12, NRAS G12, and HRAS G12 mutation. By way of example only, SEQ ID NO: 14954, described as a KRAS G12C neoantigen, and SEQ ID NO: 14955, described as an NRAS G12C neoantigen, both have identical HLA-PEPTIDE pairings (HLA-A*02:01_KLVVVGACGV). Accordingly, SEQ ID NOs 14954 and 14955 describe identical KRAS/NRAS/HRAS G12C HLA-PEPTIDE neoantigens.

In some embodiments, the G12 mutation is a G12C, a G12D, a G12V, or a G12A mutation. In some embodiments wherein the HLA-restricted peptide comprises the RAS G12 mutation, the HLA Class I molecule is selected from HLA-A*02:01, HLA-A*11:01, HLA-A*31:01, HLA-C*01:02, and HLA-A*03:01.

In particular embodiments of the ABP, the HLA-PEPTIDE neoantigen is selected from: a RAS_G12C MHC Class I antigen comprising HLA-A*02:01 and the restricted peptide Lys Leu Val Val Val Gly Ala Cys Gly Val; a RAS_G12D MHC Class I antigen comprising HLA-A*11:01 and the restricted peptide Val Val Val Gly Ala Asp Gly Val Gly Lys; a RAS_G12D MHC Class I antigen comprising HLA-A*11:01 and the restricted peptide Val Val Gly Ala Asp Gly Val Gly Lys; a RAS_G12V MHC Class I antigen comprising HLA-A*11:01 and the restricted peptide Val Val Val Gly Ala Val Gly Val Gly Lys; a RAS_G12V MHC Class I antigen comprising HLA-A*31:01 and the restricted peptide Val Val Val Gly Ala Val Gly Val Gly Lys; a RAS_G12V MHC Class I antigen comprising HLA-A*11:01 and the restricted peptide Val Val Gly Ala Val Gly Val Gly Lys; a RAS_G12V MHC Class I antigen comprising HLA-C*01:02 and the restricted peptide Ala Val Gly Val Gly Lys Ser Ala Leu; and a RAS_G12V MHC Class I antigen comprising HLA-A*03:01 and the restricted peptide Val Val Val Gly Ala Val Gly Val Gly Lys.

In some embodiments of the ABP, the HLA-PEPTIDE neoantigen is selected from: a RAS_G12C MHC Class I antigen comprising HLA-A*02:01 and the restricted peptide Lys Leu Val Val Val Gly Ala Cys Gly Val; a RAS_G12D MHC Class I antigen comprising HLA-A*11:01 and the restricted peptide Val Val Val Gly Ala Asp Gly Val Gly Lys; a RAS_G12D MHC Class I antigen comprising HLA-A*11:01 and the restricted peptide Val Val Gly Ala Asp Gly Val Gly Lys; a RAS_G12V MHC Class I antigen comprising HLA-A*11:01 and the restricted peptide Val Val Val Gly Ala Val Gly Val Gly Lys; a RAS_G12V MHC Class I antigen comprising HLA-A*31:01 and the restricted peptide Val Val Val Gly Ala Val Gly Val Gly Lys; a RAS_G12V MHC Class I antigen comprising HLA-A*11:01 and the restricted peptide Val Val Gly Ala Val Gly Val Gly Lys; a RAS_G12V MHC Class I antigen comprising HLA-C*01:02 and the restricted peptide Ala Val Gly Val Gly Lys Ser Ala Leu; and a RAS_G12V MHC Class I antigen comprising HLA-A*03:01 and the restricted peptide Val Val Val Gly Ala Val Gly Val Gly Lys.

In some embodiments of the ABP, the HLA-PEPTIDE neoantigen is selected from: a RAS_G12C MHC Class I antigen comprising HLA-A*02:01 and the restricted peptide Lys Leu Val Val Val Gly Ala Cys Gly Val; a RAS_G12D MHC Class I antigen comprising HLA-A*11:01 and the restricted peptide Val Val Val Gly Ala Asp Gly Val Gly Lys; and a RAS_G12V MHC Class I antigen comprising HLA-A*11:01 and the restricted peptide Val Val Val Gly Ala Val Gly Val Gly Lys. In some embodiments of the ABP, the antigen comprises HLA-A*02:01 and the restricted peptide Lys Leu Val Val Val Gly Ala Cys Gly Val. In some embodiments of the ABP, the antigen comprises HLA-A*11:01 and the restricted peptide Val Val Val Gly Ala Asp Gly Val Gly Lys. In some embodiments of the ABP, the antigen comprises HLA-A*11:01 and the restricted peptide Val Val Val Gly Ala Val Gly Val Gly Lys.

In some embodiments of an ABP that binds to a RAS_G12C MHC Class I antigen comprising HLA-A*02:01 and the restricted peptide Lys Leu Val Val Val Gly Ala Cys Gly Val, the ABP binds to such RAS_G12 MHC Class I antigen at a higher affinity than a RAS_G12C MHC Class I antigen comprising the restricted peptide Lys Leu Val Val Val Gly Ala Cys Gly Val and a different HLA subtype. In some embodiments, the ABP binds to such RAS_G12 MHC Class I antigen at a higher affinity than a RAS_G12C MHC Class I antigen comprising the restricted peptide Lys Leu Val Val Val Gly Ala Cys Gly Val and a different HLA-A2 subtype. In some embodiments, the ABP does not bind to a RAS_G12C MHC Class I antigen comprising the restricted peptide Lys Leu Val Val Val Gly Ala Cys Gly Val and a different HLA-A2 subtype.

In some embodiments of an ABP that binds to an antigen comprising a particular RAS G12 mutation, the ABP does not binds to the particular antigen at a lower affinity than an antigen comprising a different RAS G12 mutation. For example, an ABP that binds to a RAS_G12C MHC Class I antigen comprising HLA-A*02:01 and the restricted peptide Lys Leu Val Val Val Gly Ala Cys Gly Val does not bind to that RAS_G12C MHC Class I antigen at a lower affinity than an antigen comprising a different RAS G12 mutation. In some embodiments of an ABP that binds to an antigen comprising a particular RAS G12 mutation, the ABP binds to the particular antigen at a higher affinity than an antigen comprising a different RAS G12 mutation. For example, an ABP that binds to a RAS_G12C MHC Class I antigen comprising HLA-A*02:01 and the restricted peptide Lys Leu Val Val Val Gly Ala Cys Gly Val may bind to that RAS_G12C MHC Class I antigen at a higher affinity than an antigen comprising a different RAS G12 mutation. In some embodiments, the ABP binds a RAS_G12C MHC Class I antigen comprising HLA-A*02:01 and the restricted peptide Lys Leu Val Val Val Gly Ala Cys Gly Val at a higher affinity than an antigen comprising the restricted peptide KLVVVGAVGV and an HLA-A2 molecule. In particular embodiments, such ABP does not bind to an antigen comprising the restricted peptide KLVVVGAVGV and an HLA-A2 molecule.

In some embodiments, the higher affinity is at least 2-fold, at least 5-fold, or at least 10-fold.

Affinity differences can be determined by any means known in the art. In some embodiments, such affinity differences are assessed by MSD-ECL, SPR, BLI, or flow cytometry.

In some embodiments, an ABP is an ABP that competes with an illustrative ABP provided herein. In some aspects, the ABP that competes with the illustrative ABP provided herein binds the same epitope as an illustrative ABP provided herein.

In some embodiments, the ABPs described herein are referred to herein as “variants.” In some embodiments, a variant is derived from any of the sequences provided herein, wherein one or more conservative amino acid substitutions are made. Conservative amino acid substitutions are described herein. In preferred embodiments, the non-conservative amino acid substitution does not interfere with or inhibit the biological activity of the functional variant. In yet more preferred embodiments, the non-conservative amino acid substitution enhances the biological activity of the functional variant, such that the biological activity of the functional variant is increased as compared to the parent ABP.

TCRs

In an aspect, the ABPs provided herein, e.g., ABPs that specifically bind HLA-PEPTIDE targets disclosed herein, include T cell receptors (TCRs). The TCRs may be isolated and purified.

In a majority of T-cells, the TCR is a heterodimer polypeptide having an alpha (α) chain and beta-(β) chain, encoded by TRA and TRB, respectively. The alpha chain generally comprises an alpha variable region, encoded by TRAV, an alpha joining region, encoded by TRAJ, and an alpha constant region, encoded by TRAC. The beta chain generally comprises a beta variable region, encoded by TRBV, a beta diversity region, encoded by TRBD, a beta joining region, encoded by TRBJ, and a beta constant region, encoded by TRBC. The TCR-alpha chain is generated by VJ recombination of alpha V and J segments, and the beta chain receptor is generated by V(D)J recombination of beta V, D, and J segments. Additional TCR diversity stems from junctional diversity. Several bases may be deleted and others added (called N and P nucleotides) at each of the junctions. In a minority of T-cells, the TCRs include gamma and delta chains. The TCR gamma chain is generated by VJ recombination, and the TCR delta chain is generated by V(D)J recombination (Kenneth Murphy, Paul Travers, and Mark Walport, Janeway's Immunology 7th edition, Garland Science, 2007, which is herein incorporated by reference in its entirety). The antigen binding site of a TCR generally comprises six complementarity determining regions (CDRs). The alpha chain contributes three CDRs, alpha (“α”) CDR1, αCDR2, and αCDR3. The beta chain also contributes three CDR: beta (“β”) CDR1, βCDR2, and βCDR3. In general, the αCDR3 and βCDR3 are the regions most affected by V(D)J recombination and account for most of the variation in a TCR repertoire.

TCRs can specifically recognize HLA-PEPTIDE targets, such as an HLA-PEPTIDE target disclosed in Table 7, Table A, the AACR GENIE Results, or SEQ ID NOs 29358-29364 described herein (SEQ ID NOs: 10,755-29,364); thus TCRs can be ABPs that specifically bind to HLA-PEPTIDE. TCRs can be soluble, e.g., similar to an antibody secreted by a B cell. TCRs can also be membrane-bound, e.g., on a cell such as a T cell or natural killer (NK) cell. Thus, TCRs can be used in a context that corresponds to soluble antibodies and/or membrane-bound CARs.

Any of the TCRs disclosed herein may comprise an alpha variable (“V”) segment, an alpha joining (“J”) segment, optionally an alpha constant region, a beta variable (“V”) segment, optionally a beta diversity (“D”) segment, a beta joining (“J”) segment, and optionally a beta constant region.

In some embodiments, the TCR or CAR is a recombinant TCR or CAR. The recombinant TCR or CAR may include any of the TCRs identified herein but include one or more modifications. Exemplary modifications, e.g., amino acid substitutions, are described herein. Amino acid substitutions described herein may be made with reference to IMGT nomenclature and amino acid numbering as found at www.imgt.org.

The recombinant TCR or CAR may be a human TCR or CAR, comprising fully human sequences, e.g., natural human sequences. The recombinant TCR or CAR may retain its natural human variable domain sequences but contain modifications to the a constant region, β constant region, or both α and β constant regions. Such modifications to the TCR constant regions may improve TCR assembly and expression for TCR gene therapy by, e.g., driving preferential pairings of the exogenous TCR chains.

In some embodiments, the α and β constant regions are modified by substituting the entire human constant region sequences for mouse constant region sequences. Such “murinized” TCRs and methods of making them are described in Cancer Res. 2006 Sep. 1; 66(17):8878-86, which is hereby incorporated by reference in its entirety.

In some embodiments, the α and β constant regions are modified by making one or more amino acid substitutions in the human TCR α constant (TRAC) region, the TCR β constant (TRBC) region, or the TRAC and TRAB regions, which swap particular human residues for murine residues (human→murine amino acid exchange). The one or more amino acid substitutions in the TRAC region may include a Ser substitution at residue 90, an Asp substitution at residue 91, a Val substitution at residue 92, a Pro substitution at residue 93, or any combination thereof. The one or more amino acid substitutions in the human TRBC region may include a Lys substitution at residue 18, an Ala substitution at residue 22, an Ile substitution at residue 133, a His substitution at residue 139, or any combination of the above. Such targeted amino acid substitutions are described in J Immunol Jun. 1, 2010, 184 (11) 6223-6231, which is hereby incorporated by reference in its entirety.

In some embodiments, the human TRAC contains an Asp substitution at residue 210 and the human TRBC contains a Lys substitution at residue 134. Such substitutions may promote the formation of a salt bridge between the alpha and beta chains and formation of the TCR interchain disulfide bond. These targeted substitutions are described in J Immunol Jun. 1, 2010, 184 (11) 6232-6241, which is hereby incorporated by reference in its entirety.

In some embodiments, the human TRAC and human TRBC regions are modified to contain introduced cysteines which may improve preferential pairing of the exogenous TCR chains through formation of an additional disulfide bond. For example, the human TRAC may contain a Cys substitution at residue 48 and the human TRBC may contain a Cys substitution at residue 57, described in Cancer Res. 2007 Apr. 15; 67(8):3898-903 and Blood. 2007 Mar. 15; 109(6):2331-8, which are hereby incorporated by reference in their entirety.

The recombinant TCR or CAR may comprise other modifications to the α and 3 chains.

In some embodiments, the α and β chains are modified by linking the extracellular domains of the α and β chains to a complete human CD3((CD3-zeta) molecule. Such modifications are described in J Immunol Jun. 1, 2008, 180 (11) 7736-7746; Gene Ther. 2000 August; 7(16):1369-77; and The Open Gene Therapy Journal, 2011, 4: 11-22, which are hereby incorporated by reference in their entirety.

In some embodiments, the α chain is modified by introducing hydrophobic amino acid substitutions in the transmembrane region of the α chain, as described in J Immunol Jun. 1, 2012, 188 (11) 5538-5546; hereby incorporated by reference in their entirety.

The alpha or beta chain may be modified by altering any one of the N-glycosylation sites in the amino acid sequence, as described in J Exp Med. 2009 Feb. 16; 206(2): 463-475; hereby incorporated by reference in its entirety.

The alpha and beta chain may each comprise a dimerization domain, e.g., a heterologous dimerization domain. Such a heterologous domain may be a leucine zipper, a 5H3 domain or hydrophobic proline rich counter domains, or other similar modalities, as known in the art. In one example, the alpha and beta chains may be modified by introducing 30mer segments to the carboxyl termini of the alpha and beta extracellular domains, wherein the segments selectively associate to form a stable leucine zipper. Such modifications are described in PNAS Nov. 22, 1994. 91 (24) 11408-11412; https://doi.org/10.1073/pnas. 91.24.11408; hereby incorporated by reference in its entirety.

TCRs identified herein may be modified to include mutations that result in increased affinity or half-life, such as those described in WO2012/013913, hereby incorporated by reference in its entirety.

The recombinant TCR or CAR may be a single chain TCR (scTCR). Such scTCR may comprise an α chain variable region sequence fused to the N terminus of a TCR α chain constant region extracellular sequence, a TCR β chain variable region fused to the N terminus of a TCR β chain constant region extracellular sequence, and a linker sequence linking the C terminus of the a segment to the N terminus of the β segment, or vice versa. In some embodiments, the constant region extracellular sequences of the α and β segments of the scTCR are linked by a disulfide bond. In some embodiments, the length of the linker sequence and the position of the disulfide bond being such that the variable region sequences of the α and β segments are mutually orientated substantially as in native a T cell receptors. Exemplary scTCRs are described in U.S. Pat. No. 7,569,664, which is hereby incorporated by reference in its entirety.

In some cases, the variable regions of the scTCR may be covalently joined by a short peptide linker, such as described in Gene Therapy volume 7, pages 1369-1377 (2000). The short peptide linker may be a serine rich or glycine rich linker. For example, the linker may be (Gly₄Ser)₃, as described in Cancer Gene Therapy (2004) 11, 487-496, incorporated by reference in its entirety.

The recombinant TCR or antigen binding fragment thereof may be expressed as a fusion protein. For instance, the TCR or antigen binding fragment thereof may be fused with a toxin. Such fusion proteins are described in Cancer Res. 2002 Mar. 15; 62(6):1757-60. The TCR or antigen binding fragment thereof may be fused with an antibody Fc region. Such fusion proteins are described in J Immunol May 1, 2017, 198 (1 Supplement) 120.9.

The antigen recognition domain of a receptor such as a TCR or CAR can be linked to one or more intracellular signaling components, such as signaling components that mimic activation through an antigen receptor complex, such as a TCR complex and/or signal via another cell surface receptor. For example, the HLA-PEPTIDE-specific binding component (e.g., an ABP such as a TCR) can be linked to one or more transmembrane and/or intracellular signaling domains. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CDS, CD9, CD 16, CD22, CD33, CD37, CD64, CD80, CD86, CD 134, CD137, and/or CD 154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the receptor.

The receptor, e.g., the TCR or CAR, can include at least one intracellular signaling component or components. In some embodiments, the receptor includes an intracellular component of a TCR complex, such as a TCR CD3 chain that mediates T-cell activation and cytotoxicity, e.g., CD3 zeta chain. For example, the HLA-PEPTIDE-binding ABP (e.g., a TCR or CAR) is linked to one or more cell signaling modules. In some embodiments, cell signaling modules include CD3 transmembrane domain, CD3 intracellular signaling domains, and/or other CD transmembrane domains. In some embodiments, the receptor, e.g., a TCR or CAR, further includes a portion of one or more additional molecules such as Fc receptor-gamma, CD8, CD4, CD25, or CD16. For example, in some aspects, the TCR or CAR includes a chimeric molecule between CD3-zeta or Fc receptor-gamma and CD8, CD4, CD25 or CD16.

In some embodiments, upon ligation of the TCR or CAR, the cytoplasmic domain or intracellular signaling domain of the receptor activates at least one of the normal effector functions or responses of the immune cell, e.g., T cell engineered to express the receptor. For example, in some contexts, the receptor induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the receptor. In other embodiments, the receptor does not include a component for generating a costimulatory signal. In some aspects, an additional receptor is expressed in the same cell and provides the component for generating the secondary or costimulatory signal.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the receptor includes one or both of such signaling components.

In some aspects, the receptor includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, FcR gamma, FcR beta, CD3 gamma, CD3 delta, CD3 epsilon, CDS, CD22, CD79a, CD79b, and CD66d. In some embodiments, cytoplasmic signaling molecule(s) in the CAR contain(s) a cytoplasmic signaling domain, portion thereof, or sequence derived from CD3 zeta.

In some embodiments, the receptor includes a signaling domain and/or transmembrane portion of a costimulatory receptor, such as CD28, 4-1BB, OX40, DAP10, and ICOS. In some aspects, the same receptor includes both the activating and costimulatory components.

In some embodiments, the activating domain is included within one receptor, whereas the costimulatory component is provided by another receptor recognizing another antigen. In some embodiments, the receptors include activating or stimulatory receptors, and costimulatory receptors, both expressed on the same cell (see WO2014/055668). In some aspects, the HLA-PEPTIDE-targeting receptor is the stimulatory or activating receptor; in other aspects, it is the costimulatory receptor. In some embodiments, the cells further include inhibitory receptors (e.g., iCARs, see Fedorov et al., Sci. Transl. Medicine, 5(215) (December, 2013), such as a receptor recognizing an antigen other than HLA-PEPTIDE, whereby an activating signal delivered through the HLA-PEPTIDE-targeting receptor is diminished or inhibited by binding of the inhibitory receptor to its ligand, e.g., to reduce off-target effects.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the receptor encompasses one or more, e.g., two or more, costimulatory domains and an activation domain, e.g., primary activation domain, in the cytoplasmic portion. Exemplary receptors include intracellular components of CD3-zeta, CD28, and 4-1BB.

In some embodiments, the CAR (or other antigen receptor such as a TCR) further includes a marker, such as a cell surface marker, which may be used to confirm transduction or engineering of the cell to express the receptor, such as a truncated version of a cell surface receptor, such as truncated EGFR (tEGFR). In some aspects, the marker includes all or part (e.g., truncated form) of CD34, a nerve growth factor receptor (NGFR), or epidermal growth factor receptor (e.g., tEGFR). In some embodiments, the nucleic acid encoding the marker is operably linked to a polynucleotide encoding for a linker sequence, such as a cleavable linker sequence or a ribosomal skip sequence, e.g., T2A. See WO2014031687. In some embodiments, introduction of a construct encoding the CAR and EGFRt separated by a T2A ribosome switch can express two proteins from the same construct, such that the EGFRt can be used as a marker to detect cells expressing such construct. In some embodiments, a marker, and optionally a linker sequence, can be any as disclosed in published patent application No. WO2014031687. For example, the marker can be a truncated EGFR (tEGFR) that is, optionally, linked to a linker sequence, such as a T2A ribosomal skip sequence.

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

The TCR or CAR may comprise one or modified synthetic amino acids in place of one or more naturally-occurring amino acids. Exemplary modified amino acids include, but are not limited to, aminocyclohexane carboxylic acid, norleucine, α-amino n-decanoic acid, homoserine, S-acetylaminomethylcysteine, trans-3- and trans-4-hydroxyproline, 4-aminophenylalanine, 4- nitrophenylalanine, 4-chlorophenylalanine, 4-carboxyphenylalanine, (3-phenylserine (3-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N’-methyl-lysine, N′,N′-dibenzyl-lysine, 6- hydroxylysine, omithine, α-aminocyclopentane carboxylic acid, α-aminocyclohexane carboxylic acid, α-aminocycloheptane carboxylic acid, α-(2-amino-2-norbomane)-carboxylic acid, α,γ-diaminobutyric acid, α,γ-diaminopropionic acid, homophenylalanine, and α-tertbutylglycine.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR in some aspects is one that includes multiple costimulatory domains of different costimulatory receptors.

In some embodiments, the chimeric antigen receptor includes an extracellular portion containing a TCR or fragment described herein. In some aspects, the chimeric antigen receptor includes an extracellular portion containing a TCR or fragment described herein and an intracellular signaling domain. In some embodiments, the intracellular domain contains an ITAM. In some aspects, the intracellular signaling domain includes a signaling domain of a zeta chain of a CD3-zeta (CD3) chain. In some embodiments, the chimeric antigen receptor includes a transmembrane domain linking the extracellular domain and the intracellular signaling domain.

In some aspects, the transmembrane domain contains a transmembrane portion of CD28. The extracellular domain and transmembrane can be linked directly or indirectly. In some embodiments, the extracellular domain and transmembrane are linked by a spacer, such as any described herein. In some embodiments, the chimeric antigen receptor contains an intracellular domain of a T cell costimulatory molecule, such as between the transmembrane domain and intracellular signaling domain. In some aspects, the T cell costimulatory molecule is CD28 or 41BB.

In some embodiments, the CAR contains a TCR, e.g., a TCR fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains a TCR, e.g., a TCR fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the receptor, e.g., the TCR or CAR, is a transmembrane domain of human CD28 or variant thereof, e.g., a 27-amino acid transmembrane domain of a human CD28 (Accession No.: P10747.1).

In some embodiments, the chimeric antigen receptor contains an intracellular domain of a T cell costimulatory molecule. In some aspects, the T cell costimulatory molecule is CD28 or 41BB.

In some embodiments, the intracellular signaling domain comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular domain comprises an intracellular costimulatory signaling domain of 41BB or functional variant or portion thereof, such as a 42-amino acid cytoplasmic domain of a human 4-1BB (Accession No. Q07011.1) or functional variant or portion thereof.

In some embodiments, the intracellular signaling domain comprises a human CD3 zeta stimulatory signaling domain or functional variant thereof, such as a 112 AA cytoplasmic domain of isoform 3 of human CD3.zeta. (Accession No.: P20963.2) or a CD3 zeta signaling domain as described in U.S. Pat. No. 7,446,190 or 8,911,993.

In some aspects, the spacer contains only a hinge region of an IgG, such as only a hinge of IgG4 or IgG1. In other embodiments, the spacer is an Ig hinge, e.g., and IgG4 hinge, linked to a CH2 and/or CH3 domains. In some embodiments, the spacer is an Ig hinge, e.g., an IgG4 hinge, linked to CH2 and CH3 domains. In some embodiments, the spacer is an Ig hinge, e.g., an IgG4 hinge, linked to a CH3 domain only. In some embodiments, the spacer is or comprises a glycine-serine rich sequence or other flexible linker such as known flexible linkers.

For example, in some embodiments, the CAR includes a TCR or fragment thereof, such as any of the HLA-PEPTIDE specific TCRs, a spacer such as any of the Ig-hinge containing spacers, a CD28 transmembrane domain, a CD28 intracellular signaling domain, and a CD3 zeta signaling domain. In some embodiments, the CAR includes a TCR or fragment, such as any of the HLA-PEPTIDE specific TCRs, a spacer such as any of the Ig-hinge containing spacers, a CD28 transmembrane domain, a CD28 intracellular signaling domain, and a CD3 zeta signaling domain.

Nucleotides, Vectors, Host Cells, and Related Methods

Also provided are isolated nucleic acids encoding the ABPs or antigens disclosed herein, vectors comprising the nucleic acids, and host cells comprising the vectors and nucleic acids, as well as recombinant techniques for the production of the ABPs.

The nucleic acids may be recombinant. The recombinant nucleic acids may be constructed outside living cells by joining natural or synthetic nucleic acid segments to nucleic acid molecules that can replicate in a living cell, or replication products thereof. For purposes herein, the replication can be in vitro replication or in vivo replication.

For recombinant production of an ABP, the nucleic acid(s) encoding it may be isolated and inserted into a replicable vector for further cloning (i.e., amplification of the DNA) or expression. In some aspects, the nucleic acid may be produced by homologous recombination, for example as described in U.S. Pat. No. 5,204,244, incorporated by reference in its entirety.

Many different vectors are known in the art. The vector components generally include one or more of the following: a signal sequence, an origin of replication, one or more marker genes, an enhancer element, a promoter, and a transcription termination sequence, for example as described in U.S. Pat. No. 5,534,615, incorporated by reference in its entirety.

Exemplary vectors or constructs suitable for expressing an ABP, e.g., a CAR, or antigen binding fragment thereof, include, e.g., the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as AGT1O, AGT11, AZapII (Stratagene), AEMBL4, and ANMI 149, are also suitable for expressing an ABP disclosed herein.

Illustrative examples of suitable host cells are provided below. These host cells are not meant to be limiting, and any suitable host cell may be used to produce the ABPs provided herein.

Suitable host cells include any prokaryotic (e.g., bacterial), lower eukaryotic (e.g., yeast), or higher eukaryotic (e.g., mammalian) cells. Suitable prokaryotes include eubacteria, such as Gram-negative or Gram-positive organisms, for example, Enterobacteriaceae such as Escherichia (E. coli), Enterobacter, Erwinia, Klebsiella, Proteus, Salmonella (S. typhimurium), Serratia (S. marcescans), Shigella, Bacilli (B. subtilis and B. licheniformis), Pseudomonas (P. aeruginosa), and Streptomyces. One useful E. coli cloning host is E. coli 294, although other strains such as E. coli B, E. coli X1776, and E. coli W3110 are also suitable.

In addition to prokaryotes, eukaryotic microbes such as filamentous fungi or yeast are also suitable cloning or expression hosts for ABP-encoding vectors. Saccharomyces cerevisiae, or common baker's yeast, is a commonly used lower eukaryotic host microorganism. However, a number of other genera, species, and strains are available and useful, such as Schizosaccharomyces pombe, Kluyveromyces (K lactis, K. fragilis, K. bulgaricus K. wickeramii, K. waltii, K. drosophilarum, K. thermotolerans, and K. marxianus), Yarrowia, Pichia pastoris, Candida (C. albicans), Trichoderma reesia, Neurospora crassa, Schwanniomyces (S. occidentalis), and filamentous fungi such as, for example Penicillium, Tolypocladium, and Aspergillus (A. nidulans and A. niger).

Useful mammalian host cells include COS-7 cells, HEK293 cells; baby hamster kidney (BHK) cells; Chinese hamster ovary (CHO); mouse sertoli cells; African green monkey kidney cells (VERO-76), and the like.

The host cells used to produce the HLA-PEPTIDE ABP may be cultured in a variety of media. Commercially available media such as, for example, Ham's F10, Minimal Essential Medium (MEM), RPMI-1640, and Dulbecco's Modified Eagle's Medium (DMEM) are suitable for culturing the host cells. In addition, any of the media described in Ham et al., Meth. Enz., 1979, 58:44; Barnes et al., Anal. Biochem., 1980, 102:255; and U.S. Pat. Nos. 4,767,704, 4,657,866, 4,927,762, 4,560,655, and 5,122,469; or WO 90/03430 and WO 87/00195 may be used. Each of the foregoing references is incorporated by reference in its entirety.

Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics, trace elements (defined as inorganic compounds usually present at final concentrations in the micromolar range), and glucose or an equivalent energy source. Any other necessary supplements may also be included at appropriate concentrations that would be known to those skilled in the art.

The culture conditions, such as temperature, pH, and the like, are those previously used with the host cell selected for expression, and will be apparent to the ordinarily skilled artisan.

When using recombinant techniques, the ABP can be produced intracellularly, in the periplasmic space, or directly secreted into the medium. If the ABP is produced intracellularly, as a first step, the particulate debris, either host cells or lysed fragments, is removed, for example, by centrifugation or ultrafiltration. For example, Carter et al. (Bio Technology, 1992, 10:163-167, incorporated by reference in its entirety) describes a procedure for isolating ABPs which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation.

In some embodiments, the ABP is produced in a cell-free system. In some aspects, the cell-free system is an in vitro transcription and translation system as described in Yin et al., mAbs, 2012, 4:217-225, incorporated by reference in its entirety. In some aspects, the cell-free system utilizes a cell-free extract from a eukaryotic cell or from a prokaryotic cell. In some aspects, the prokaryotic cell is E. coli. Cell-free expression of the ABP may be useful, for example, where the ABP accumulates in a cell as an insoluble aggregate, or where yields from periplasmic expression are low.

Where the ABP is secreted into the medium, supernatants from such expression systems are generally first concentrated using a commercially available protein concentration filter, for example, an Amicon® or Millipore® Pellcon® ultrafiltration unit. A protease inhibitor such as PMSF may be included in any of the foregoing steps to inhibit proteolysis and antibiotics may be included to prevent the growth of adventitious contaminants.

The ABP composition prepared from the cells can be purified using, for example, hydroxylapatite chromatography, gel electrophoresis, dialysis, and affinity chromatography, with affinity chromatography being a particularly useful purification technique. The suitability of protein A as an affinity ligand depends on the species and isotype of any immunoglobulin Fc domain that is present in the ABP. Protein A can be used to purify ABPs that comprise human γ1, γ2, or γ4 heavy chains (Lindmark et al., J. Immunol. Meth., 1983, 62:1-13, incorporated by reference in its entirety). Protein G is useful for all mouse isotypes and for human γ3 (Guss et al., EMBO J., 1986, 5:1567-1575, incorporated by reference in its entirety).

The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the ABP comprises a CH3 domain, the BakerBond ABX® resin is useful for purification.

Other techniques for protein purification, such as fractionation on an ion-exchange column, ethanol precipitation, Reverse Phase HPLC, chromatography on silica, chromatography on heparin Sepharose*, chromatofocusing, SDS-PAGE, and ammonium sulfate precipitation are also available, and can be applied by one of skill in the art.

Following any preliminary purification step(s), the mixture comprising the ABP of interest and contaminants may be subjected to low pH hydrophobic interaction chromatography using an elution buffer at a pH between about 2.5 to about 4.5, generally performed at low salt concentrations (e.g., from about 0 to about 0.25 M salt).

Methods of Making HLA-PEPTIDE ABPs

### HLA-PEPTIDE Antigen Preparation

The HLA-PEPTIDE antigen used for isolation or creation of the ABPs provided herein may be intact HLA-PEPTIDE or a fragment of HLA-PEPTIDE. The HLA-PEPTIDE antigen may be, for example, in the form of isolated protein or a protein expressed on the surface of a cell.

In some embodiments, the HLA-PEPTIDE antigen is anon-naturally occurring variant of HLA-PEPTIDE, such as a HLA-PEPTIDE protein having an amino acid sequence or post-translational modification that does not occur in nature.

In some embodiments, the HLA-PEPTIDE antigen is truncated by removal of, for example, intracellular or membrane-spanning sequences, or signal sequences. In some embodiments, the HLA-PEPTIDE antigen is fused at its C-terminus to a human IgG1 Fc domain or a polyhistidine tag.

Methods and Systems for Identifying ABPs

ABPs that bind HLA-PEPTIDE can be identified using any method known in the art, e.g., phage display, immunization of a subject, or isolation of an ABP expressing cell and subsequent sequencing of the ABP.

One method of identifying an antigen binding protein includes providing at least one HLA-PEPTIDE target; and binding the at least one target with an antigen binding protein, thereby identifying the antigen binding protein. The antigen binding protein can be present in a library comprising a plurality of distinct antigen binding proteins.

In some embodiments, the library is a phage display library. The phage display library can be developed so that it is substantially free of antigen binding proteins that non-specifically bind the HLA of the HLA-PEPTIDE target. The antigen binding protein can be present in a yeast display library comprising a plurality of distinct antigen binding proteins. The yeast display library can be developed so that it is substantially free of antigen binding proteins that non-specifically bind the HLA of the HLA-PEPTIDE target.

In some embodiments, the library is a yeast display library.

In some aspects, the binding step is performed more than once, optionally at least three times, e.g., at least 1, 2, 3, 4, 5, 6, 7, 8, 9, or 10×.

In addition, the method can also include contacting the antigen binding protein with one or more peptide-HLA complexes that are distinct from the HLA-PEPTIDE target to determine if the antigen binding protein selectively binds the HLA-PEPTIDE target.

Accordingly, provided herein are systems for identifying an ABP that selectively binds one or more antigens described herein. In some embodiments, the system comprises (a) an isolated antigen comprising an HLA-restricted peptide complexed with an HLA Class I molecule, wherein the HLA-restricted peptide is located in the peptide binding groove of an α1/α2 heterodimer portion of the HLA Class I molecule, and wherein the antigen is selected from an antigen described in any one of SEQ ID NOs:10,755 to 29,364; and (b) a library comprising a plurality of distinct antigen binding proteins. In some embodiments, the library is a phage display library.

In some embodiments of the system, the antigen is attached to a solid support. The solid support can comprise, e.g., a bead, well, membrane, tube, column, plate, sepharose, magnetic bead, cell, or chip. In some embodiments, the antigen comprises a first member of an affinity binding pair and the solid support comprises a second member of the affinity binding pair. In some embodiments, the first member is streptavidin and the second member is biotin. In some embodiments, the antigen attached to a solid support is an HLA-multimer (e.g., a tetramer) comprising at least one HLA-PEPTIDE target.

In some embodiments of the system, the library (e.g., the phage display library) is a human library. In some embodiments of the system, the library (e.g., the phage display library) is a humanized library.

In some embodiments, the system further comprises a negative control antigen comprising an HLA-restricted peptide complexed with an HLA Class I molecule, wherein the HLA-restricted peptide is located in the peptide binding groove of an α1/α2 heterodimer portion of the HLA Class I molecule, and wherein the negative control antigen comprises a different restricted peptide, a different HLA Class I molecule, or a different restricted peptide and a different HLA Class I molecule. In some embodiments, the negative control antigen comprises a different restricted peptide but the same HLA Class I molecule as the antigen.

In some embodiments, the system comprises a reaction mixture, the reaction mixture comprising the antigen and a plurality of phages from the phage display library.

Another method of identifying an antigen binding protein can include obtaining at least one HLA-PEPTIDE target; administering the HLA-PEPTIDE target to a subject (e.g., a mouse, rabbit or a llama), optionally in combination with an adjuvant; and isolating the antigen binding protein from the subject. Isolating the antigen binding protein can include screening the serum of the subject to identify the antigen binding protein. The method can also include contacting the antigen binding protein with one or more peptide-HLA complexes that are distinct from the HLA-PEPTIDE target, e.g., to determine if the antigen binding protein selectively binds to the HLA-PEPTIDE target. An antigen binding protein that is identified can be humanized.

In some aspects, isolating the antigen binding protein comprises isolating a T cell from the subject that expresses the antigen binding protein. The T cell can be used to create a hybridoma. The T cell can also be used for cloning one or more of its CDRs. The T cell can also be immortalized, for example, by using EBV transformation. Sequences encoding an antigen binding protein can be cloned from immortalized T cells or can be cloned directly from T cells isolated from an immunized subject. A library that comprises the antigen binding protein of the T cell can also be created, optionally wherein the library is phage display or yeast display.

Another method of identifying an antigen binding protein can include obtaining a cell comprising the antigen binding protein (ABP); contacting the cell with an HLA-multimer (e.g., a tetramer) comprising at least one HLA-PEPTIDE target; and identifying the antigen binding protein via binding between the HLA-multimer and the antigen binding protein.

Another method of identifying an antigen binding protein can include obtaining a cell comprising the antigen binding protein (ABP) and determining the sequence of the ABP. For example, the method can include contacting the cell with an HLA-multimer (e.g., a tetramer) comprising at least one HLA-PEPTIDE target; isolating the cell, optionally using flow cytometry (e.g. fluorescent activated cell sorting “FACS”), magnetic separation, or single cell separation; and sequencing polynucleotides from the isolated cell to determine the sequence of the ABP.

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (marker^{high}) on the positively or negatively selected cells, respectively. For example, a population of cells known or suspected to contain T cells can be positively sorted based on binding to a tetramer containing a HLA-PEPTIDE of interest (e.g., a neoantigen). FACS isolation can also include removing cells that bind to a HLA-PEPTIDE target that is not of interest. For example, cells can be positively sorted based on binding to a tetramer containing a HLA-PEPTIDE of interest (e.g., a neoantigen) and negatively sorted based on binding to a tetramer containing a HLA-PEPTIDE not of interest (e.g., the wildtype peptide sequence corresponding to a neoantigen of interest).

Isolation of cells expressing an ABP-containing protein (e.g, FACS-based isolation of T cells), can include isolation of subject-derived cells. Subject-derived cells can be isolated from a variety of biological samples including, but not limited to, body fluids, such as blood, plasma, serum, cerebrospinal fluid, synovial fluid, urine and sweat, tissue and organ samples. The biological sample can be a sample obtained directly from a biological source or a sample that is processed. The sample from which the subject-derived cells are derived or isolated can be a blood or a blood-derived sample, or can be derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Exemplary cells and cell populations expressing an ABP-containing protein include, but are not limited to, an activated T cell, a tumor infiltrating lymphocyte (TIL), a PBMC, a cultured (e.g., expanded) T cell, a naive T (TN) cell, an effector T cell (TEFF), a memory T cell, a stem cell memory T cell (TSCM), a central memory T cell (TCM), an effector memory T cell (TEM), a terminally differentiated effector memory T cell, an immature T cell, a mature T cell, a helper T cell, a cytotoxic T cell, a mucosa-associated invariant T (MALT) cell, a regulatory T cell (Treg), a TH1 cell, a TH2 cell, a TH3 cell, a TH17 cell, a TH9 cell, a TH22 cell, a follicular helper T cell, an natural killer T cell (NKT), an alpha-beta T cell, and a gamma-delta T cell.

Sequencing of cells expressing an ABP-containing protein can carried out by techniques known to those skilled in the art, such as the Chromium Single Cell Immune Profiling system (10× Genomics).

Another method of identifying an antigen binding protein can include obtaining one or more cells comprising the antigen binding protein; activating the one or more cells with at least one HLA-PEPTIDE target presented on at least one antigen presenting cell (APC); and identifying the antigen binding protein via selection of one or more cells activated by interaction with at least one HLA-PEPTIDE target.

The cell can be, e.g., a T cell, optionally a CTL, or an NK cell, for example. The method can further include isolating the cell, optionally using flow cytometry, magnetic separation, or single cell separation. The method can further include sequencing the antigen binding protein.

Methods for Engineering Cells with ABPs

Also provided are methods, nucleic acids, compositions, and kits, for expressing the ABPs, including receptors comprising TCRs, CARs, and the like, and for producing genetically engineered cells expressing such ABPs. The genetic engineering generally involves introduction of a nucleic acid encoding the recombinant or engineered component into the cell, such as by retroviral transduction, transfection, or transformation.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some contexts, overexpression of a stimulatory factor (for example, a lymphokine or a cytokine) may be toxic to a subject. Thus, in some contexts, the engineered cells include segments that cause the cells to be susceptible to negative selection in vivo, such as upon administration in adoptive immunotherapy. For example in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell II: 223, 1977) which confers ganciclovir sensitivity; the cellular hypoxanthine phosphribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

In some aspects, the cells are further engineered to promote expression of cytokines or other factors. Various methods for the introduction of genetically engineered components, such as antigen receptors (e.g., TCRs), are well known and may be used with the provided methods and compositions. Exemplary methods include those for transfer of nucleic acids encoding the receptors, including via viral, e.g., retroviral or lentiviral transduction, transposons, nuclease mediated gene-editing (e.g., CRISPR, TALEN, meganuclease, or ZFN editing systems), and electroporation. For example, nuclease mediated gene-editing, particularly for editing T cells, is described in more detail in international applications WO/2018/232356 and PCT/US2018/058230, herein incorporated by reference for all purposes.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt. 2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation (see, e.g., Chicaybam et al, (2013) PLoS ONE 8(3): e60298; Van Tedeloo et al. (2000) Gene Therapy 7(16): 1431-1437; and Roth et al. (2018) Nature 559:405-409). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

Preparation of Engineered Cells

In some embodiments, preparation of the engineered cells includes one or more culture and/or preparation steps. The cells for introduction of the HLA-PEPTIDE-ABP, e.g., TCRs, can be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, or pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca++/Mg++ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques.

For example, CD3+, CD28+ T cells can be positively selected using CD3/CD28 conjugated magnetic beads (e.g., DYNABEADS.® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (marker^{high}) on the positively or negatively selected cells, respectively.

In some embodiments, T cells are separated from a peripheral blood mononuclear cell (PBMC) sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations.

In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. See Terakura et al. (2012) Blood. 1:72-82; Wang et al. (2012) J Immunother. 35(9):689-701. In some embodiments, combining TCM-enriched CD8+ T cells and CD4+ T cells further enhances efficacy.

In embodiments, memory T cells are present in both CD62L+ and CD62L-subsets of CD8+ peripheral blood lymphocytes. Peripheral blood mononuclear cell (PBMC) can be enriched for or depleted of CD62L-CD8+ and/or CD62L+CD8+ fractions, such as using anti-CD8 and anti-CD62L antibodies.

In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4+ cells, where both the negative and positive fractions are retained. The negative fraction then is subjected to negative selection based on expression of CD14 and CD45RA or ROR1, and positive selection based on a marker characteristic of central memory T cells, such as CD62L or CCR7, where the positive and negative selections are carried out in either order. 100,1339 CD4+T helper cells are sorted into naive, central memory, and effector cells by identifying cell populations that have cell surface antigens. CD4+ lymphocytes can be obtained by standard methods. In some embodiments, naive CD4+T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L- and CD45RO−.

In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immune-magnetic (or affinity-magnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynabeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the affinity-based selection is via magnetic-activated cell sorting (MACS) (Miltenyi Biotech, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labeled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood may be automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood. 1:72-82, and Wang et al. (2012) J Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale fluorescence activated cell sorting (FACS). In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This can then be diluted 1:1 with media so that the final concentration of DMSO and HSA are 10% and 4%, respectively. Other examples include Cryostor®, CTL-Cryo™ ABC freezing media, and the like. The cells are then frozen to −80 degrees C. at a rate of 1 degree per minute and stored in the vapor phase of a liquid nitrogen storage tank.

In some embodiments, the provided methods include cultivation, incubation, culture, and/or genetic engineering steps. For example, in some embodiments, provided are methods for incubating and/or engineering the depleted cell populations and culture-initiating compositions.

Thus, in some embodiments, the cell populations are incubated in a culture-initiating composition. The incubation and/or engineering may be carried out in a culture vessel, such as a unit, chamber, well, column, tube, tubing set, valve, vial, culture dish, bag, or other container for culture or cultivating cells.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some embodiments, the PBMC feeder cells are inactivated with Mytomicin C. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

Assays

A variety of assays known in the art may be used to identify and characterize an HLA-PEPTIDE ABP provided herein.

### Binding, Competition, and Epitope Mapping Assays

Specific antigen-binding activity of an ABP provided herein may be evaluated by any suitable method, including using SPR, BLI, RIA, Carterra biosensor, and MSD-SET, as described elsewhere in this disclosure. Additionally, antigen-binding activity may be evaluated by ELISA assays, using flow cytometry, and/or Western blot assays.

Assays for measuring competition between two ABPs, or an ABP and another molecule (e.g., one or more ligands of HLA-PEPTIDE such as a TCR) are described elsewhere in this disclosure and, for example, in Harlow and Lane, ABPs: A Laboratory Manual ch. 14, 1988, Cold Spring Harbor Laboratory, Cold Spring Harbor, N.Y, incorporated by reference in its entirety.

Assays for mapping the epitopes to which an ABP provided herein bind are described, for example, in Morris “Epitope Mapping Protocols,” in Methods in Molecular Biology vol. 66, 1996, Humana Press, Totowa, N.J., incorporated by reference in its entirety.

In some embodiments, the epitope is determined by peptide competition. In some embodiments, the epitope is determined by mass spectrometry. In some embodiments, the epitope is determined by mutagenesis. In some embodiments, the epitope is determined by crystallography.

### Assays for Effector Functions

Effector function following treatment with an ABP and/or cell provided herein may be evaluated using a variety of in vitro and in vivo assays known in the art, including those described in Ravetch and Kinet, Annu. Rev. Immunol., 1991, 9:457-492; U.S. Pat. Nos. 5,500,362, 5,821,337; Hellstrom et al., Proc. Nat'l Acad. Sci. USA, 1986, 83:7059-7063; Hellstrom et al., Proc. Nat'l Acad. Sci. USA, 1985, 82:1499-1502; Bruggemann et al., J. Exp. Med., 1987, 166:1351-1361; Clynes et al., Proc. Nat'l Acad. Sci. USA, 1998, 95:652-656; WO 2006/029879; WO 2005/100402; Gazzano-Santoro et al., J. Immunol. Methods, 1996, 202:163-171; Cragg et al., Blood, 2003, 101:1045-1052; Cragg et al. Blood, 2004, 103:2738-2743; and Petkova et al., Int'l. Immunol., 2006, 18:1759-1769; each of which is incorporated by reference in its entirety.

Pharmaceutical Compositions

An ABP, cell, or HLA-PEPTIDE target provided herein can be formulated in any appropriate pharmaceutical composition and administered by any suitable route of administration. Suitable routes of administration include, but are not limited to, the intra-arterial, intradermal, intramuscular, intraperitoneal, intravenous, nasal, parenteral, pulmonary, and subcutaneous routes.

The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Sheskey et al. (Eds.) 8th Ed. (2017), incorporated by reference in its entirety.

In some embodiments, the pharmaceutical composition comprises a carrier.

Methods of Treatment

For therapeutic applications, ABPs and/or cells are administered to a mammal, generally a human, in a pharmaceutically acceptable dosage form such as those known in the art and those discussed above. For example, ABPs and/or cells may be administered to a human intravenously as a bolus or by continuous infusion over a period of time, by intramuscular, intraperitoneal, intra-cerebrospinal, subcutaneous, intra-articular, intrasynovial, intrathecal, or intratumoral routes. The ABPs also are suitably administered by peritumoral, intralesional, or perilesional routes, to exert local as well as systemic therapeutic effects. The intraperitoneal route may be particularly useful, for example, in the treatment of ovarian tumors.

The ABPs and/or cells provided herein can be useful for the treatment of any disease or condition involving HLA-PEPTIDE antigen. In some embodiments, the disease or condition is a disease or condition that can benefit from treatment with an anti-HLA-PEPTIDE ABP and/or cell. In some embodiments, the disease or condition is a tumor. In some embodiments, the disease or condition is a cell proliferative disorder. In some embodiments, the disease or condition is a cancer.

In some embodiments, the ABPs and/or cells provided herein are provided for use as a medicament. In some embodiments, the ABPs and/or cells provided herein are provided for use in the manufacture or preparation of a medicament. In some embodiments, the medicament is for the treatment of a disease or condition that can benefit from an anti-HLA-PEPTIDE ABP and/or cell. In some embodiments, the disease or condition is a tumor. In some embodiments, the disease or condition is a cell proliferative disorder. In some embodiments, the disease or condition is a cancer.

Provided herein is a method of treating a disease or condition in a subject in need thereof by administering an effective amount of an ABP and/or cell provided herein to the subject. In some aspects, the disease or condition is a cancer.

Also provided herein is a method of treating a disease or condition in a subject in need thereof by administering an effective amount of an ABP and/or cell provided herein to the subject, wherein the disease or condition is a cancer, and the cancer is selected from a solid tumor and a hematological tumor.

Also provided herein is a method of modulating an immune response in a subject in need thereof, comprising administering to the subject an effective amount of an ABP and/or cell or a pharmaceutical composition disclosed herein.

A modulated immune response in the subject may be evaluated by any means known in the art.

In some embodiments, a modulated immune response in the subject comprises an increase in or induction of antibody-dependent cellular toxicity (ADCC), e.g., of a target cell with surface expression of the neoantigen target of the ABP. ADCC can be evaluated by any means known in the art.

In some embodiments, a modulated immune response in the subject comprises an increase in or induction of complement dependent cytotoxicity (CDC), e.g., of a target cell with surface expression of the neoantigen target of the ABP. CDC can be evaluated by any means known in the art.

By way of example only, immune response in the subject can be evaluated by evaluating lymphocytes obtained from the subject or the subject's tumor for binding to the HLA-PEPTIDE antigen. In some embodiments, tumor-infiltrating lymphocytes from the subject or evaluated for binding to the HLA-PEPTIDE antigen. By way of other example only, modulated immune response in the subject can include an expansion of pre-existing neoantigen-specific T cell population, a broader repertoire of new T-cell specificities in the subject, or both. Methods for evaluating such modulated immune response are described in Ott et al., An immunogenic personal neoantigen vaccine for patients with melanoma. Nature 547, 217-221 (13 Jul. 2017), which is hereby incorporated be reference in its entirety. Methods for evaluating immune response are also described in Sahin et al., Personalized RNA mutanome vaccines mobilize poly-specific therapeutic immunity against cancer, Nature 547, 222-226 (13 Jul. 2017), which is hereby incorporated by reference in its entirety.

To perform immune monitoring, PBMCs are commonly used. PBMCs can be isolated before prime vaccination, and after prime vaccination (e.g. 4 weeks and 8 weeks). PBMCs can be harvested just prior to boost vaccinations and after each boost vaccination (e.g. 4 weeks and 8 weeks).

In some embodiments, a modulated immune response in the subject comprises a modulated T cell response. T cell responses can be measured using one or more methods known in the art such as ELISpot, intracellular cytokine staining, cytokine secretion and cell surface capture, T cell proliferation, MHC multimer staining, or by cytotoxicity assay. T cell responses to HLA-PEPTIDE antigens disclosed herein can be monitored from PBMCs by measuring induction of cytokines, such as IFN-gamma, using an ELISpot assay. Specific CD4 or CD8 T cell responses to HLA-PEPTIDE antigens disclosed herein can be monitored from PBMCs by measuring induction of cytokines captured intracellularly or extracellularly, such as IFN-gamma, using flow cytometry. Specific CD4 or CD8 T cell responses to HLA-PEPTIDE antigens disclosed herein can be monitored from PBMCs by measuring T cell populations expressing T cell receptors specific for epitope/MHC class I complexes using MHC multimer staining. Specific CD4 or CD8 T cell responses to HLA-PEPTIDE antigens disclosed herein can be monitored from PBMCs by measuring the ex vivo expansion of T cell populations following 3H-thymidine, bromodeoxyuridine and carboxyfluoresceine-diacetate-succinimidylester (CFSE) incorporation. The antigen recognition capacity and lytic activity of PBMC-derived T cells that are specific HLA-PEPTIDE antigens disclosed herein can be assessed functionally by chromium release assay or alternative colorimetric cytotoxicity assays.

In particular embodiments, immune response in the subject is evaluated by enzyme linked immunospot (ELISPOT) analysis.

Also provided herein is a method of killing a target cell in a subject in need thereof, comprising administering to the subject an effective amount of an ABP and/or cell or a pharmaceutical composition disclosed herein. In some embodiments, the subject has cancer.

In some embodiments, the target cell is a cancer cell.

In some embodiments, the cancer or cancer cell expresses or is predicted to express an HLA-PEPTIDE antigen or HLA Class I molecule as described in any one of SEQ ID NOs:10,755 to 29,364. In some embodiments, the cancer or cancer cell is determined or predicted to comprise the somatic mutation in the gene that is associated with the HLA-PEPTIDE antigen. In some embodiments, the ABP selectively binds to the HLA-PEPTIDE antigen. In some embodiments, the ABP selectively binds to the HLA Class I subtype comprised in the HLA-PEPTIDE antigen.

In some embodiments, prior to administering the ABP to the subject, the cancer or cancer cell of the subject, or a biological sample from the subject, is determined to express the HLA-PEPTIDE antigen. In some embodiments, prior to administering the ABP to the subject, the cancer or cancer cell of the subject, or a biological sample from the subject, is determined to comprise the HLA Class I subtype of the HLA-PEPTIDE antigen. By way of example only, prior to administering an ABP that selectively binds to RAS G12D neoantigen HLA-A*11:01_VVVGADGVGK (“SNA30”), the cancer or cancer cell of the subject, or a biological sample from the subject, is determined to comprise HLA-A*11:01. In some embodiments, prior to administering the ABP to the subject, the cancer or cancer cell of the subject, or a biological sample from the subject, is determined to comprise the somatic mutation in the gene that is associated with the HLA-PEPTIDE antigen. By way of example only, prior to administering an ABP that selectively binds to RAS G12D neoantigen HLA-A*11:01_VVVGADGVGK (“SNA30”), the cancer or cancer cell of the subject, or a biological sample from the subject, is determined to comprise a RAS G12D mutation. In some embodiments, prior to administering the ABP to the subject, the cancer or cancer cell of the subject, or a biological sample from the subject, is determined to comprise the HLA Class I subtype of the HLA-PEPTIDE antigen and the cancer or cancer cell of the subject expresses or is predicted to express the gene associated with the somatic alteration encompassed by the HLA-PEPTIDE antigen. By way of example only, prior to administering an ABP that selectively binds to RAS G12D neoantigen HLA-A*11:01_VVVGADGVGK (“SNA30”), the cancer or cancer cell of the subject, or a biological sample from the subject is determined to comprise HLA-A*11:01 and the cancer or cancer cell of the subject expresses RAS, e.g., KRAS, NRAS, or HRAS. In some embodiments, prior to administering the ABP to the subject, the cancer, cancer cell, or biological sample of the subject is determined to comprise the somatic mutation in the gene that is associated with the HLA-PEPTIDE antigen, and the subject is determined to express the HLA Class I subtype comprised in the HLA-PEPTIDE antigen. By way of example only, prior to administering an ABP that selectively binds to RAS G12D neoantigen HLA-A*11:01_VVVGADGVGK (“SNA30”), the cancer or cancer cell of the subject, or a biological sample from the subject is determined to comprise HLA-A*11:01 and a RAS G12D mutation. In some embodiments, a biological sample obtained from the subject is determined to be positive for the HLA-PEPTIDE antigen or HLA Class I subtype comprised in the HLA-PEPTIDE antigen. In some embodiments, a cancer or cancer cell of the subject is determined to express the gene associated with the somatic alteration, the mutation, or both the gene and the somatic alteration, above a predefined threshold. In some embodiments, loss of the HLA Class I subtype in the cancer or cancer cell of the subject is not detected.

Biological samples include, but are not limited to, body fluids, such as blood, plasma, serum, cerebrospinal fluid, synovial fluid, urine and sweat, tissue and organ samples, including processed samples derived therefrom. In some embodiments, the biological sample comprises a tumor sample, e.g., a solid tumor sample. In some embodiments, the solid tumor sample is a fresh tumor sample. In some embodiments, the solid tumor sample is a frozen tumor sample. In some embodiments, the tumor sample is a formalin-fixed, paraffin-embedded (FFPE) sample. In some embodiments, the tumor sample is a tumor biopsy or resection preserved in an agent formulated to prevent RNA degradation in the sample. Such agents are known in the art, and include, but are not limited to, RNAlater. In some embodiments, the biological sample is a liquid sample. In particular embodiments, the liquid sample is a blood sample. In particular embodiments, the blood sample is a whole blood sample. In particular embodiments, the blood sample is a plasma sample. In particular embodiments, the blood sample is a serum sample.

By way of example only, if a cancer, cancer cell, or biological sample of the subject is determined to comprise a CREB3L1 V414I mutation, the subject may be selected for treatment with an ABP that selectively binds the HLA-PEPTIDE neoantigen designated as SEQ ID NO: 10755. By way of other example only, if the subject is determined to express the HLA Class I allele HLA-A*02:06, the subject may be selected for treatment with an ABP that selectively binds the HLA-PEPTIDE neoantigen designated as SEQ ID NO: 10755. By way of yet other example only, if the subject is determined to express the HLA Class I allele HLA-A*02:06 and a cancer, cancer cell, or biological sample of the subject is determined to comprise a CREB3L1 V414I mutation, the subject may be selected for treatment with an ABP that selectively binds the HLA-PEPTIDE neoantigen designated as SEQ ID NO: 10755.

By way of example only, if a cancer, cancer cell, or biological sample of the subject is determined to comprise a RAS G12C mutation, the subject may be selected for treatment with an ABP that selectively binds the HLA-PEPTIDE neoantigen designated as SEQ ID NO: 14954 and 14955. By way of other example only, if the subject is determined to express the HLA Class I allele HLA-A*02:01, the subject may be selected for treatment with an ABP that selectively binds the HLA-PEPTIDE neoantigen designated as SEQ ID NO: 14954 and 14955. By way of yet other example only, if the subject is determined to express the HLA Class I allele HLA-A*02:01 and a cancer, cancer cell, or biological sample of the subject is determined to comprise a RAS G12C mutation, the subject may be selected for treatment with an ABP that selectively binds the HLA-PEPTIDE neoantigen designated as SEQ ID NO: 14954 and 14955.

By way of example only, if a cancer, cancer cell, or biological sample of the subject is determined to comprise a RAS G12D mutation, the subject may be selected for treatment with an ABP that selectively binds the HLA-PEPTIDE neoantigen designated as SEQ ID NO: 19865. By way of other example only, if the subject is determined to express the HLA Class I allele HLA-A*11:01, the subject may be selected for treatment with an ABP that selectively binds the HLA-PEPTIDE neoantigen designated as SEQ ID NO: 19865. By way of yet other example only, if the subject is determined to express the HLA Class I allele HLA-A*11:01 and a cancer, cancer cell, or biological sample of the subject is determined to comprise a RAS G12D mutation, the subject may be selected for treatment with an ABP that selectively binds the HLA-PEPTIDE neoantigen designated as SEQ ID NO: 19865.

By way of example only, if a cancer, cancer cell, or biological sample of the subject is determined to comprise a RAS G12V mutation, the subject may be selected for treatment with an ABP that selectively binds the HLA-PEPTIDE neoantigen designated as SEQ ID NO: 19,976. By way of other example only, if the subject is determined to express the HLA Class I allele HLA-A*11:01, the subject may be selected for treatment with an ABP that selectively binds the HLA-PEPTIDE neoantigen designated as SEQ ID NO: 19,976. By way of yet other example only, if the subject is determined to express the HLA Class I allele HLA-A*11:01 and a cancer, cancer cell, or biological sample of the subject is determined to comprise a RAS G12V mutation, the subject may be selected for treatment with an ABP that selectively binds the HLA-PEPTIDE neoantigen designated as SEQ ID NO: 19,976.

Expression of the antigen, presence of the somatic mutation in the gene associated with the antigen, or expression of the HLA Class I subtype comprised in the antigen can be determined by any means known in the art.

Expression or presence of the antigen can be determined at the RNA or protein level by any means known in the art. Exemplary methods include, but are not limited to RNASeq, microarray, PCR, Nanostring, in situ hybridization (ISH), Mass spectrometry, or immunohistochemistry (IHC). Thresholds for positivity of gene expression is established by several methods, including: (1) predicted probability of presentation of the epitope by the HLA allele at various gene expression levels, (2) correlation of gene expression and HLA epitope presentation as measured by mass spectrometry, and/or (3) clinical benefits of ABP-based immunotherapy attained for patients expressing the genes at various levels.

For example, presence of the somatic mutation associated with the antigen can be determined by sequencing. In some embodiments, polynucleotides are isolated from the biological sample and sequenced. The polynucleotides can comprise DNA. The polynucleotides can comprise cDNA. The polynucleotides can comprise RNA. The sequencing can comprise whole exome sequencing, whole genome sequencing, targeted sequencing of a panel of cancer genes, or targeted sequencing of a single cancer gene. Exemplary gene panels include, but are not limited to FoundationOne, FoundationOne CDx, Guardant 360, Guardant OMNI, and MSK IMPACT. Presence of the somatic mutation associated with the antigen can also be determined by PCR based assays such as Cobas® KRAS Mutation Test. Presence of the somatic mutation associated with the antigen can also be determined by mass-spec based assays such as MassARRAY, described in Ibarrola-Villava, Maider et al. “Determination of somatic oncogenic mutations linked to target-based therapies using MassARRAY technology.” Oncotarget vol. 7, 16 (2016): 22543-55. doi:10.18632/oncotarget. 8002, which is hereby incorporated by reference in its entirety.

For example, presence of the HLA Class I subtype in the subject or biological sample of the subject can be determined by sequencing, e.g., next generation sequencing (NGS) of the HLA genes and analysis with a bioinformatic tool such as OptiType, standard sequence-specific oligonucleotide (SSO) and sequence-specific primer (SSP) technologies, or any other methods known in the art.

Combination Therapies

In some embodiments, an ABP and/or cell provided herein is administered with at least one additional therapeutic agent. Any suitable additional therapeutic agent may be administered with an ABP and/or cell provided herein. In some embodiments, the additional therapeutic agent is an ABP.

Diagnostic Methods

Also provided are methods for predicting and/or detecting the presence of a given HLA-PEPTIDE on a cell from a subject. Such methods may be used, for example, to predict and evaluate responsiveness to treatment with an ABP and/or cell provided herein.

In some embodiments, a blood or tumor sample is obtained from a subject and the fraction of cells expressing HLA-PEPTIDE is determined. In some aspects, the relative amount of HLA-PEPTIDE expressed by such cells is determined. The fraction of cells expressing HLA-PEPTIDE and the relative amount of HLA-PEPTIDE expressed by such cells can be determined by any suitable method. In some embodiments, flow cytometry is used to make such measurements. In some embodiments, fluorescence assisted cell sorting (FACS) is used to make such measurement. See Li et al., J. Autoimmunity, 2003, 21:83-92 for methods of evaluating expression of HLA-PEPTIDE in peripheral blood.

In some embodiments, detecting the presence of a given HLA-PEPTIDE on a cell from a subject is performed using immunoprecipitation and mass spectrometry. This can be performed by obtaining a tumor sample (e.g., a frozen tumor sample) such as a primary tumor specimen and applying immunoprecipitation to isolate one or more peptides. The HLA alleles of the tumor sample can be determined experimentally or obtained from a third party source. The one or more peptides can be subjected to mass spectrometry (MS) to determine their sequence(s). The spectra from the MS can then be searched against a database. An example is provided in the Examples section below.

In some embodiments, predicting the presence of a given HLA-PEPTIDE on a cell from a subject is performed using a computer-based model applied to the peptide sequence and/or RNA measurements of one or more genes comprising that peptide sequence (e.g., RNA seq or RT-PCR, or nanostring) from a tumor sample. The model used can be as described in international patent application no. PCT/US2016/067159, herein incorporated by reference, in its entirety, for all purposes.

Kits

Also provided are kits comprising an ABP and/or cell provided herein. The kits may be used for the treatment, prevention, and/or diagnosis of a disease or disorder, as described herein.

In some embodiments, the kit comprises a container and a label or package insert on or associated with the container. Suitable containers include, for example, bottles, vials, syringes, and IV solution bags. The containers may be formed from a variety of materials, such as glass or plastic. The container holds a composition that is by itself, or when combined with another composition, effective for treating, preventing and/or diagnosing a disease or disorder. The container may have a sterile access port. For example, if the container is an intravenous solution bag or a vial, it may have a port that can be pierced by a needle. At least one active agent in the composition is an ABP provided herein. The label or package insert indicates that the composition is used for treating the selected condition.

In some embodiments, the kit comprises (a) a first container with a first composition contained therein, wherein the first composition comprises an ABP and/or cell provided herein; and (b) a second container with a second composition contained therein, wherein the second composition comprises a further therapeutic agent. The kit in this embodiment can further comprise a package insert indicating that the compositions can be used to treat a particular condition, e.g., cancer.

Alternatively, or additionally, the kit may further comprise a second (or third) container comprising a pharmaceutically-acceptable excipient. In some aspects, the excipient is a buffer. The kit may further include other materials desirable from a commercial and user standpoint, including filters, needles, and syringes.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B(1992).

### Example 1: Identification of Shared HLA-PEPTIDE Neoantigens

We identified shared HLA-PEPTIDE neoantigens using a series of steps. We obtained a list of common driver mutations classified as “confirmed somatic” from the COSMIC database. For each mutation, we generated candidate neoepitopes (8 to 11-mer peptides), used a TPM of 100, and ran our EDGE prediction model (a model trained on HLA presented peptides sequenced by MS/MS, as described in U.S. Pat. No. 10,055,540, US Application Pub. No. US20200010849A1, international patent application publications WO/2018/195357 and WO/2018/208856, U.S. application Ser. No. 16/606,577, and international patent application PCT/US2020/021508, each herein incorporated by reference, in their entirety, for all purposes) across all modeled HLA alleles. Note that each peptide contained at least one mutant amino acid and was not a self-peptide. We then recorded any peptide with an HLA allele that has an EDGE score >0.001. The results are shown in Table A. A total of 10261 shared HLA-PEPTIDE neoantigen sequences were thus identified and are described in SEQ ID NO: 10,755-21,015. The corresponding HLA allele(s) for each sequence are shown.

The initial list provided in Table A was further analyzed for level of neoantigen/HLA prevalence in the patient population. “Antigen/HLA prevalence” or “is calculated as the frequency of a given mutation “(A)” in a given population multiplied by the frequency of an HLA allele “(B)” in the given population. Antigen/HLA prevalence can also refer to mutation/HLA prevalence or neoantigen/HLA prevalence. As part of this analysis, for each mutation, its (A) frequency was obtained across common tumor types in TCGA and recorded at its highest frequency amongst tumor types. (B) For each HLA allele in EDGE, the HLA allele frequency TCGA (a predominantly Caucasian population) was recorded. HLA allele frequencies are described in more detail in Shukla, S. A. et al. (Nat. Biotechnol. 33, 1152-1158 2015), herein incorporated by reference for all purposes. The neoantigen/HLA prevalence was calculated as (A) multiplied by (B). Any restricted peptide/HLA pair in Table A that is >0.1% prevalence using this methodology is identified with a “Most Common 1” (2387/10261).

Additionally, we characterized the prevalence of cancer driver mutations across a large cohort of patient samples representative of the advanced cancer patient population relevant for potential clinical studies. EDGE prediction was performed using the publicly-released AACR Genie v4.1 dataset, which has over 40,000 patients sequenced on NGS cancer gene panels ranging from 50 to 500 genes, from major academic cancer centers including Dana-Farber, Johns Hopkins, MD Anderson, MSKCC, and Vanderbilt. We selected base substitution and indel mutations in lung, microsatellite stable colon, and pancreatic cancers, and required coverage across multiple gene panels. We analyzed each neoantigen peptide paired with each of greater than 90 Class I HLA alleles covered in our EDGE antigen presentation prediction model and recorded the epitopes and corresponding HLA alleles with an EDGE probability of HLA presentation score of >0.001. We then determined the neoantigen/HLA prevalence of those peptide with an EDGE score >0.001, calculated as A*B, where A is the highest frequency of the mutation amongst the three tumor types and B is the HLA allele frequency. We used HLA allele frequencies representative of the population in the USA by examining HLA alleles from the TCGA population and tabulating the frequency for each HLA allele (Shukla, S. A. et al.). Peptides and corresponding HLA alleles that demonstrated a neoantigen/HLA prevalence >0.01% from the analysis are described in SEQ ID NO:21,016-29,357 and referred to as AACR GENIE Results.

### Example 2: Validation of Shared HLA-PEPTIDE Neoantigen Presentation

Mass spectrometry (MS) validation of candidate shared HLA-PEPTIDE neoantigens was performed using targeted mass spectrometry methods. Nearly 500 frozen resected lung, colorectal and pancreatic tumor samples were homogenized and used for both RNASeq transcriptome sequencing and immunoprecipitation of the HLA/peptide complexes. A peptide target list was generated for each sample by analysis of the transcriptome, whereby recurrent cancer driver mutations, as defined in the AACR Genie v4.1 dataset, were identified and RNA expression levels assessed. The EDGE model of antigen presentation was then applied to the mutation sequence and expression data to prioritize peptides for the targeting list. The peptides from the HLA molecules were eluted and collected using size exclusion to isolate the presented peptides prior to mass spectrometry. Synthetic heavy labeled peptide with the same amino acid sequence was co-loaded with each sample for targeted mass spectrometry. Both coelution of the heavy labeled peptide with the experimental peptide and analysis of the fragmentation pattern we were used to validate a candidate epitope. Mass spectrometry analysis methods are described in more detail in Gillete et al. (Nat Methods. 2013 January; 10(1):28-34), herein incorporated by reference in its entirety for all purposes.

Shared HLA-PEPTIDE neoantigens from driver mutations validated in this manner with sufficient prevalence for further consideration are summarized in Table 5A below, along with sample tumor type and associated HLA alleles.

TABLE 5A
Expression of MS-validated HLA-PEPTIDE neoantigens

| Patient | Tumor | Presenting | Gene_ | Peptide Elution | Targeted |
|---|---|---|---|---|---|
| ID | Type | HLA* | Mutation | Time (min.) | Peptide |
| A0002082 | CRC | HLA-A*03:01 | CTNNB1_S45P | 17.2 | TTAPPLSGK |
| A0001877 | Lung | HLA-A*02:01 | KRAS_G12C | 46.7 | KLVVVGACGV |
| A0002129 | Lung | HLA-A*02:01 | KRAS_G12C | 46.5 | KLVVVGACGV |
| A0001947 | CRC | HLA-A*11:01 | KRAS_G12D | 12 (pep. #1) | VVGADGVGK |
| A0001947 | CRC | HLA-A*11:01 | KRAS_G12D | 23.4 (pep. #2) | VVVGADGVGK |
| A0001474 | Gastric | HLA-A*11:01 | KRAS_G12V | 30.4 | VVVGAVGVGK |
| A0001730 | Pancreatic | HLA-A*11:01 | KRAS_G12V | 18.4 (pep. #1) | VVGAVGVGK |
| A0001730 | Pancreatic | HLA-A*11:01 | KRAS_G12V | 31.6 (pep. #2) | VVVGAVGVGK |
| A0001896 | Lung | HLA-C*01:02 | KRAS_G12V | 23.6 | AVGVGKSAL |
| A0001966 | CRC | HLA-A*03:01 | KRAS_G12V | 27.8 | VVVGAVGVGK |
| A0001973 | Ovarian | HLA-A*24:02 | TP53_K132N | 97.7 | TYSPALNNMF |
| A0001983 | Ovarian | HLA-A*02:01 | CTNNB1_S37Y | 50.9 | YLDSGIHYGA |
| A0000799 | CRC | HLA-B*08:01 | CHD4_K73fs | 43.3 | TVRAATIL |

*When the same peptide was predicted to be presented by multiple HLA alleles for a patient and detected by MS/MS, it was inferrec 1 to be presented by the highest scoring HLA allele by EDGE or both alleles if the scores were sufficiently close

Selected HLA-PEPTIDE neoantigens were also validated by in vitro assay. Briefly, cell lines were engineered to express a single specific HLA alleles and inducibly express a candidate shared neoantigen according to methods described below.

Materials and Methods for validation of selected HLA-PEPTIDE neoantigens by in vitro assay.

Single HLA allele expressing K562 cell lines were created by traditional transfection methods using reagent kits and the instructions provided.

To create virus particles for transduction of the HLA genes into K562 cells the plasmids were transfection into Phoenix-ampho cells. Phoenix-ampho cells were introduced into 6 well plates at a density of 5×10⁵ cells per well and incubated at 37C overnight prior to transfection. 10 ug of purified DNA was mixed with 10 uL Plus Reagent and brought to 100 uL with pre-warmed Opti-MEM media. Lipofectamine reagent was prepared by mixing 8 uL of Lipofectamine with 92 uL of the pre-warmed Opti-MEM. Both mixtures were incubated at room temperature for 15 prior to mixing the 100 uL of Lipofectamine reagent with the 100 uL of DNA solution and allowing the combined solution to incubate at room temperature for another 15 min. The Phoenix-ampho cells were washed gently by aspirating the media and adding 6 mL of pre-warmed Opti-MEM media to wash the cells. The media was removed from the plated cells. 800 uL of the pre-warmed Opti-MEM was added to the DNA/Lipofectamine mixture to make 1 mL and that solution was added to the plated cells. After the plate was incubated for 3 hrs at 37C, 3 mL of complete media was added and the cells were incubated overnight at 37C. The complete media was exchanged after the incubation and the cells incubated for another 2 days. Virus particles were collected after the supernatant was passed through a 45 um filter into a new 6 well plate. 20 uL of Plus reagent and 8 uL of Lipofectamine was added to each well with a 15 min room temperature incubation after each addition.

K562 cells were suspended complete media at a concentration of 5×10⁶ per mL. 100 uL of K562 cells were added to each well of the 6 well plate containing the virus particles. The plate was centrifuged at 700×g for 20 min and the cells were incubated for 6 hrs at 37C. Cells and virus were collected and transferred to T25 flasks with the addition of 7 mL of complete media. The cells were incubated for 3 days prior to a media change which included selection with Puromyocin antibiotic. Live cells were collected and passaged to create stocks of K562 cells expressing a single HLA allele. Overall 25 of these cell lines were created, each with a different HLA expressed, to provide a reagent pool for future experiments.

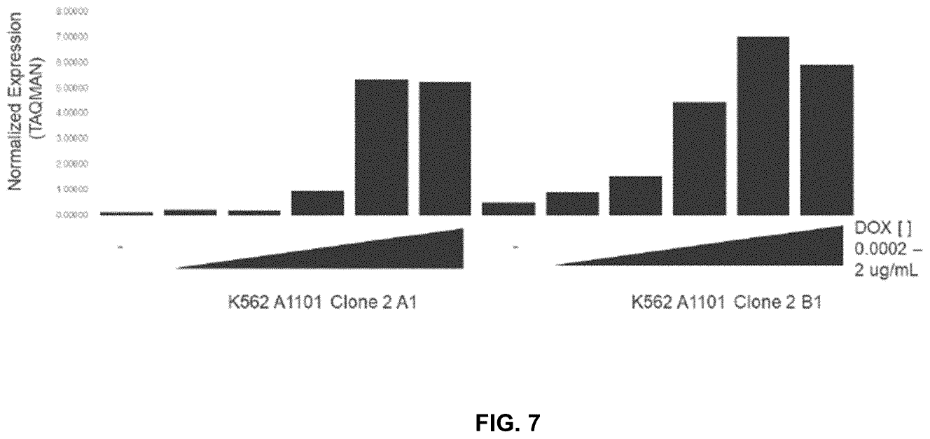

A shared neoantigen cassette was created to express 20 shared neoantigens with the mutation centered in a 25mer amino acid chain and was created with no linkers between the entries. This cassette was subcloned into a lentiviral Tet-One Inducible Expression vector system (Clontech) and lentivirus was produced in 293T cells by cotransfecting the shared neoantigen expression vector with ViraPower (Thermo) packaging plasmids according to manufacturer's specifications. Single HLA Allele expressing K562 cell lines were then transduced with this virus as described above and single cell clones were characterized for shared neoantigen expression. Briefly, expression of the shared neoantigen cassette was placed under control of a doxycycline (DOX)-controlled TRE3G promoter, where administration of DOX leads to expression of the neoantigens via stabilization of the Tet-On 3G transactivator protein that is constitutively expressed on the same plasmid. The TREG3 promoter—Tet-On 3G transactivator system allows titration of DOX to control the level of expression. As shown in FIG. 7, expression of a representative neoantigen increased as the concentration of DOX administered increased, demonstrating regulatable expression.

Cells containing both the single HLA allele and the shared neoantigen cassette were grown to ˜2.5×10⁸ cells and pelleted into 15 mL vials. Additionally, cells were plated in limited dilution to prepare single clones of the HLA/Cassette pairing. These single clones were tested to achieve a variety of expression levels of the cassette. Use of cell lines with differing expression levels of the cassette allows for analysis of the system at close to endogenous expression levels. Single clones were also grown to ˜2.5×10⁸ cells and pelleted into 15 mL vials. All pellets were washed 2× with cold PBS and frozen to allow for processing for mass spectrometry detection of HLA peptides. Expression levels of the HLA and the cassette was performed using SmartSeq or Tagman assays with appropriate probes.

For isolation of HLA peptides, each cell pellet was lysed with lysis buffer and centrifuged at 20,000×g for 1 hr to clarify the lysate and the HLA peptide complexes were enriched. Heavy peptides—peptides synthesized with amino acids containing isotopically heavy amino acids—were added to the peptides prior to analysis by MS to aid in confirmation of the identity of the peptides detected.

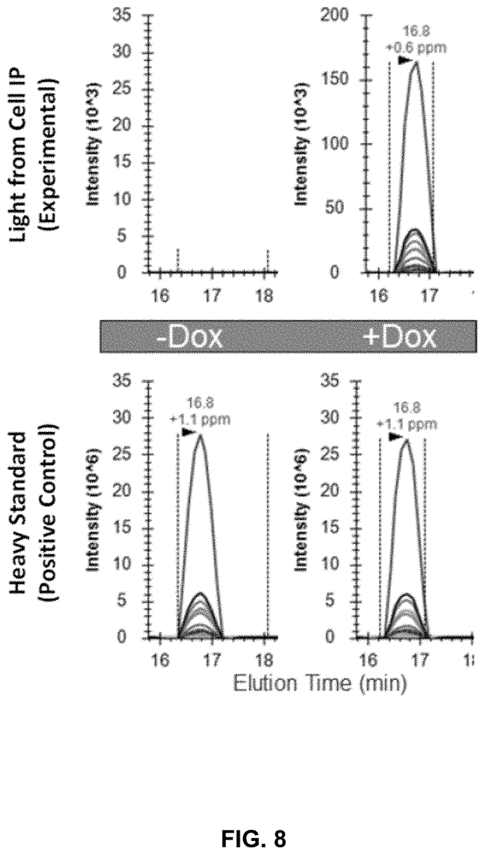

As shown in FIG. 8, a representative KRAS G12V peptide VVGAVGVGK was observed by mass-spectrometry in a HLA-A*11:01 expressing K562 cell line, in a DOX-dependent manner (FIG. 8, top panels). Detection of the heavy peptide control standard was equivalent (FIG. 8, bottom panels). Thus, validation of HLA-specific presentation of predicted neoantigens was demonstrated using the single-HLA K562 in vitro system.

The in vitro system described above was used to validate HLA-specific presentation of predicted neoantigens.

Results are shown in Table 5B, below.

TABLE 5B
Validated HLA-PEPTIDE neoantigens by in vitro assay

| | HLA | Peptide | Peptide |
|---|---|---|---|
| Gene | Type | Detected | sequence |
| CTNNB1_S45P | A*11:01 | 9-mer | TTAPPLSGK |
| CTNNB1_T41A | A*11:01 | 9-mer | ATAPSLSGK |
| KRAS_G12D | A*11:01 | 10-mer | VVVGADGVGK |
| KRAS_G12V | A*03:01 | 9-mer | VVGAVGVGK |
| KRAS_G12V | A*03:01 | 10-mer | VVVGAVGVGK |
| KRAS_G12V | A*11:01 | 9-mer | VVGAVGVGK |
| KRAS_G12V | A*11:01 | 10-mer | VVVGAVGVGK |
| KRAS_Q61R | A*01:01 | 10-mer | ILDTAGREEY |
| TP53_R213L | A*02:01 | 9-mer | YLDDRNTFL |

We further evaluated the MS data with respect to mutations for which peptides were not detected in order to assess the value of narrowly targeting patients with specific HLAs for treatment, e.g., requiring patients to have at least one validated or predicted HLA allele that presents a restricted peptide disclosed herein.

For example, in the case of KRAS, we counted the number of patient samples in which KRAS restricted peptides for particular HLA alleles were detected or not detected. (When the same peptide was predicted to be presented by multiple HLA alleles for a patient and detected by MS/MS, it was inferred to be presented by the highest scoring HLA allele by EDGE or both alleles if the scores were sufficiently close). Results are presented in Table 6. Based on these results, several common HLA alleles are not expected to present a given KRAS restricted peptide and these KRAS restricted peptide/HLA pairs can be excluded for purposes of selection criteria for immunotherapy design and patient selection in this instance. For example, Table 7 directed to selected HLA-PEPTIDE neoantigen targets for immunotherapy does not include predicted HLA-PEPTIDE neoantigen HLA-A*02:01_RAS G12D, on the basis that the restricted peptide was not detected in 17 samples tested, and likewise did not include G12V/A*02:01 on the basis that the peptide was not detected in 9 samples tested. In contrast, neoantigen/HLA pair G12D/A*11:01 was considered validated on the basis that the peptide was detected in ⅕ samples tested, and likewise G12V/A*11:01 was considered validated on the basis that the peptide was detected in 2/6 samples tested.

These results highlight the importance of identifying the relevant restricted peptide/HLA pairs for proper HLA type selection in patient selection for treatment with a shared neoantigen immunotherapy, such as that described in Table 7. Specifically, several common KRAS restricted peptide/HLA pairs were excluded for purposes of selection criteria in this case as the MS data suggested a shared HLA-PEPTIDE neoantigen ABP would unlikely provide a benefit to a patient with that predicted KRAS neoantigen/HLA pair (e.g., G12D/A*02:01 or G12V/A*02:01).

TABLE 6

| | | HLA Peptide | Number of tested |
|---|---|---|---|
| Gene | | confirmation | patient samples |
| mutation | HLA | by MS/MS | with MS/MS result |
| KRAS_G12A | HLA-A*02:01 | N | 2 |
| KRAS_G12A | HLA-A*02:06 | N | 1 |
| KRAS_G12A | HLA-A*03:01 | N | 3 |
| KRAS_G12A | HLA-B*27:05 | N | 1 |
| KRAS_G12A | HLA-B*35:01 | N | 1 |
| KRAS_G12A | HLA-B*41:02 | N | 1 |
| KRAS_G12A | HLA-B*48:01 | N | 1 |
| KRAS_G12A | HLA-C*08:03 | N | 1 |
| KRAS_G12C | HLA-A*02:01 | Y | 2 |
| KRAS_G12C | HLA-A*02:01 | N | 2 |
| KRAS_G12C | HLA-A*03:01 | N | 8 |
| KRAS_G12C | HLA-A*03:02 | N | 1 |
| KRAS_G12C | HLA-A*68:01 | N | 1 |
| KRAS_G12C | HLA-B*27:05 | N | 1 |
| KRAS_G12D | HLA-A*02:01 | N | 17 |
| KRAS_G12D | HLA-A*02:05 | N | 2 |
| KRAS_G12D | HLA-A*03:01 | N | 4 |
| KRAS_G12D | HLA-A*11:01 | Y | 1 |
| KRAS_G12D | HLA-A*11:01 | N | 4 |
| KRAS_G12D | HLA-A*26:01 | N | 2 |
| KRAS_G12D | HLA-A*31:01 | N | 4 |
| KRAS_G12D | HLA-A*68:01 | N | 3 |
| KRAS_G12D | HLA-B*07:02 | N | 4 |
| KRAS_G12D | HLA-B*08:01 | N | 1 |
| KRAS_G12D | HLA-B*13:02 | N | 1 |
| KRAS_G12D | HLA-B*15:01 | N | 5 |
| KRAS_G12D | HLA-B*27:05 | N | 2 |
| KRAS_G12D | HLA-B*35:01 | N | 2 |
| KRAS_G12D | HLA-B*37:01 | N | 1 |
| KRAS_G12D | HLA-B*38:01 | N | 2 |
| KRAS_G12D | HLA-B*40:01 | N | 3 |
| KRAS_G12D | HLA-B*40:02 | N | 3 |
| KRAS_G12D | HLA-B*44:02 | N | 1 |
| KRAS_G12D | HLA-B*44:03 | N | 4 |
| KRAS_G12D | HLA-B*48:01 | N | 1 |
| KRAS_G12D | HLA-B*50:01 | N | 1 |
| KRAS_G12D | HLA-B*57:01 | N | 1 |
| KRAS_G12D | HLA-C*01:02 | N | 1 |
| KRAS_G12D | HLA-C*02:02 | N | 1 |
| KRAS_G12D | HLA-C*03:03 | N | 3 |
| KRAS_G12D | HLA-C*03:04 | N | 2 |
| KRAS_G12D | HLA-C*04:01 | N | 8 |
| KRAS_G12D | HLA-C*05:01 | N | 2 |
| KRAS_G12D | HLA-C*07:04 | N | 1 |
| KRAS_G12D | HLA-C*08:02 | N | 2 |
| KRAS_G12D | HLA-C*08:03 | N | 1 |
| KRAS_G12D | HLA-C*16:01 | N | 1 |
| KRAS_G12D | HLA-C*17:01 | N | 1 |
| KRAS_G12R | HLA-B*41:02 | N | 1 |
| KRAS_G12R | HLA-C*07:04 | N | 1 |
| KRAS_G12V | HLA-A*02:01 | N | 9 |
| KRAS_G12V | HLA-A*02:05 | N | 1 |
| KRAS_G12V | HLA-A*02:06 | N | 1 |
| KRAS_G12V | HLA-A*03:01 | Y | 1 |
| KRAS_G12V | HLA-A*03:01 | N | 4 |
| KRAS_G12V | HLA-A*11:01 | Y | 2 |
| KRAS_G12V | HLA-A*11:01 | N | 4 |
| KRAS_G12V | HLA-A*25:01 | N | 3 |
| KRAS_G12V | HLA-A*26:01 | N | 2 |
| KRAS_G12V | HLA-A*30:01 | N | 2 |
| KRAS_G12V | HLA-A*31:01 | N | 2 |
| KRAS_G12V | HLA-A*32:01 | N | 1 |
| KRAS_G12V | HLA-A*68:02 | N | 1 |
| KRAS_G12V | HLA-B*07:02 | N | 6 |
| KRAS_G12V | HLA-B*08:01 | N | 1 |
| KRAS_G12V | HLA-B*13:02 | N | 2 |
| KRAS_G12V | HLA-B*14:02 | N | 1 |
| KRAS_G12V | HLA-B*15:01 | N | 2 |
| KRAS_G12V | HLA-B*27:05 | N | 2 |
| KRAS_G12V | HLA-B*39:01 | N | 1 |
| KRAS_G12V | HLA-B*40:01 | N | 1 |
| KRAS_G12V | HLA-B*40:02 | N | 1 |
| KRAS_G12V | HLA-B*41:02 | N | 1 |
| KRAS_G12V | HLA-B*44:05 | N | 1 |
| KRAS_G12V | HLA-B*50:01 | N | 1 |
| KRAS_G12V | HLA-B*51:01 | N | 1 |
| KRAS_G12V | HLA-C*01:02 | Y | 1 |
| KRAS_G12V | HLA-C*01:02 | N | 1 |
| KRAS_G12V | HLA-C*03:03 | N | 1 |
| KRAS_G12V | HLA-C*03:04 | N | 2 |
| KRAS_G12V | HLA-C*08:02 | N | 2 |
| KRAS_G12V | HLA-C*14:02 | N | 1 |
| KRAS_G12V | HLA-C*17:01 | N | 2 |
| KRAS_G13D | HLA-A*02:01 | N | 2 |
| KRAS_G13D | HLA-B*07:02 | N | 1 |
| KRAS_G13D | HLA-B*08:01 | N | 1 |
| KRAS_G13D | HLA-B*35:01 | N | 1 |
| KRAS_G13D | HLA-B*35:03 | N | 1 |
| KRAS_G13D | HLA-B*35:08 | N | 1 |
| KRAS_G13D | HLA-B*38:01 | N | 1 |
| KRAS_G13D | HLA-C*04:01 | N | 3 |
| KRAS_Q61H | HLA-A*01:01 | N | 1 |
| KRAS_Q61H | HLA-A*02:01 | N | 2 |
| KRAS_Q61H | HLA-A*23:01 | N | 2 |
| KRAS_Q61H | HLA-A*29:01 | N | 1 |
| KRAS_Q61H | HLA-A*30:02 | N | 1 |
| KRAS_Q61H | HLA-A*33:01 | N | 1 |
| KRAS_Q61H | HLA-A*68:01 | N | 1 |
| KRAS_Q61H | HLA-B*07:02 | N | 1 |
| KRAS_Q61H | HLA-B*08:01 | N | 1 |
| KRAS_Q61H | HLA-B*18:01 | N | 1 |
| KRAS_Q61H | HLA-B*35:01 | N | 1 |
| KRAS_Q61H | HLA-B*38:01 | N | 1 |
| KRAS_Q61H | HLA-B*40:01 | N | 1 |
| KRAS_Q61H | HLA-B*44:02 | N | 1 |
| KRAS_Q61H | HLA-C*03:04 | N | 1 |
| KRAS_Q61H | HLA-C*05:01 | N | 2 |
| KRAS_Q61H | HLA-C*08:02 | N | 1 |

### Example 3: Selection of Shared HLA-PEPTIDE Neoantigens for Immunotherapy

A selection of clinically useful HLA-PEPTIDE neoantigen targets for immunotherapy (“GO-005”) containing 20 shared HLA-PEPTIDE neoantigens was constructed. Table 7 describes features of the HLA-PEPTIDE neoantigens selected for the selection. Shared HLA-PEPTIDE neoantigens directly detected on the surface of tumor cells by mass spectrometry, as described above in Table 5A, were included in the cassette and the HLA of the epitope was added to the eligible HLA list for the mutations. HLA-PEPTIDE neoantigens not independently verified as being presented in our assays were considered validated and added if there was compelling literature evidence of tumor presentation (e.g., tumor-infiltrating lymphocytes (TIL) recognizing the neoantigen). KRAS G12D presented by HLA-C*08:02 was considered validated and added based on literature evidence of adoptive cell therapy targeting this HLA-PEPTIDE neoantigen causing tumor regression in a patient with CRC (Tran et al. N Engl J Med. 2016 Dec. 8; 375(23): 2255-2262). HLA-PEPTIDE neoantigens with validated HLA alleles occupied 6 out of 20 slots.

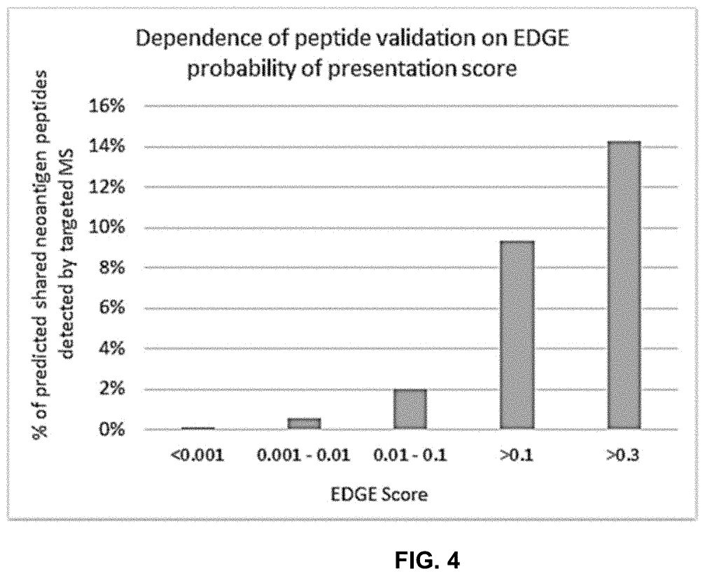

Additional, rarer HLA-PEPTIDE neoantigens predicted to be presented by tumor cells, but not yet validated by MS, were used to complement the initial set. Mutations with high EDGE scores were prioritized for inclusion as predicted HLA-PEPTIDE neoantigens given the strong dependence we observed between EDGE score and probability of detection of candidate shared HLA-PEPTIDE neoantigen peptides by targeted mass spectrometry (MS) validation experiments described herein. Results showing the correlation between EDGE score and the probability of detection of candidate shared HLA-PEPTIDE neoantigen peptides by targeted MS are shown in FIG. 4. Specifically, predicted HLA-PEPTIDE neoantigens with an EDGE HLA presentation score of at least 0.3 and the highest cumulative neoantigen/HLA prevalence across NSCLC, CRC and Pancreatic cancer were included in the selection. Combined HLA frequency was required to be at least 5-10% (e.g., there are 11% of the American population harboring HLA alleles B1501 or B1503). Of note, KRAS and NRAS harbors the same sequence around codons 12, 13, and 61. Validated HLAs, predicted HLAs with an EDGE score of at least 0.3, the mean EDGE score of the predicted HLAs, and neoantigen/HLA prevalence in the three cancer populations are also presented in Table 7.

Table 7 (below) depicts 20 exemplary shared HLA-PEPTIDE neoantigens comprising a cancer-related mutation and a particular HLA Class I allele, based on EDGE Score and prevalence in cancer patient populations. The exemplary shared HLA-PEPTIDE neoantigens are particularly useful targets for cancer immunotherapy, e.g., by treatment with an ABP that selectively binds the HLA-PEPTIDE neoantigen.

TABLE 7
Selected Shared HLA-PEPTIDE neoantigens

| | | | | Neo-Ag/ | Neo-Ag/ | Neo-Ag/ |
|---|---|---|---|---|---|---|
| | | | | HLA | HLA | HLA |
| | | Validated | Predicted | Prevalence | Prevalence | Prevalence |
| Slot | Mutation | HLA | HLA | in Lung | in CRC | in Pancreas |
| 1 | KRAS_G13D | A1101 | C0802 | 0.07% | 0.39% | 0.06% |
| 2 | KRAS_Q61K | A0101 | — | 0.05% | 0.37% | 0.00% |
| | NRAS_Q61K | | | | | |
| 3 | TP53_R249M | — | B3512, | 0.04% | 0.00% | 0.00% |
| | | | B3503, | | | |
| | | | B3501 | | | |
| 4 | CTNNB1_S45P | A0301, | A6801, | 0.13% | 0.00% | 0.00% |
| | | A1101 | A0302, | | | |
| 5 | CTNNB1_S45F | A1101 | A0301, | 0.08% | 0.27% | 0.00% |
| | | | A6801 | | | |
| 6 | ERBB2_Y772_A775dup | — | B1801 | 0.11% | 0.00% | 0.00% |
| 7 | KRAS_G12D | A1101, | — | 0.79% | 2.28% | 5.45% |
| | NRAS_G12D | A0301, | | | | |
| | | C0802 | | | | |
| 8 | KRAS_Q61R | A0101 | — | 0.06% | 0.33% | 0.47% |
| | NRAS_Q61R | | | | | |
| 9 | CTNNB1_T41A | A1101 | A0301, | 0.00% | 0.27% | 0.00% |
| | | | A0302, | | | |
| | | | B1510, | | | |
| | | | C0303, | | | |
| | | | C0304 | | | |
| 10 | TP53_K132N | A2402 | A2301 | 0.04% | 0.00% | 0.00% |
| 11 | KRAS_G12A | A0301, | — | 0.58% | 0.49% | 0.00% |
| | | A1101 | | | | |
| 12 | KRAS_Q61L | — | A0101 | 0.22% | 0.08% | 0.00% |
| | NRAS_Q61L | | | | | |
| 13 | TP53_R213L | A0201 | A0207, | 0.09% | 0.18% | 0.00% |
| | | | C0802 | | | |
| 14 | BRAF_G466V | — | B1501, | 0.03% | 0.05% | 0.00% |
| | | | B1503 | | | |
| 15 | KRAS_G12V | A0301, | A0302 | 2.56% | 3.43% | 9.89% |
| | | A1101, | | | | |
| | | C0102 | | | | |
| 16 | KRAS_Q61H | A0101 | — | 0.42% | 0.28% | 0.91% |
| | NRAS_Q61H | | | | | |
| 17 | CTNNB1_S37F | A0101 | A2301, | 0.29% | 0.00% | 0.00% |
| | | | A2402 | | | |
| | | | B1510, | | | |
| | | | B3906 | | | |
| | | | C0501, | | | |
| | | | C1402 | | | |
| | | | C1403 | | | |
| 18 | TP53_S127Y | — | A1101, | 0.04% | 0.00% | 0.00% |
| | | | A0301 | | | |
| 19 | TP53_K132E | — | A2402, | 0.00% | 0.05% | 0.00% |
| | | | C1403, | | | |
| | | | A2301 | | | |
| 20 | KRAS_G12C | A0201, | — | 5.00% | 1.46% | 0.63% |
| | NRAS_G12C | A0301, | | | | |
| | | A1101 | | | | |

Additionally, we determined the total population of patients with at least one HLA allele identified (i.e., either validated or predicted) to present at least one shared neoantigen from Table 7 (i.e., the HLA-PEPTIDE neoantigen comprising both the mutation and the HLA allele, referred to herein as GO-005 targeted patient population) and compared it to the population of patients with the mutations (agnostic of whether the patient had the identified allele). To estimate the GO-005 targeted patient population, we collected patient mutation data from AACR Genie. As such patients do not have matching HLA alleles, we sampled HLA alleles from the TCGA population and paired it to the AACR Genie dataset. Then given a tumor type, any patient from AACR Genie with matching both mutation and HLA was labeled positive, and any patient that doesn't meet the criteria was labeled negative. The percent positives give the overall addressable patient population, per tumor type, in Table 8.

It can be readily appreciated from Table 8 that only a subset of patients who carry a particular mutation also carry the HLA allele likely to present that mutation as a HLA-PEPTIDE neoantigen. Patients with the mutation, but without the appropriate HLA allele are less likely to benefit from therapy. As an example, whereas an estimated ˜60% of pancreatic cancer patients carry appropriate mutations/neoantigens, more than 2 out of 3 of these patients do not carry the corresponding HLA allele(s). Therefore, an ABP-based immunotherapy strategy that considers the relevant mutation and HLA allele pairs as proposed will target primarily those patients who may benefit. Thus, consideration of epitope presentation by validated or high-scoring predicted HLA is an important step in determining the potential efficacy of a shared HLA-PEPTIDE neoantigen ABP.

TABLE 8
Neoantigen/HLA Prevalence in Target Populations

| | MSS CRC | Pancreas | Lung |
|---|---|---|---|
| GO-005 Targeted Patient | 9.0% | 17.4% | 10.6% |
| Population (cumulative | | | |
| neoantigen/HLA prevalence) | | | |
| HLA agnostic patient population | 35.1% | 60.8% | 32.0% |
| (mutation frequency only) | | | |

### Example 4: Evaluation of Immune Response Induction by Shared HLA-PEPTIDE Neoantigens

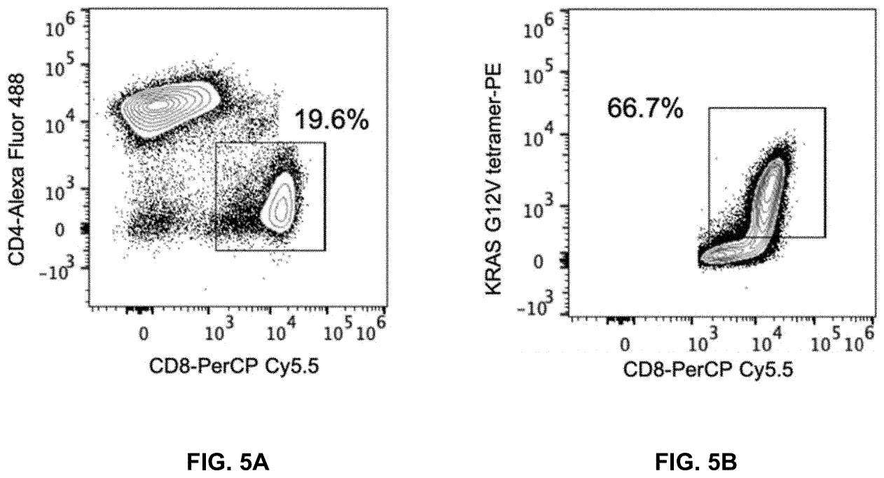

We evaluated whether HLA-PEPTIDE neoantigens induce an immune response in patients. We obtained dissociated tumor cells from a patient with lung adenocarcinoma. Tumor cells were sequenced to determine the patient's HLA and identify mutations. The patient expressed HLA-A*11:01 and we identified the KRAS G12V mutation in the tumor. Simultaneously, we sorted and expanded CD45+ cells from the tumor which represent tumor infiltrating lymphocytes (TIL). Expanded TILs were stained with mutated peptide HLA-A*11:01 tetramers to assess immunogenicity of this mutation in the patient. FIG. 5 shows the flow cytometry gating strategy on CD8+ cells (FIG. 5A) and the staining of CD8+ cells by KRAS-G12V/HLA-A*11:01 tetramer (FIG. 5B). A large portion (greater than 66%) of CD8+ T cells demonstrate binding to the KRAS G12V:HLA*1101 tetramer, indicating the ability of CD8+ T cells to recognize the HLA-PEPTIDE neoantigen and indicating a pre-existing immune response to the HLA-PEPTIDE neoantigen.

In addition, CD8+ cells in the expanded TILs were labeled with the KRAS-G12V/HLA-A*11:01 tetramer and sorted. The TCRs were sequenced using 10× Genomics single cell resolution paired immune TCR profiling approach (Chromium Single Cell A Chip Kit, Chromium Single Cell 5′ Library & Gel Bead Kit, Chromium Single Cell 5′ Library Construction kit, Chromium Single Cell 5′ Feature Barcode Library Kit [10× Genomics]). Sequencing reads were processed through the 10× provided software Cell Ranger. Sequencing reads were tagged with a Chromium cellular barcodes and UMIs, which are used to assemble the V(D)J transcripts cell by cell. The assembled contigs for each cell were then annotated by mapping the assembled contigs to the Ensemble v87 V(D)J reference sequences. Clonotypes were defined as alpha, beta chain pairs containing unique CDR3 sequences. Clonotypes were filtered for single alpha and single beta chain pairs present at frequency above 2 cells to yield the final list of clonotypes per target peptide in a specific donor. As shown in Table 1B and 1D, multiple TCR sequences were identified for KRAS-G12V/HLA-A*11:01, including to different G12V epitopes. The results demonstrate that neoantigen-specific TCRs can be identified from subject samples, such as TILs.

### Example 5: Selection of Shared HLA-PEPTIDE Neoantigens and Patient Populations for HLA-PEPTIDE Neoantigen-Specific Immunotherapy

An ABP (e.g. a TCR), or a cell engineered to express an ABP (e.g., a T cell, such as a autologous T cell, engineered to express an antigen/neoantigen specific TCR), to a HLA-PEPTIDE neoantigen as described in Table 7, Table A, the AACR GENIE Results, or SEQ ID NOs 29358-29364 described herein (SEQ ID NOs: 10,755-29,364) is administered to a cancer patient. The ABP is administered to a patient, e.g., to treat cancer. In certain instances the patient is selected, e.g., using a companion diagnostic or a commonly use cancer gene panel NGS assay such as FoundationOne, FoundationOne CDx, Guardant 360, Guardant OMNI, or MSK IMPACT. Exemplary patient selection criteria are described below.

### Patient Selection

Patient selection for ABP administration is performed by consideration of tumor gene expression, somatic mutation status, and patient HLA type. Specifically, a patient is considered eligible for the ABP-based immunotherapy therapy if the patient has cancer, and if:
(a) one or more cells of the patient expresses or is known to express an HLA Class I molecule as described in any one of SEQ ID NOs:10,755 to 29,364. In such cases, the patient may be administered an ABP that targets a HLA-PEPTIDE neoantigen described herein, such that the HLA-PEPTIDE neoantigen comprises the same HLA Class I molecule expressed by the one or more cells of the patient. By way of example only, a patient is considered eligible for ABP-based immunotherapy by administration of an ABP that selectively binds to RAS G12D neoantigen HLA-A*11:01_VVVGADGVGK (“SNA30”) if one or more cells of the patient expresses or is known to express HLA-A*11:01.
(b) one or more cells of the patient expresses or is known to express an HLA Class I molecule as described in any one of SEQ ID NOs:10,755 to 29,364, and the cancer expresses or is predicted to express a gene associated with a somatic mutation. By way of example only, a patient is considered eligible for ABP-based immunotherapy by administration of an ABP that selectively binds to RAS G12D neoantigen HLA-A*11:01_VVVGADGVGK (“SNA30”) if one or more cells of the patient expresses or is known to express HLA-A*11:01 and the cancer expresses or is predicted to express KRAS.
(c) one or more cells of the patient expresses or is known to express an HLA Class I molecule as described in any one of SEQ ID NOs:10,755 to 29,364, and the patient tumor or tumor nucleic acid carries the somatic mutation associated with the SEQ ID NO. By way of example only, a patient is considered eligible for ABP-based immunotherapy by administration of an ABP that selectively binds to RAS G12D neoantigen HLA-A*11:01_VVVGADGVGK (“SNA30”) if one or more cells of the patient expresses or is known to express HLA-A*11:01 and a tumor sample from the patient harbors the RAS G12D somatic mutation.
(d) Same as (b) or (c), but also requiring that the patient tumor expresses the gene with the mutation above a certain threshold (e.g., 1 TPM or 10 TPM), or
(e) Same as (b) or (c), but also requiring that the patient tumor expresses the mutation above a certain threshold (e.g., at least 1 mutated read observed at the level of RNA)
(f) Same as (b) or (c), but also requiring both additional criteria in (c) and (d)
(g) Any of the above, but also optionally requiring that loss of the presenting HLA allele is not detected in the tumor

Gene expression may be measured at the RNA or protein level by methods including, but not limited to RNASeq, microarray, PCR, Nanostring, ISH, Mass spectrometry, or IHC. Thresholds for positivity of gene expression is established by several methods, including: (1) predicted probability of presentation of the epitope by the HLA allele at various gene expression levels, (2) correlation of gene expression and HLA epitope presentation as measured by mass spectrometry, and/or (3) clinical benefits of ABP-based immunotherapy attained for patients expressing the genes at various levels.

Somatic mutational status may be assessed by any of the established methods, including exome sequencing (NGS DNASeq), targeted exome sequencing (panel of genes), transcriptome sequencing (RNASeq), Sanger sequencing, PCR-based genotyping assays (e.g., Tagman or droplet digital PCR), Mass-spectrometry based methods (e.g., by Sequenom), next generation sequencing, massively parallel sequencing, or any other method known to those skilled in the art.

### Example 6: Identification of TCRs that Bind HLA-PEPTIDE Target Neoantigens

### Methods

Peripheral blood mononuclear cells (PBMCs) were obtained by processing leukapheresis samples from healthy donors. Frozen PBMCs were thawed and enriched for different subsets of T cells through negative depletion using the following magnetic-activated cell sorting (MACS) systems (Miltenyi Biotech), as indicated below: (i) Pan T Cell Isolation Kit to enrich for naïve and memory CD4 and CD8 T cells; or a (ii) Naïve CD8 T Cell Isolation Kit & CD4 depletion kit to enrich for naïve CD8 T cells. Enriched T cells were labeled either with a single neoantigen-MHC tetramer or a pool of neoantigen-MHC tetramers of interest, as indicated below, as well as stained with live/dead and lineage markers and sorted by FACS. In addition, in some experiments the enriched T cells were labeled with a peptide-MHC tetramer containing the wildtype peptide corresponding to the neoantigen(s) of interest. Sorted T cells were polyclonally expanded with feeder cells and IL-2 for 2-3 weeks.

Following polyclonal expansion, the resulting cells were either:

a. labeled again with a target peptide-MHC tetramer and resorted (bulk resort) for peptide-MHC tetramer labeled cells

b. stimulated with neoantigen (10 μM) loaded PBMCs (or a DMSO control) and resorted (bulk resort) for CD137 upregulation after a day of stimulation

Cells isolated after resort were sequenced using 10× Genomics single cell resolution paired immune TCR profiling approach (Chromium Single Cell A Chip Kit, Chromium Single Cell 5′ Library & Gel Bead Kit, Chromium Single Cell 5′ Library Construction kit, Chromium Single Cell 5′ Feature Barcode Library Kit [10× Genomics]). Sequencing reads were processed through the 10× provided software Cell Ranger. Sequencing reads were tagged with a Chromium cellular barcodes and UMIs, which are used to assemble the V(D)J transcripts cell by cell. The assembled contigs for each cell were then annotated by mapping the assembled contigs to the Ensemble v87 V(D)J reference sequences. Clonotypes were defined as alpha, beta chain pairs containing unique CDR3 sequences. Clonotypes were filtered for single alpha and single beta chain pairs present at frequency above 2 cells to yield the final list of clonotypes per target peptide in a specific donor.

### Results

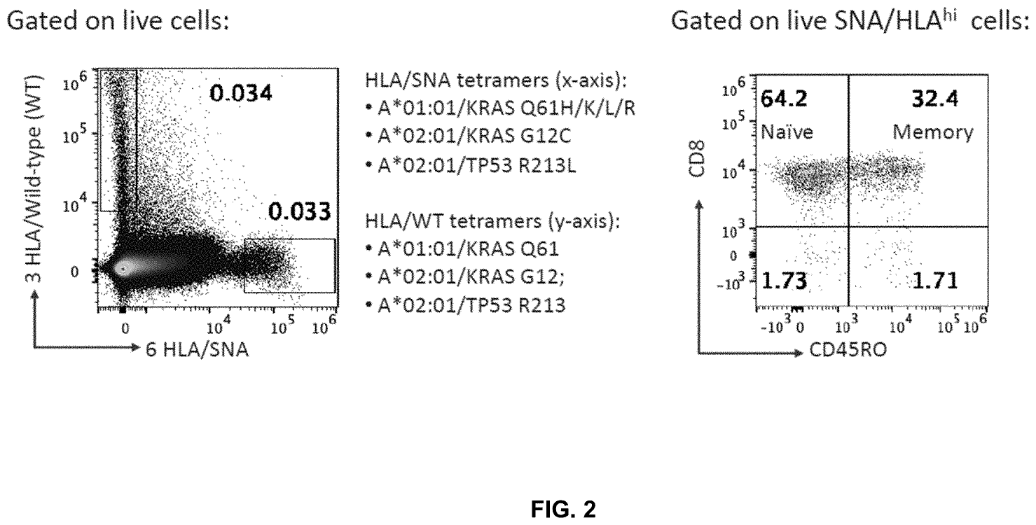

Isolation of neoantigen specific T cells from healthy donors (i.e., donors generally considered in good health with no history of tumor) was assessed. A Pan T Cell Isolation Kit was used to enrich for naïve and memory CD4 and CD8 T cells. As shown in FIG. 2, enriched naïve and memory T cells were effectively labeled using a pool of 6 neoantigen-MHC tetramers to identify neoantigen specific T cells (FIG. 2, left panel, X-axis). The neoantigen-MHC tetramer pool contained A*01:01/KRAS Q61H/K/L/R, A*02:01/KRAS G12C, and A*02:01/TP53 R213L. The labeling also demonstrated effective separation of neoantigen specific T cells from T cells specific for the corresponding wildtype peptide (wildtype specific T cells FIG. 2 left panel, Y-axis), where the wildtype peptide-MHC tetramers were A*01:01/KRAS Q61, A*02:01/KRAS G12; and A*02:01/TP53 R213. Gating on neoantigen-MHC tetramer^{h} (“SNA/HLA^{hi}”) cells also demonstrated that about two-thirds of the neoantigen specific T cells from healthy donors were naïve, while a third demonstrated a memory T cell phenotype (64.2% CD45RO⁻ versus 32.4% CD45RO*; FIG. 2 right panel), indicating that memory T cells (CD45RA-CD45RO+) can be a source of neoantigen-specific TCRs even from a healthy donor who has no history of KRAS or TP53 mutation.

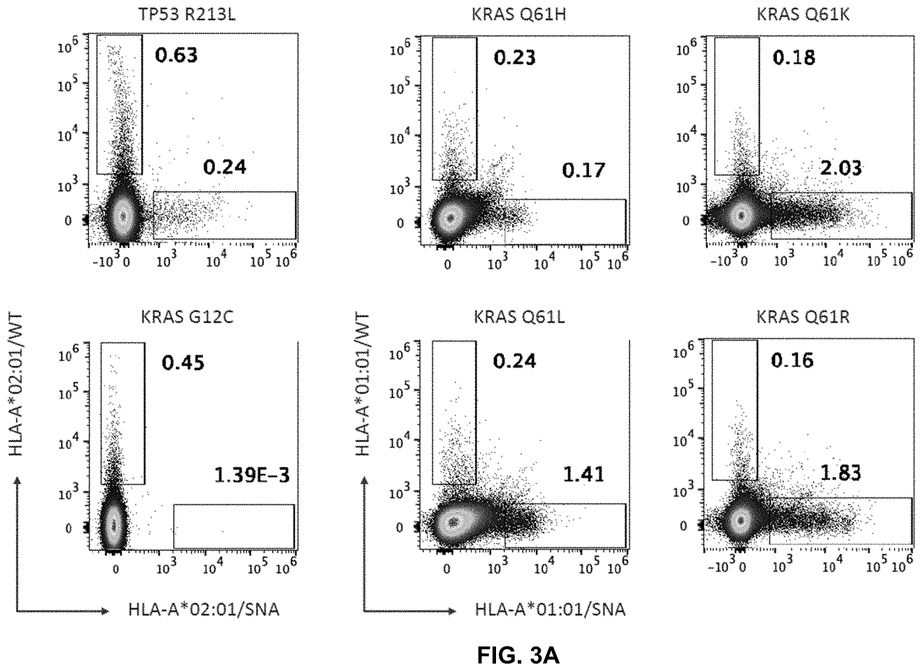

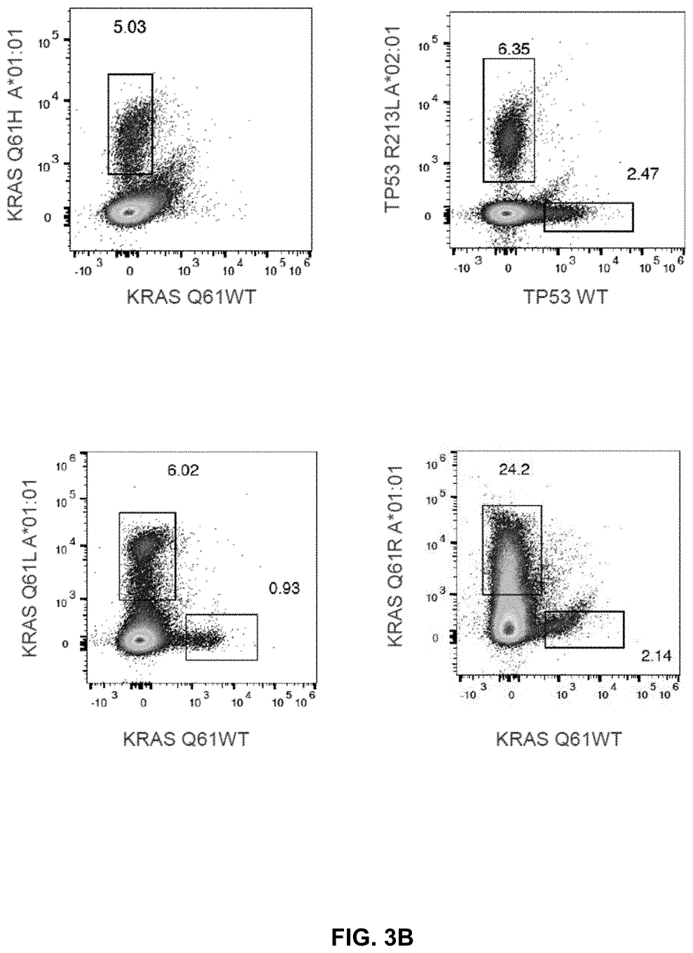

Two weeks after the initial round of pooled sorting/isolation and T cell expansion, cells were divided and individually labeled with each of the 6 neoantigen-MHC tetramers. As shown in FIG. 3A, the expanded cells demonstrated the presence of at least 5 out of the 6 neoantigen specific T cells (A*01:01/KRAS Q61K/L/R/H and A*02:01/TP53 R213L), although the neoantigen specific T cells represented a generally small portion of the total CD8 population (2% or less). To increase frequency of the neoantigen-specific T cells, sorted/isolated peptide-MHC positive cells were expanded an additional week. As shown in FIG. 3B, the expanded cells demonstrated the presence of neoantigen specific T cells (A*01:01/KRAS Q61L/R/H and A*02:01/TP53 R213L), with the neoantigen specific T cells representing between about 5-24% of the total CD8 population. The labeled cell populations were resorted and subsequently processed to for TCR sequencing at single cell level, as described above.

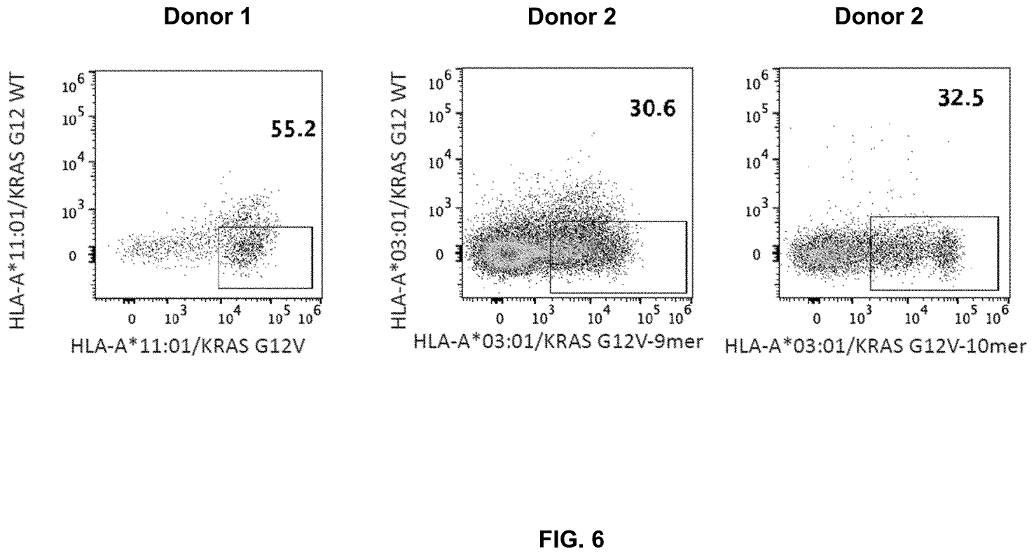

In addition, isolation of neoantigen specific T cells from a naïve population of T cells isolated from healthy donors was also assessed using labeling with a single neoantigen-MHC tetramer. A Naïve CD8 T Cell Isolation Kit & CD4 depletion kit were used to enrich for naïve CD8 T cells. The neoantigen-MHC tetramers used were A*11:01/KRAS G12V, A*03:01/KRAS G12V-9mer; and A*03:01/KRAS G12V-10mer. As shown in FIG. 6, two weeks after the initial round of sorting/isolation and T cell expansion using a single neoantigen-MHC tetramer, relabeling of the expanded cells demonstrated a large population (30-55% of the total CD8 T cell population) of neoantigen specific T cells.

The results of the sorting/isolation experiments demonstrate that while a pooled sorting/isolation method using a mixture of neoantigen-MHC tetramers can be used to isolate neoantigen specific T cells, sorting/isolation using a single neoantigen-MHC tetramer can result in a higher frequency of neoantigen specific T cells.

Next, the TCR sequencing was assessed for the various cell populations isolated as described above. Cells identified using the CTNNB1_S45P tetramer HLA-A*03:01/TTAPPLSGK were also processed. The neoantigen-tetramer labeled expanded cell populations were resorted and subsequently processed for TCR sequencing at single cell level, as described above. As shown in Tables 1A. 1-1A. 3 and Tables 1C. 1-1C. 3, multiple TCR sequences were identified for HLA-A*02:01/KLVVVGACGV, HLA-A*03:01/TTAPPLSGK, HLA-A*03:01/VVGAVGVGK, HLA-A*03:01/VVVGAVGVGK, and HLA-A*11:01/VVGAVGVGK. The results demonstrate that neoantigen-specific TCRs can be identified in a naïve population of T cells from healthy donor cells, including to TCRs specific for different epitopes and/or different HLAs.

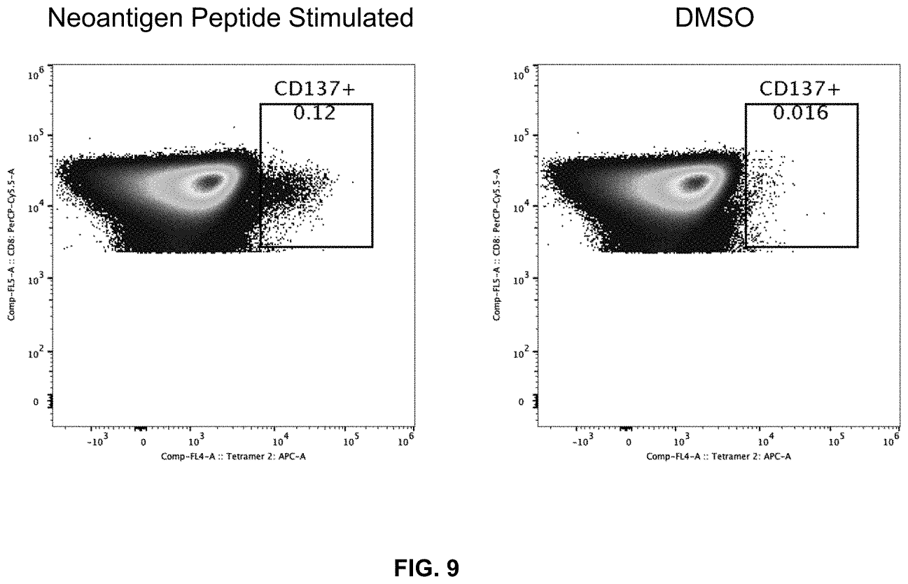

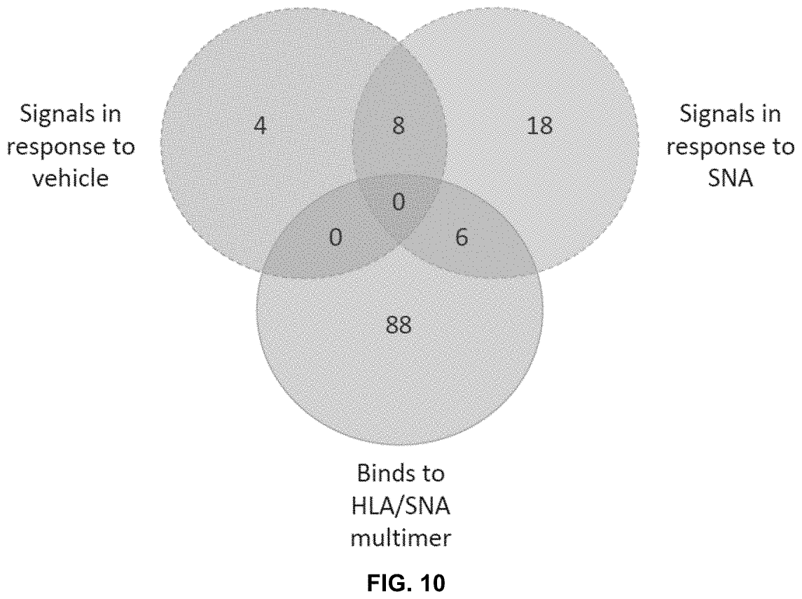

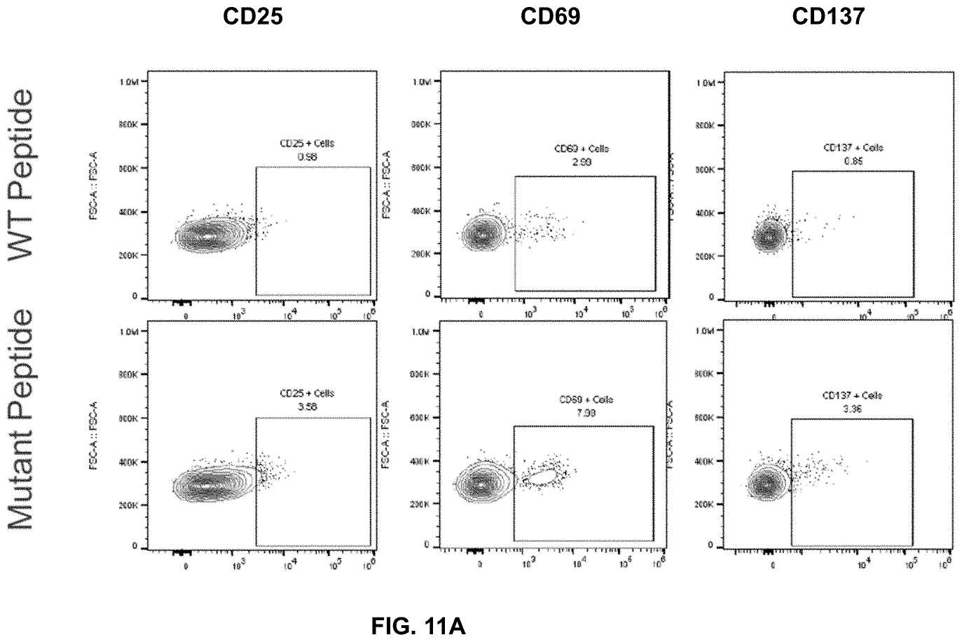

In addition to neoantigen-tetramer labeling of T cells for resorting, cells were resorted based on functional signaling to a cognate peptide. Expanded naïve CD8 T cells that were originally enriched using the single neoantigen-MHC tetramers A*11:01/KRAS G12V were stimulated using PBMCs loaded with the VVGAVGVGK peptide or with DMSO (non-specific signaling control). Functional signaling was determined using CD137 upregulation as a marker. As shown in FIG. 9, after a day of stimulation, cells were gated on CD137+ for neoantigen (FIG. 9 left panel) and DMSO (FIG. 9 right panel) stimulated cells and resorted then sequenced for TCRs, as described above. The TCR sequences determined for (i) neoantigen-tetramer labeled cells; (ii) CD137+ neoantigen-stimulated cells; and (iii) CD137+ DMSO-stimulated cells were compared in silico for shared TCR sequences. A summary of the results is presented in FIG. 10. Specific tetramer binding determined TCR sequences for 94 clonotypes, and of those, 6 TCR sequences were shared with those cells that demonstrated peptide-specific functional signaling. The results demonstrate that functional neoantigen-specific TCRs were identified.

### Example 7: Additional Identification of TCRs that Bind HLA-PEPTIDE Target Neoantigens

Antigen-specific TCRs are identified using the methods described herein, including identification of neoantigen-specific TCRs. For example, TCRs specific for any of SEQ ID NOs:10,755 to 29,364 bound to their cognate HLA allele. The general workflow for identifying antigen-specific TCRs is below:

1) T cells are isolated from HLA-matched healthy donor using magnetic-activated cell sorting (MACS) using: (i) Pan T Cell Isolation Kit to enrich for naïve and memory CD4 and CD8 T cells; (ii) Naïve Pan T Cell Isolation Kit to enrich for naïve T cells; (iii) Naïve CD8 T Cell Isolation Kit & CD4 depletion kit to enrich for naïve CD8 T cells; or (iv) CD8 T Cell Isolation Kit & CD4 depletion kit to enrich for naïve and memory CD8 T cells

a) Alternatively, the source of antigen-specific T cells, may include:
i) healthy donor's memory T cells
ii) single positive CD4 T cells as well as CD4/CD8 double positive T cells
iii) patient-derived, tumor infiltrating lymphocytes (TILs) processed from commercial dissociated tumor cells (DTCs)
iv) patient-derived PBMC, such as patients vaccinated with an antigen/neoantigen of interest
v) T cells are not limited to those harboring conventional αβ heterodimeric TCRs, but can include those with rare TCR configurations such as homodimers (e.g., ββ), heterodimers (e.g., γδ), trimers (βββ), and other combinations of TCR chains

2) peptide-MHC multimers are generated either by using prefolded monomers or commercially available monomers (e.g., Flex-T monomers—BioLegend)

3) Peptide-MHC multimers binding T cells are sorted using fluorescence-activated cell sorting (FACS) method.

4) Sorted T cells are polyclonally expanded with feeder cells and interleukins (IL2 and/or combination of IL7/IL15) for 2-3 weeks. Expansion may also be done in an antigen-specific manner using primary (whole PBMC, DCs, B cells, monocytes) and/or artificial antigen presenting cells (K562, T2, etc.).

5) Post expansion, the resulting cells are exposed to cognate peptide-MHC multimers, and multimer binding cells are sorted (also called re-sort).

6) Re-sorted T cells are sequenced at single cell level (e.g., using 10× Genomics systems, as described above) to obtain TCR sequences containing αβ heterodimeric TCRs or rare TCR configurations such as homodimers (e.g., ββ), heterodimers (e.g., γδ), trimers (ααβ), and other combinations of TCR chains.

a) Expanded T cells may also be divided into 2 or more populations e.g., populations of the cells are:
i) sequenced at single cell level to obtain TCR sequences.
ii) stimulated with physiological concentration of peptide and autologous APCs (PBMCs, B cells, monocytes, DCs). Captured functionally responding cells, e.g. those that secrete cytokines as an example but not limited to IFNg, TNF alpha or IL-2 are sequenced at single cell level to obtain TCR sequences and profile upregulation of activation marker mRNA transcripts.
iii) alternatively, or in addition to cytokines, expression of activation markers (e.g. CD137, CD69 or others) could be used as a sign of stimulated T cell functional response. Selected functional cells are sequenced at single cell level (e.g., using 10× Genomics systems, as described above) to obtain TCR sequences and profile upregulation of activation marker mRNA transcripts

b) Identified TCR sequences undergo quality control steps to identify high-quality and/or specific candidates, with criteria including some or all of the criteria below:
i) excluding sequences with multiple and/or missing TRA or TRB chains;
ii) excluding sequences with internal stop codons;
iii) excluding sequences with TRA or TRB chains less than 90 amino acids in length;
iv) excluding double counting sequences associated with biological and/or technical replicates (i.e., only include sequence once)
v) annotating sequences with a CDR3 of a known epitope (e.g., known CDR3s found in a CDR3 database, such as VDJdb);
vi) excluding sequences associated with a bystander TCR (e.g., TCRs associated with T cells that are non-specific, such as activated by DMSO pulsed APCs).

### Example 8: Screening and Validation of TCRs that Bind HLA-PEPTIDE Target Neoantigens

### Methods

Candidate TCR sequences for screening were identified from healthy donors, as described above. Briefly, TCR clonotypes existing in a multimer-binding population and/or in an activated T cell population meeting the criteria for screening were selected. Criteria include excluding from the candidate library: sequences with multiple and/or missing TRA or TRB chains; sequences with internal stop codons; sequences with TRA or TRB chains less than 90 amino acids in length; sequences associated with a bystander TCR. Additionally, sequences with a CDR3 annotated to be associated a known epitope were not screened.

Lentiviral transduction: For screening assays, a CD8+ Jurkat KO (endogenous TCR knock-out) cell line was transduced with lentivirus to express antigen-specific TCRs. The HIV-derived lentivirus transfer vector was obtained from SBI Biosciences and modified to remove the EFlα promoter and introduce an MSCV promoter followed by a multiple cloning site (MCS) and the TCR constant alpha sequence. Lentivirus support plasmids expressing VSV-G (pCMV-VsvG), Rev (pRSV-Rev) and Gag-pol (pCgpV) were used to produce virus (ViraPower Lentiviral Packaging Mix; ThermoFisher). Lentivirus was prepared by transfection of 80% confluent 10 cm² plates of HEK293 cells with Lipofectamine 2000 (Thermo Fisher), using 36 μl of lipofectamine and 3 pg of the TCR containing plasmid (confirmed by Sanger sequencing) and 9 pg of ViraPower Lentiviral Packaging mix. 10 mL of the virus-containing media were harvested after 48 hours, filtered and concentrated using the Lenti-X system (Clontech), and the virus was resuspended in 100-200 μl of fresh medium. Following viral titering using qPCR, concentrated viral supernatant was added to Jurkat cells. Cells were spun at 1500×g for 45 minutes with 8 pg/mL polybrene at a density of 8×10⁵ cells/mL. Following spinfection, media was added to bring the cell density to 4×10⁵ cells/mL with a final concentration of 4 μg/mL polybrene. Cells were incubated overnight, and the media was completely refreshed after 16 hours. After 72 hours, TCR expression was assessed and, if needed, cells were sorted to obtain high TCR-expression populations. For validation assays, primary T cells from healthy donors were transduced with lentivirus to express antigen-specific TCRs.

Signaling assays: antigen presenting cells K562 cells, constitutively expressing HLA-A*02:01 or HLA-A*11:01, as indicated, were loaded with the indicated mutant or wildtype peptide at 10 μM, or the indicated concentration for antigen titration experiments, for 1 hour. Transduced Jurkat cells or primary T cells were co-cultured overnight (˜20 hours) with the peptide-loaded APCs at a 1:1 ratio of 75,000 TCR-expressing Jurkat cells to 75,000 APCs or a 1:4 ratio of 50,000 primary T cells to 200,000 APCs per well in 96-well plates. After co-culture, the T cell activation markers CD25, CD69, CD137 were measured using flow cytometry (antibodies from BioLegend) and IL-2 cytokine production assessed by MSD (Meso Scale Diagnostics V-PLEX Human IL-2 Kit). For proliferation assays, transduced primary T cells were labeled with CellTrace Violet dye (ThermoFisher) prior to incubation with peptide-loaded APCs. After co-culture, dilution of the CellTrace Violet dye was assessed using flow cytometry to determine proliferation.

### Results

Candidate TCR sequences were identified from healthy donors and selected for screening. Candidates for screening included those sequences shown in Table 1A. 2 and Table 1A. 3.

For screening, CD8+ Jurkat KO (endogenous TCR knock-out) cells were transduced with the candidate TCR sequences. Signaling assays using a candidate TCR's cognate neoantigen peptide or corresponding wildtype peptide were performed to assess specificity and functionality. In addition, signaling was assessed for TCRs that do not recognize a cognate peptide (“negative TCRs”) and MART-1/Melan-A specific TCR DMF5 (MART-1/DMF5 TCR described in detail in Johnson et al. “Gene Transfer of Tumor-Reactive TCR Confers Both High Avidity and Tumor Reactivity to Nonreactive Peripheral Blood Mononuclear Cells and Tumor-Infiltrating Lymphocytes” J Immunol 2006 Nov. 1; 177(9):6548-59, herein incorporated by reference for all purposes). As shown in Table 9, activation markers and cytokine production were noticeably increased when stimulated with cognate RAS G12C and G12V neoantigens in comparison to stimulation with corresponding wildtype peptides. Notably, signaling for several candidate TCRs were comparable to the well-established MART-1/DMF5 control (fold change peptide vs DMSO vehicle only) and demonstrably better than negative TCR signaling. Accordingly, the data demonstrate that functional and specific TCR candidates were identified through screening TCR sequences isolated from healthy donors.

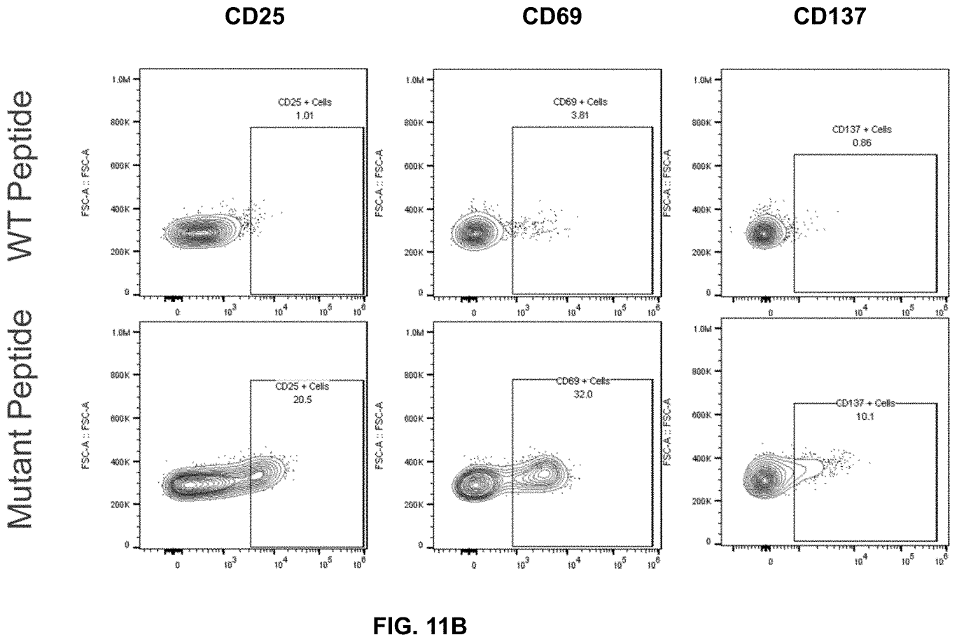

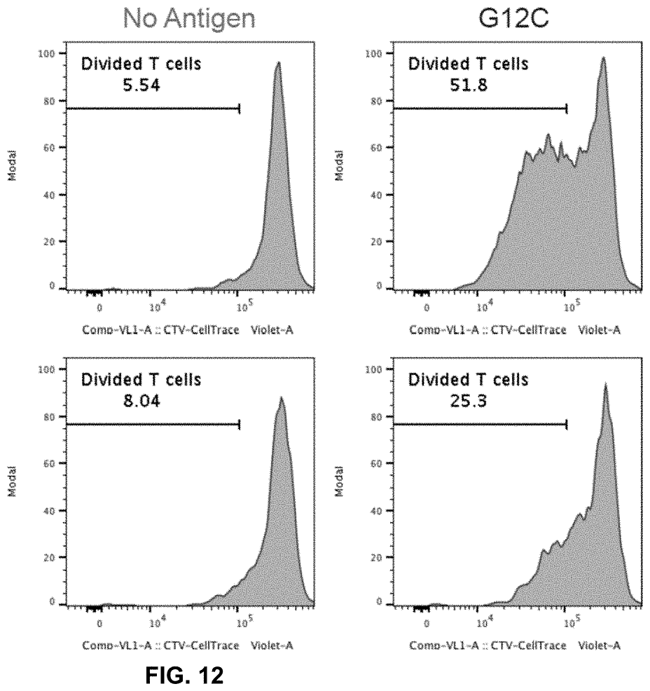

Following initial screening in Jurkat cells, TCR candidates that demonstrated functional and specific signaling were further validated in primary T cells. As shown in Table 9, activation markers in primary T cells were noticeably increased when stimulated with cognate RAS G12C and G12V neoantigens in comparison to stimulation with corresponding wildtype peptides. Notably, signaling for candidate TCRs assayed were demonstrably better than negative TCR signaling. Representative flow cytometry assessments are shown in FIG. 11A (clone 01CA019_064_F05_0005) and FIG. 11B (01CA019_064_F05_0047). Proliferation in primary T cells was also assessed for two of the TCR candidates. As shown in FIG. 12, clones 01CA019_064_F05_0047 and 01CA019_064_F05_0005 demonstrated proliferation in response to neoantigen stimulation relative to stimulation without antigen.

TABLE 9
Screening and Validation Signaling Assay Summary for TCR Candidates

| | | | | |
|---|---|---|---|---|
| | CD25 Fold | CD69 Fold | IL2 Fold | Primary T Cell |
| TCR ID | Change | Change | Change | (CD25/69/137) |

HLA-A*0201 MART-1 TCRs (Control)

| | | | |
|---|---|---|---|
| DMF5 | 235x | 45x | 33x |

HLA-A*0201/KLVVVGACGV G12C TCRs

| | | | | |
|---|---|---|---|---|
| 01CA019_064_F05_0005 | 168x | 27x | 30x | 5x, 8x, 3x |
| “TCR66” | | | | |
| 0ICAO19_064_F05_0047 | 25x | 17x | 11X | 4x, 10x, 3x |
| “TCR61” | | | | |
| 01CA019_064_F05_0104 | 15x | 38x | 5x | |
| “TCR10” | | | | |

HLA-A*1101/VVGAVGVGK G12V TCRs

| | | | | |
|---|---|---|---|---|
| 01CA018_064_F10_0022 | 161x | 51x | 26x | 4.5x, 6x, 3x |
| “TCR27” | | | | |
| 01CA018_064_F10_0001 | 107x | 31x | 65x | 2.5x, 4.5x, 3x |
| “TCR56” | | | | |

Negative TCRs (Control)

| | | | | |
|---|---|---|---|---|
| Negative TCR | 0.7x | 1.2x | 0.2x | 1x, 1x, 1.2x |

*Fold-change is calculated a difference in signal of cognate neoantigen peptide reative to corresponding wildtype peptide

### Sequence Tables

TABLE A

| | | | HLA | Restricted |
|---|---|---|---|---|
| SEQ | | | Class 1 | Peptide amino |
| ID NO: | Gene | Mutation | subtype | acid sequence |
| 29358 | CTNNB1 | S37Y point | HLA-A* | YLDSGIHYGA |
| | | mutation | 02:01 | |
| 29359 | CHD4 | CHD4_K73fs | HLA-B* | TVRAATIL |
| | | | 08:01 | |
| 29360 | CTNNB1 | CTNNB_1S45P | A*11:01 | TTAPPLSGK |
| 29361 | CTNNB1 | CTNNB1_T41A | A*11:01 | ATAPSLSGK |
| 29362 | RAS | RAS_G12V | A*03:01 | VVGAVGVGK |
| 29363 | KRAS/ | KRAS/ | A*01:01 | ILDTAGREEY |
| | NRAS | NRAS_Q61R | | |
| 29364 | TP53 | TP53_R213L | A*02:01 | YLDDRNTFL |

Refer to Sequence Listing, SEQ ID NOS. 10,755-21,015.

Table A includes HLA-PEPTIDE neoantigens, wherein a specific restricted peptide having a specific amino acid sequence is predicted to associate with a given HLA allele with an EDGE score >0.001. The restricted peptide corresponds to a peptide fragment containing a somatic mutation associated with a cancer.

For clarity, each HLA-PEPTIDE neoantigen in Table A is assigned a unique SEQ ID NO. Each of the above sequence identifiers is associated with the Table identifier (i.e., Table A), the HLA Class I subtype, the gene name corresponding to the restricted peptide, the somatic mutation, whether the prevalence of the peptide:HLA pair was 0.1% or greater (noted as “1”) or less than 0.1% (noted as “0”), and the amino acid sequence of the restricted peptide. For example, the HLA-PEPTIDE neoantigen designated as SEQ ID NO: 10755 is a CREB3L1 V414I neoantigen that is HLA-PEPTIDE target HLA-A*02:06_AADGIYTA. As indicated by SEQ ID NO: 10755, the restricted peptide AADGIYTA contains the V414I mutation in the protein encoded by gene CREB3L1, and the HLA-PEPTIDE target has a prevalence less than 0.1%.

Table A HLA-PEPTIDE neoantigens are disclosed in PCT/US2019/033830, filed on May 23, 2019, which application is hereby incorporated by reference in its entirety.

AACR GENIE Results

Refer to Sequence Listing, SEQ ID NOS. 21,016-29,357.

AACR GENIE results includes HLA-PEPTIDE neoantigens wherein a specific restricted peptide having a specific amino acid sequence is predicted to associate with a given HLA allele with an EDGE score >0.001 and prevalence >0.10%. The restricted peptide corresponds to a peptide fragment containing a somatic mutation associated with a cancer.

For clarity, each HLA-PEPTIDE neoantigen in the AACR GENIE results is assigned a unique SEQ ID. NO. Each of the above sequence identifiers includes a designation as an AACR GENIE result, the gene name corresponding to the restricted peptide, the type and nature of somatic mutation, the HLA Class I subtype, and the amino acid sequence of the restricted peptide. For the AACR GENIE results, the HLA Class I subtype designation is expressed as a single letter followed by a 4-digit code.

For clarity, the designation “p.” indicates a change in the protein sequence, the designation “fs*number” stands for a frameshift mutation causing a stop codon in [the designated number] of amino acids, the designation “dup” stands for an in-frame sequence insertion of a sequence flanked by the designated amino acids, and the designation “del” stands for an in-frame sequence deletion of the designated amino acids.

For example, the HLA-PEPTIDE neoantigen designated as SEQ ID NO: 21016 is a ACVR1 neoantigen carrying point mutation S290L (denoted as “ACVR1_p.S290L”) that is HLA-PEPTIDE target HLA-A*29:02_HYHEMGLLY. As indicated by SEQ ID NO: 21016, the restricted peptide HYHEMGLLY contains the S290L point mutation in the protein encoded by gene ACVR1.

For example, the HLA-PEPTIDE neoantigen designated as SEQ ID NO: 25566 is an NF1 neoantigen carrying insertion or deletion mutation Y2285Tfs*5 (denoted as “NF1_p.Y2285Tfs*5”) resulting in an HLA-PEPTIDE target HLA-A*11:01_KGPDTTVKF. As indicated by SEQ ID NO: 25566, the restricted peptide KGPDTTVKF contains the substitution Y2285T and subsequent sequence that is frameshifted from the normal reading frame of the NF1 gene, resulting in a stop codon in 5 amino acids.

For example, the HLA-PEPTIDE neoantigen designated as SEQ ID NO: 22713 is a CDKN2A neoantigen carrying an in-frame sequence insertion T18_A19dup (denoted as “CDKN2A_p.T18_A19dup”), resulting in the HLA-PEPTIDE target HLA-A*68:01_ATATAAARGR. As indicated by SEQ ID NO: 22713, the restricted peptide ATATAAARGR contains an insertion of amino acids T and A at amino acid positions 18 and 19, and its surrounding sequence in the CDKN2A protein.

For example, the HLA-PEPTIDE neoantigen designated as SEQ ID NO: 23233 is a CTNNB1 neoantigen carrying an in-frame sequence deletion S45del (denoted as “CTNNB1_p.S45del”), resulting in and HLA-PEPTIDE target HLA-A*03:01_TTTAPLSGK. As indicated by SEQ ID NO: 23233, the restricted peptide TTTAPLSGK includes the deletion S45del and its surrounding sequence in the CTNNB1 gene.

SEQ ID NOs 29358-29364

TABLE 1A.1
Alpha VJ and beta V(D)J sequences for TCRs isolated
from healthy donors

| Clone ID | Alpha | Beta |
|---|---|---|
| 01CA0WT_066_ | MEKNPLAAPLLILWFHLDCVSSILNV | MSNQVLCCVVLCFLGANTVDGGITQS |
| F03_0008 | EQSPQSLHVQEGDSTNFTCSFPSSNF | PKYLFRKEGQNVTLSCEQNLNHDAMY |
| | YALHWYRWETAKSPEALFVMTLNG | WYRQDPGQGLRLIYYSQIVNDFQKGD |
| | DEKKKGRISATLNTKEGYSYLYIKGS | IAEGYSVSREKKESFPLTVTSAQKNPT |
| | QPEDSATYLCAFPMDSNYQLIWGAG | AFYLCASSLGVHGYTFGSGTRLTVV |
| | TKLIIKP | |
| 01CA019_064_ | MWGVFLLYVSMKMGGTTGQNIDQP | MGFRLLCCVAFCLLGAGPVDSGVTQT |
| F05_1114 | TEMTATEGAIVQINCTYQTSGFNGLF | PKHLITATGQRVTLRCSPRSGDLSVYW |
| | WYQQHAGEAPTFLSYNVLDGLEEK | YQQSLDQGLQFLIQYYNGEERAKGNIL |
| | GRFSSFLSRSKGYSYLLLKELQMKD | ERFSAQQFPDLHSELNLSSLELGDSAL |
| | SASYLCAVSSGNTGKLIFGQGTTLQ | YFCASSVAGDSLTDTQYFGPGTRLTVL |
| | VKPD | |
| 01CA0DP_064_ | MACPGFLWALVISTCLEFSMAQTVT | MGTSLLCWVVLGFLGTDHTGAGVSQS |
| F04_0003 | QSQPEMSVQEAETVTLSCTYDTSES | PRYKVTKRGQDVALRCDPISGHVSLY |
| | DYYLFWYKQPPSRQMILVIRQEAYK | WYRQALGQGPEFLTYFNYEAQQDKSG |
| | QQNATENRFSVNFQKAAKSFSLKIS | LPNDRFSAERPEGSISTLTIQRTEQRD |
| | DSQLGDAAMYFCAPITGAGSYQLTF | SAMYRCASSLAFQESNTGELFFGEGSR |
| | GKGTKLSVIP | LTVL |
| 01DB008_064_ | MKSLRVLLVILWLQLSWVWSQQKE | MGTRLFFYVALCLLWAGHRDAGITQS |
| G02_0013 | VEQNSGPLSVPEGAIASLNCTYSDRG | PRYKITETGRQVTLMCHQTWSHSYMF |
| | SQSFFWYRQYSGKSPELIMFIYSNGD | WYRQDLGHGLRLIYYSAAADITDKGE |
| | KEDGRFTAQLNKASQYVSLLIRDSQ | VPDGYVVSRSKTENFPLTLESATRSQT |
| | PSDSATYLCAALNSGGYQKVTFGTG | SVYFCASSPGLNEQFFGPGTRLTVL |
| | TKLQVIP | |
| 01DB008_064_ | MKSLRVLLVILWLQLSWVWSQQKE | MGTSLLCWMALCLLGADHADTGVSQ |
| G02_0023 | VEQNSGPLSVPEGAIASLNCTYSDRG | NPRHKITKRGQNVTFRCDPISEHNRLY |
| | SQSFFWYRQYSGKSPELIMFIYSNGD | WYRQTLGQGPEFLTYFQNEAQLEKSR |
| | KEDGRFTAQLNKASQYVSLLIRDSQ | LLSDRFSAERPKGSFSTLEIQRTEQGD |
| | PSDSATYLCAVGTNDMRFGAGTRLT | SAMYLCASTSPPEAFFGQGTRLTVV |
| | VKP | |
| 01CA018_064_ | MWGVFLLYVSMKMGGTTGQNIDQP | MGTSLLCWMALCLLGADHADTGVSQ |
| H10_0018 | TEMTATEGAIVQINCTYQTSGFNGLF | NPRHKITKRGQNVTFRCDPISEHNRLY |
| | WYQQHAGEAPTFLSYNVLDGLEEK | WYRQTLGQGPEFLTYFQNEAQLEKSR |
| | GRFSSFLSRSKGYSYLLLKELQMKD | LLSDRFSAERPKGSFSTLEIQRTEQGD |
| | SASYLCAVRGSNDYKLSFGAGTTVT | SAMYLCASSSDNVKNYGYTFGSGTRLT |
| | VRA | W |
| 01CA018_064_ | MKSLRVLLVILWLQLSWVWSQQKE | MGFRLLCCVAFCLLGAGPVDSGVTQT |
| H10_0014 | VEQNSGPLSVPEGAIASLNCTYSDRG | PKHLITATGQRVTLRCSPRSGDLSVYW |
| | SQSFFWYRQYSGKSPELIMFIYSNGD | YQQSLDQGLQFLIQYYNGEERAKGNIL |
| | KEDGRFTAQLNKASQYVSLLIRDSQ | ERFSAQQFPDLHSELNLSSLELGDSAL |
| | PSDSATYLCAVRNNNDMRFGAGTR | YFCASSVGSDTQYFGPGTRLTVL |
| | LTVKP | |
| 01CA018_022_ | MWGAFLLYVSMKMGGTAGQSLEQ | MLLLLLLLGPGSGLGAVVSQHPSWVI |
| E04_0155 | PSEVTAVEGAIVQINCTYQTSGFYGL | CKSGTSVKIECRSLDFQATTMFWYRQ |
| | SWYQQHDGGAPTFLSYNALDGLEE | FPKKSLMLMATSNEGSKATYEQGVEK |
| | TGRFSSFLSRSDSYGYLLLQELQMK | DKFLINHASLTLSTLTVTSAHPEDSSF |
| | DSASYFCAGFTSGTYKYIFGTGTRLK | YICSARVSFGVMNTEAFFGQGTRLTVV |
| | VLA | |
| 01CA018_022_ | MMKSLRVLLVILWLQLSWVWSQQK | MASLLFFCGAFYLLGTGSMDADVTQT |
| E04_0030 | EVEQDPGPLSVPEGAIVSLNCTYSNS | PRNRITKTGKRIMLECSQTKGHDRMY |
| | AFQYFMWYRQYSRKGPELLMYTYS | WYRQDPGLGLRLIYYSFDVKDINKGEI |
| | SGNKEDGRFTAQVDKSSKYISLFIRD | SDGYSVSRQAQAKFSLSLESAIPNQTA |
| | SQPSDSATYLCASYNNNDMRFGAGT | LYFCATSGRTGGNLETQYFGPGTRLLV |
| | RLTVKP | L |
| 01CADMS_064_ | METLLGLLILWLQLQWVSSKQEVTQ | MLLLLLLLGPGSGLGAVVSQHPSWVI |
| F09_0013 | IPAALSVPEGENLVLNCSFTDSAIYN | CKSGTSVKIECRSLDFQATTMFWYRQ |
| | LQWFRQDPGKGLTSLLLIQSSQREQT | FPKQSLMLMATSNEGSKATYEQGVEK |
| | SGRLNASLDKSSGRSTLYIAASQPGD | DKFLINHASLTLSTLTVTSAHPEDSSF |
| | SATYLCASSNYGGSQGNLIFGKGTK | YICSARAAAAYEQYFGPGTRLTVT |
| | LSVKP | |
| 01CA0WT_064_ | MLLLLVPAFQVIFTLGGTRAQSVTQ | MSLGLLCCGAFSLLWAGPVNAGVTQT |
| F06_0020 | LDSQVPVFEEAPVELRCNYSSSVSVY | PKFRVLKTGQSMTLLCAQDMNHEYM |
| | LFWYVQYPNQGLQLLLKYLSGSTLV | YWYRQDPGMGLRLIHYSVGEGTTAK |
| | KGINGFEAEFNKSQTSFHLRKPSVHI | GEVPDGYNVSRLKKQNFLLGLESAAP |
| | SDTAEYFCAVSEMGGTSYGKLTFGQ | SQTSVYFCASSRTGTAYQPQHFGDGT |
| | GTILTVHP | RLSIL |
| 01CA018_064_ | MWGVFLLYVSMKMGGTTGQNIDQP | MGSRLLCWVLLCLLGAGPVKAGVTQ |
| G01_0167 | TEMTATEGAIVQINCTYQTSGFNGLF | TPRYLIKTRGQQVTLSCSPISGHRSVS |
| | WYQQHAGEAPTFLSYNVLDGLEEK | WYQQTPGQGLQFLFEYFSETQRNKGN |
| | GRFSSFLSRSKGYSYLLLKELQMKD | FPGRFSGRQFSNSRSEMNVSTLELGDS |
| | SASYLCAVDTGFQKLVFGTGTRLLV | ALYLCASSLGSPEAFFGQGTRLTVV |
| | SP | |

TABLE 1A.2
Alpha VJC and beta V(D)JC sequences for TCRs isolated from healthy
donors-G12C/HLA-A*0201

| | Alpha | Beta |
|---|---|---|
| Clone ID | (Constant Region Bolded) | (Constant Region Bolded) |
| 01CA019_064_ | MKTFAGFSFLFLWLQLDCMSRGED | MLLLLLLLGPGSGLGAVVSQHPSWVI |
| F05_0104 | VEQSLFLSVREGDSSVINCTYTDSSS | CKSGTSVKIECRSLDFQATTMFWYRQ |
| | TYLYWYKQEPGAGLQLLTYIFSNMD | FPKQSLMLMATSNEGSKATYEQGVEK |
| | MKQDQRLTVLLNKKDKHLSLRIADT | DKFLINHASLTLSTLTVTSAHPEDSSFY |
| | QTGDSAIYFCAERVNTDKLIFGTGTR | ICSASTGDSGNTIYFGEGSWLTVVEDL |
| | LQVFPNIQNPDPAVYQLRDSKSSDK | NKVFPPEVAVFEPSEAEISHTQKATL |
| | SVCLFTDFDSQTNVSQSKDSDVYIT | VCLATGFFPDHVELSWWVNGKEVH |
| | DKCVLDMRSMDFKSNSAVAWSNK | SGVCTDPQPLKEQPALNDSRYCLSS |
| | SDFACANAFNNSIIPEDTFFPSPESS | RLRVSATFWQNPRNHFRCQVQFYG |
| | CDVKLVEKSFETDTNLNFQNLSVI | LSENDEWTQDRAKPVTQIVSAEAW |
| | GFRILLLKVAGFNLLMTLRLWSS | GRADCGFTSVSYQQGVLSATILYEIL |
| | | LGKATLYAVLVSALVLMAMVKRKDF |
| 01CA019_064_ | MSLSSLLKVVTASLWLGPGIAQKITQ | MGFRLLCCVAFCLLGAGPVDSGVTQT |
| F05_0047 | TQPGMFVQEKEAVTLDCTYDTSDQS | PKHLITATGQRVTLRCSPRSGDLSVYW |
| | YGLFWYKQPSSGEMIFLIYQGSYDE | YQQ SLDQGLQFLIQYYNGEERAKGNIL |
| | QNATEGRYSLNFQKARKSANLVISA | ERFSAQQFPDLHSELNLSSLELGDSAL |
| | SQLGDSAMYFCAMRELNTDKLIFGT | YFCASSQWDSSGNTIYFGEGSWLTVV |
| | GTRLQVFPNIQNPDPAVYQLRDSKS | EDLNKVFPPEVAVFEPSEAEISHTQK |
| | SDKSVCLFTDFDSQTNVSQSKDSD | ATLVCLATGFFPDHVELSWWVNGK |
| | VYITDKCVLDMRSMDFKSNSAVA | EVHSGVCTDPQPLKEQPALNDSRYC |
| | WSNKSDFACANAFNNSIIPEDTFFP | LSSRLRVSATFWQNPRNHFRCQVQF |
| | SPESSCDVKLVEKSFETDTNLNFQ | YGLSENDEWTQDRAKPVTQIVSAEA |
| | NLSVIGFRILLLKVAGFNLLMTLR | WGRADCGFTSVSYQQGVLSATILYE |
| | LWSS | ILLGKATLYAVLVSALVLMAMVKR |
| | | KDF |
| 01CA019_064_ | MSLSSLLKVVTASLWLGPGIAQKITQ | MGTRLLFWVAFCLLGADHTGAGVSQ |
| F05_0064 | TQPGMFVQEKEAVTLDCTYDTSDQS | SPSNKVTEKGKDVELRCDPISGHTALY |
| | YGLFWYKQPSSGEMIFLIYQGSYDE | WYRQSLGQGLEFLIYFQGNSAPDKSG |
| | QNATEGRYSLNFQKARKSANLVISA | LPSDRFSAERTGGSVSTLTIQRTQQEDS |
| | SQLGDSAMYFCAMREPRSYNTDKLI | AVYLCASSLVSQVGYGYTFGSGTRLT |
| | FGTGTRLQVFPNIQNPDPAVYQLRD | VVEDLNKVFPPEVAVFEPSEAEISHT |
| | SKSSDKSVCLFTDFDSQTNVSQSKD | QKATLVCLATGFFPDHVELSWWVN |
| | SDVYITDKCVLDMRSMDFKSNSAV | GKEVHSGVCTDPQPLKEQPALNDSR |
| | AWSNKSDFACANAFNNSIIPEDTFF | YCLSSRLRVSATFWQNPRNHFRCQV |
| | PSPESSCDVKLVEKSFETDTNLNFQ | QFYGLSENDEWTQDRAKPVTQIVSA |
| | NLSVIGFRILLLKVAGFNLLMTLR | EAWGRADCGFTSVSYQQGVLSATIL |
| | LWSS | YEILLGKATLYAVLVSALVLMAMV |
| | | KRKDF |
| 01CA019_064_ | MSLSSLLKVVTASLWLGPGIAQKITQ | MGFRLLCCVAFCLLGAGPVDSGVTQT |
| F05_0005 | TQPGMFVQEKEAVTLDCTYDTSDQS | PKHLITATGQRVTLRCSPRSGDLSVYW |
| | YGLFWYKQPSSGEMIFLIYQGSYDE | YQQSLDQGLQFLIQYYNGEERAKGNIL |
| | QNATEGRYSLNFQKARKSANLVISA | ERFSAQQFPDLHSELNLSSLELGDSAL |
| | SQLGDSAMYFCAMREESSYKLIFGS | YFCASSVAGDSLTDTQYFGPGTRLTVL |
| | GTRLLVRPDIQNPDPAVYQLRDSKS | EDLKNVFPPKVAVFEPSEAEISHTQK |
| | SDKSVCLFTDFDSQTNVSQSKDSD | ATLVCLATGFYPDHVELSWWVNGK |
| | VYITDKCVLDMRSMDFKSNSAVA | EVHSGVCTDPQPLKEQPALNDSRYC |
| | WSNKSDFACANAFNNSIIPEDTFFP | LSSRLRVSATFWQNPRNHFRCQVQF |
| | SPESSCDVKLVEKSFETDTNLNFQ | YGLSENDEWTQDRAKPVTQIVSAEA |
| | NLSVIGFRILLLKVAGFNLLMTLR | WGRADCGFTSESYQQGVLSATILYE |
| | LWSS | ILLGKATLYAVLVSALVLMAMVKR |
| | | KDSRG |

TABLE 1A.3
Alpha VJC and beta V(D)JC sequences for TCRs isolated from healthy
donors-G12V/HLA-A*1101

| | Alpha | Beta |
|---|---|---|
| Clone ID | (Constant Region Bolded) | (Constant Region Bolded) |
| 01CA018_064_ | MAFWLRRLGLHFRPHLGRRMESFLG | MGPQLLGYVVLCLLGAGPLEAQVTQ |
| F08_0007 | GVLLILWLQVDWVKSQKIEQNSEAL | NPRYLITVTGKKLTVTCSQNMNHEY |
| | NIQEGKTATLTCNYTNYSPAYLQWY | MSWYRQDPGLGLRQIYYSMNVEVTD |
| | RQDPGRGPVFLLLIRENEKEKRKERL | KGDVPEGYKVSRKEKRNFPLILESPSP |
| | KVTFDTTLKQSLFHITASQPADSATYL | NQTSLYFCASSLVGNEQFFGPGTRLT |
| | CALGYSSASKIIFGSGTRLSIRPNIQNP | VLEDLKNVFPPKVAVFEPSEAEISH |
| | DPAVYQLRDSKSSDKSVCLFTDFDS | TQKATLVCLATGFYPDHVELSWW |
| | QTNVSQSKDSDVYITDKCVLDMRS | VNGKEVHSGVCTDPQPLKEQPALN |
| | MDFKSNSAVAWSNKSDFACANAFN | DSRYCLSSRLRVSATFWQNPRNHF |
| | NSIIPEDTFFPSPESSCDVKLVEKSFE | RCQVQFYGLSENDEWTQDRAKPV |
| | TDTNLNFQNLSVIGFRILLLKVAGF | TQIVSAEAWGRADCGFTSESYQQG |
| | NLLMTLRLWSS | VLSATILYEILLGKATLYAVLVSAL |
| | | VLMAMVKRKDSRG |
| 01CA018_064_ | MAMLLGASVLILWLQPDWVNSQQK | MGSRLLCWVLLCLLGAGPVKAGVTQ |
| F10_0022 | NDDQQVKQNSPSLSVQEGRISILNCD | TPRYLIKTRGQQVTLSCSPISGHRSVS |
| | YTNSMFDYFLWYKKYPAEGPTFLISIS | WYQQTPGQGLQFLFEYFSETQRNKG |
| | SIKDKNEDGRFTVFLNKSAKHLSLHI | NFPGRFSGRQFSNSRSEMNVSTLELG |
| | VPSQPGDSAVYFCAANGGFQKLVFG | DSALYLCASSSGRTEAFFGQGTRLTV |
| | TGTRLLVSPNIQNPDPAVYQLRDSKS | VEDLNKVFPPEVAVFEPSEAEISHT |
| | SDKSVCLFTDFDSQTNVSQSKDSDV | QKATLVCLATGFFPDHVELSWWV |
| | YITDKCVLDMRSMDFKSNSAVAWS | NGKEVHSGVCTDPQPLKEQPALND |
| | NKSDFACANAFNNSIIPEDTFFPSPE | SRYCLSSRLRVSATFWQNPRNHFR |
| | SSCDVKLVEKSFETDTNLNFQNLSV | CQVQFYGLSENDEWTQDRAKPVT |
| | IGFRILLLKVAGFNLLMTLRLWSS | QIVSAEAWGRADCGFTSVSYQQGV |
| | | LSATILYEILLGKATLYAVLVSALV |
| | | LMAMVKRKDF |
| 01CA018_064_ | METLLGVSLVILWLQLARVNSQQGE | MGTRLLCWAALCLLGAELTEAGVAQ |
| F10_0004 | EDPQALSIQEGENATMNCSYKTSINN | SPRYKIIEKRQSVAFWCNPISGHATLY |
| | LQWYRQNSGRGLVHLILIRSNEREKH | WYQQILGQGPKLLIQFQNNGVVDDS |
| | SGRLRVTLDTSKKSSSLLITASRAADT | QLPKDRFSAERLKGVDSTLKIQPAKL |
| | ASYFCATDAGTGGFKTIFGAGTRLFV | EDSAVYLCASSLESSYEQYFGPGTRL |
| | KANIQNPDPAVYQLRDSKSSDKSVC | TVTEDLNKVFPPEVAVFEPSEAEISH |
| | LFTDFDSQTNVSQSKDSDVYITDKC | TQKATLVCLATGFFPDHVELSWW |
| | VLDMRSMDFKSNSAVAWSNKSDFA | VNGKEVHSGVCTDPQPLKEQPALN |
| | CANAFNNSIIPEDTFFPSPESSCDVK | DSRYCLSSRLRVSATFWQNPRNHF |
| | LVEKSFETDTNLNFQNLSVIGFRILL | RCQVQFYGLSENDEWTQDRAKPV |
| | LKVAGFNLLMTLRLWSS | TQIVSAEAWGRADCGFTSVSYQQG |
| | | VLSATILYEILLGKATLYAVLVSAL |
| | | VLMAMVKRKDF |
| 01CA018_064_ | METLLGVSLVILWLQLARVNSQQGE | MGTRLLCWAALCLLGAELTEAGVAQ |
| F10_0005 | EDPQALSIQEGENATMNCSYKTSINN | SPRYKIIEKRQSVAFWCNPISGHATLY |
| | LQWYRQNSGRGLVHLILIRSNEREKH | WYQQILGQGPKLLIQFQNNGVVDDS |
| | SGRLRVTLDTSKKSSSLLITASRAADT | QLPKDRFSAERLKGVDSTLKIQPAKL |
| | ASYFCATDRGSSNTGKLIFGQGTTLQ | EDSAVYLCASSLEISYEQYFGPGTRLT |
| | VKPDNIQNPDPAVYQLRDSKSSDKS | VTEDLKNVFPPKVAVFEPSEAEISH |
| | VCLFTDFDSQTNVSQSKDSDVYITD | TQKATLVCLATGFYPDHVELSWW |
| | KCVLDMRSMDFKSNSAVAWSNKSD | VNGKEVHSGVCTDPQPLKEQPALN |
| | FACANAFNNSIIPEDTFFPSPESSCDV | DSRYCLSSRLRVSATFWQNPRNHF |
| | KLVEKSFETDTNLNFQNLSVIGFRIL | RCQVQFYGLSENDEWTQDRAKPV |
| | LLKVAGFNLLMTLRLWSS | TQIVSAEAWGRADCGFTSESYQQG |
| | | VLSATILYEILLGKATLYAVLVSAL |
| | | VLMAMVKRKDSRG |
| 01CA018_064_ | METLLGVSLVILWLQLARVNSQQGE | MGTRLLCWAALCLLGAELTEAGVAQ |
| F10_0002 | EDPQALSIQEGENATMNCSYKTSINN | SPRYKIIEKRQSVAFWCNPISGHATLY |
| | LQWYRQNSGRGLVHLILIRSNEREKH | WYQQILGQGPKLLIQFQNNGVVDDS |
| | SGRLRVTLDTSKKSSSLLITASRAADT | QLPKDRFSAERLKGVDSTLKIQPAKL |
| | ASYFCATDSQTGANNLFFGTGTRLTV | EDSAVYLCASSLGDTYEQYFGPGTRL |
| | IPYIQNPDPAVYQLRDSKSSDKSVCL | TVTEDLKNVFPPKVAVFEPSEAEISH |
| | FTDFDSQTNVSQSKDSDVYITDKCV | TQKATLVCLATGFYPDHVELSWW |
| | LDMRSMDFKSNSAVAWSNKSDFAC | VNGKEVHSGVCTDPQPLKEQPALN |
| | ANAFNNSIIPEDTFFPSPESSCDVKL | DSRYCLSSRLRVSATFWQNPRNHF |
| | VEKSFETDTNLNFQNLSVIGFRILLL | RCQVQFYGLSENDEWTQDRAKPV |
| | KVAGFNLLMTLRLWSS | TQIVSAEAWGRADCGFTSESYQQG |
| | | VLSATILYEILLGKATLYAVLVSAL |
| | | VLMAMVKRKDSRG |
| 01CA018_064_ | METLLGVSLVILWLQLARVNSQQGE | MGTRLLCWAALCLLGAELTEAGVAQ |
| F10_0001 | EDPQALSIQEGENATMNCSYKTSINN | SPRYKIIEKRQSVAFWCNPISGHATLY |
| | LQWYRQNSGRGLVHLILIRSNEREKH | WYQQILGQGPKLLIQFQNNGVVDDS |
| | SGRLRVTLDTSKKSSSLLITASRAADT | QLPKDRFSAERLKGVDSTLKIQPAKL |
| | ASYFCATDPRELSFGAGTTVTVRANI | EDSAVYLCASSLESSYEQYFGPGTRL |
| | QNPDPAVYQLRDSKSSDKSVCLFTD | TVTEDLKNVFPPKVAVFEPSEAEISH |
| | FDSQTNVSQSKDSDVYITDKCVLDM | TQKATLVCLATGFYPDHVELSWW |
| | RSMDFKSNSAVAWSNKSDFACANA | VNGKEVHSGVCTDPQPLKEQPALN |
| | FNNSIIPEDTFFPSPESSCDVKLVEKS | DSRYCLSSRLRVSATFWQNPRNHF |
| | FETDTNLNFQNLSVIGFRILLLKVA | RCQVQFYGLSENDEWTQDRAKPV |
| | GFNLLMTLRLWSS | TQIVSAEAWGRADCGFTSESYQQG |
| | | VLSATILYEILLGKATLYAVLVSAL |
| | | VLMAMVKRKDSRG |

TABLE 1B
Alpha VJ and beta V(D)J sequences for TCRs isolated from TILs
derived from subjects with cancer

| Clone ID | Alpha | Beta |
|---|---|---|
| 01CA018_074_ | MWGVFLLYVSMKMGGTTGQ | MGCRLLCCAVLCLLGAVPMET |
| E09_0061 | NIDQPTEMTATEGAIVQINCTY | GVTQTPRHLVMGMTNKKSLK |
| | QTSGFNGLFWYQQHAGEAPTF | CEQHLGHNAMYWYKQSAKKP |
| | LSYNVLDGLEEKGRFSSFLSRS | LELMFVYSLEERVENNSVPSRF |
| | KGYSYLLLKELQMKDSASYLC | SPECPNSSHLFLHLHTLQPEDS |
| | AVTDSNYQLIWGAGTKLIIKP | ALYLCASSQGTGSGANVLTFG |
| | | AGSRLTVL |
| 01CA018_074_ | METLLGLLILWLQLQWVSSKQ | MLLLLLLLGPGSGLGAVVSQH |
| E09_0035 | EVTQIPAALSVPEGENLVLNCS | PSWVICKSGTSVKIECRSLDFQ |
| | FTDSAIYNLQWFRQDPGKGLT | ATTMFWYRQFPKQSLMLMAT |
| | SLLLIQSSQREQTSGRLNASLD | SNEGSKATYEQGVEKDKFLIN |
| | KSSGRSTLYIAASQPGDSATYL | HASLTLSTLTVTSAHPEDSSFYI |
| | CAGPNTGNQFYFGTGTSLTVIP | CSAPPGSPYEQYFGPGTRLTVT |
| 01CA0DP_074_ | MWGVFLLYVSMKMGGTTGQ | MGCRLLCCVVFCLLQAGPLDT |
| E12_0114 | NIDQPTEMTATEGAIVQINCTY | AVSQTPKYLVTQMGNDKSIKC |
| | QTSGFNGLFWYQQHAGEAPTF | EQNLGHDTMYWYKQDSKKFL |
| | LSYNVLDGLEEKGRFSSFLSRS | KIMFSYNNKELIINETVPNRFSP |
| | KGYSYLLLKELQMKDSASYLC | KSPDKAHLNLHINSLELGDSAV |
| | AVRGGSYIPTFGRGTSLIVHP | YFCASSQSEAGQGYGYTFGSG |
| | | TRLTVV |
| 01CA0DP_074_ | MTSIRAVFIFLWLQLDLVNGEN | MGPQLLGYVVLCLLGAGPLEA |
| E12_0160 | VEQHPSTLSVQEGDSAVIKCTY | QVTQNPRYLITVTGKKLTVTCS |
| | SDSASNYFPWYKQELGKGPQL | QNMNHEYMSWYRQDPGLGLR |
| | IIDIRSNVGEKKDQRIAVTLNKT | QIYYSMNVEVTDKGDVPEGYK |
| | AKHFSLHITETQPEDSAVYFCA | VSRKEKRNFPLILESPSPNQTSL |
| | AGGGGADGLTFGKGTHLIIQP | YFCASSDWEEGRSEQYFGPGT |
| | | RLTVT |

TABLE 1C.1
V(D)J Segments and CDR3 Sequences for TCRs isolated from healthy donors

| Clone ID | pHLA | TRA-V | TRA-J | TRB-V | TRB-D | TRB-J | αCDR3 | βCDR3 |
|---|---|---|---|---|---|---|---|---|
| 01CA0WT_066_ | HLA-A*02:01/ | TRAV24 | TRAJ33 | TRBV19 | None* | TRBJ1-2 | CAFPMDSNYQLIW | CASSLGVHGYTF |
| F03_0008 | KLVVVGACGV | | | | | | | |
| 01CA019_064_ | HLA-*02:01/ | TRAV1-2 | TRAJ37 | TRBV9 | TRBDI | TRBJ2-3 | CAVSSGNTGKLIF | CASSVAGDSLTD |
| F05_1114 | KLVVVGACGV | | | | | | | TQYF |
| 01CA0DP_064_ | HLA-A*02:01/ | TRAV38- | TRAJ28 | TRBV7-6 | None | TRBJ2-2 | CAPITGAGSYQLTF | CASSLAFQESNT |
| F04_0003 | KLVVVGACGV | 2DV8 | | | | | | GELFF |
| 01DB008_064_ | HLA-A*03:01/ | TRAV12-2 | TRAJ13 | TRBV10-2 | TRBD2 | TRBJ2-1 | CAALNSGGYQKVTF | CASSPGLNEQFF |
| G02_0013 | TTAPPLSGK | | | | | | | |
| 01DB008_064_ | HLA-A*03:01/ | TRAV12-2 | TRAJ43 | TRBV7-9 | None | TRBJ1-1 | CAVGTNDMRF | CASTSPPEAFF |
| G02_0023 | TTAPPLSGK | | | | | | | |
| 01CA018_064_ | HLA-A*03:01/ | TRAV1-2 | TRAJ20 | TRBV7-9 | None | TRBH-2 | CAVRGSNDYKLSF | CASSSDNVKNYG |
| H10_0018 | WGAVGVGK | | | | | | | YTF |
| 01CA018_064_ | HLA-A*03:01/ | TRAV12-2 | TRAJ43 | TRBV9 | None | TRBJ2-3 | CAVRNNNDMRF | CASSVGSDTQYF |
| H10_0014 | WGAVGVGK | | | | | | | |
| 01CA018_022_ | HLA-A*03:01/ | TRAV1-1 | TRAJ40 | TRBV20-1 | None | TRBJ1-1 | CAGFTSGTYKYIF | CSARVSFGVMNT |
| E04_0155 | VVVGAVGVGK | | | | | | | EAFF |
| 01CA018_022_ | HLA-A*03:01/ | TRAV12-3 | TRAJ43 | TRBV24-1 | TRBDI | TRBJ2-5 | CASYNNNDMRF | CATSGRTGGNLE |
| E04_0030 | VVVGAVGVGK | | | | | | | TQYF |
| 01CADMS_064_ | HLA-A* 11:01/ | TRAV21 | TRAJ42 | TRBV20-1 | TRBD2 | TRBJ2-7 | CASSNYGGSQGNL | CSARAAAAYEQYF |
| F09_0013 | WGAVGVGK | | | | | | IF | |
| 01CA0WT_064_ | HLA-A* 11:01/ | TRAV8-6 | TRAJ52 | TRBV6-3 | TRBDI | TRBJ1-5 | CAVSEMGGTSYGK | CASSRTGTAYQP |
| F06_0020 | WGAVGVGK | | | | | | LTF | QHF |
| 01CA018_064_ | HLA-A* 11:01/ | TRAV1-2 | TRAJ8 | TRBV5-1 | None | TRBJ1-1 | CAVDTGFQKLVF | CASSLGSPEAFF |
| G01_0167 | WGAVGVGK | | | | | | | |

*“None” indicates sequence analysis did not map to a known TRB-D gene

TABLE 1C.2
V(D)J Segments and CDR3 Sequences for TCRs isolated from healthy donors-G12C

| Clone ID | pHLA | TRA-V | TRA-J | TRB-V | TRB-D | TRB-J | TRB-C | αCDR3 | βCDR3 |
|---|---|---|---|---|---|---|---|---|---|
| 01CA019_064_ | HLA-A*02:01/ | TRAV5 | TRAJ34 | TRBV20-1 | TRBD1 | TRBJ1 | TRBC1 | CAERVNTD | CSASTGDS |
| F05_0104 | KLVVVGACGV | | | | | | | KLIF | GNTIYF |
| 01CA019_064_ | HLA-*02:01/ | TRAV14DV4 | TRAJ34 | TRBV9 | TRBD1 | TRBJ1-3 | TRBC1 | CAMRELNT | CASSQWDS |
| F05_0047 | KLVVVGACGV | | | | | | | DKLIF | SGNTIYF |
| 01CA019_064_ | HLA-A*02:01/ | TRAV14DV4 | TRAJ34 | TRBV7-2 | TRBD1 | TRBJ1-2 | TRBC1 | CAMREPRS | CASSLVSQ |
| F05_0064 | KLVVVGACGV | | | | | | | YNTDKLIF | VGYGYTF |
| 01CA019_064_ | HLA-A*02:01/ | TRAV14DV4 | TRAJ12 | TRBV9 | TRBD1 | TRBJ2-3 | TRBC2 | CAMREESS | CASSVAGD |
| F05_0005 | KLVVVGACGV | | | | | | | YKLIF | SLTDTQYF |

TABLE 1C.3
V(D)J Segments and CDR3 Sequences for TCRs isolated from healthy donors-G12V

| Clone ID | pHLA | TRA-V | TRA-J | TRB-V | TRB-D | TRB-J | TRB-C | αCDR3 | βCDR3 |
|---|---|---|---|---|---|---|---|---|---|
| 01CA018_064_ | HLA-A*11:01/ | TRAV6 | TRAJ3 | TRBV27 | None* | TRBJ2-1 | TRBC2 | CALGYSSA | CASSLVG |
| F08_0007 | VVGAVGVGK | | | | | | | SKIIF | NEQFF |
| 01CA018_064_ | HLA-A*11:01/ | TRAV29DV5 | TRAJ8 | TRBV5-1 | None | TRBJ1-1 | TRBC1 | CAANGGFQ | CASSSGR |
| F10_0022 | VVGAVGVGK | | | | | | | KLVF | TEAFF |
| 01CA018_064_ | HLA-A*11:01/ | TRAV17 | TRAJ9 | TRBVH-2 | TRBD1 | TRBJ2-7 | TRBC1 | CATDAGTG | CASSLES |
| F10_0004 | VVGAVGVGK | | | | | | | GFKTIF | SYEQYF |
| 01CA018_064_ | HLA-A*11:01/ | TRAV17 | TRAJ37 | TRBVH-2 | TRBD2 | TRBJ2-7 | TRBC2 | CATDRGSS | CASSLEI |
| F10_0005 | VVGAVGVGK | | | | | | | NTGKLIF | SYEQYF |
| 01CA018_064_ | HLA-A*11:01/ | TRAV17 | TRAJ36 | TRBVH-2 | None | TRBJ2-7 | TRBC2 | CATDSQTG | CASSLGD |
| F10 0002 | VVGAVGVGK | | | | | | | ANNLFF | TYEQYF |
| 01CA018_064_ | HLA-A*11:01/ | TRAV17 | TRAJ20 | TRBVH-2 | None | TRBJ2-7 | TRBC2 | CATDPREL | CASSLES |
| F10_0001 | VVGAVGVGK | | | | | | | SF | SYEQYF |

*“None” indicates sequence analysis did not map to a known TRB-D gene

TABLE ID
V(D)J Segments and CDR3 Sequences for TCRs isolated from TILs derived
from subjects with cancer

| Clone ID | pHLA | TRA-V | TRA-J | TRB-V | TRB-D | TRB-J | αCDR3 | βCDR3 |
|---|---|---|---|---|---|---|---|---|
| 01CA018_074_ | HLA-A*11:01/ | TRAV1-2 | TRAJ33 | TRBV4-3 | TRBD1 | TRBJ2-6 | CAVTDSN | CASSQGTG |
| E09_0061 | VVGAVGVGK | | | | | | YQLIW | SGANVLTF |
| 01CA018_074_ | HLA-A*11:01/ | TRAV21 | TRAJ49 | TRBV20-1 | None* | TRBJ2-7 | CAGPNTG | CSAPPGSP |
| E09_0035 | VVGAVGVGK | | | | | | NQFYF | YEQYF |
| 01CA0DP_074_ | HLA-A*11:01/ | TRAV1-2 | TRAJ6 | TRBV3-1 | TRBD2 | TRBH-2 | CAVRGGS | CASSQSEA |
| E12_0114 | VVVGAVGVGK | | | | | | YIPTF | GQGYGYTF |
| 01CA0DP_074_ | HLA-A*11:01/ | TRAV13-1 | TRAJ45 | TRBV27 | TRBD2 | TRBJ2-7 | CAAGGGG | CASSDWEE |
| E12_0160 | VVVGAVGVGK | | | | | | ADGLTF | GRSEQYF |

*“None” indicates sequence analysis did not map to a known TRB-D gene indicates sequence analysis did not map to a known TRB-D gene

### REFERENCES

1. Desrichard, A., Snyder, A. & Chan, T. A. Cancer Neoantigens and Applications for Immunotherapy. Clin. Cancer Res. Off J. Am. Assoc. Cancer Res. (2015). doi:10.1158/1078-0432.CCR-14-3175
2. Schumacher, T. N. & Schreiber, R. D. Neoantigens in cancer immunotherapy. Science 348, 69-74 (2015).
3. Gubin, M. M., Artyomov, M. N., Mardis, E. R. & Schreiber, R. D. Tumor neoantigens: building a framework for personalized cancer immunotherapy. J Clin. Invest. 125, 3413-3421 (2015).
4. Rizvi, N. A. et al. Cancer immunology. Mutational landscape determines sensitivity to PD-1 blockade in non-small cell lung cancer. Science 348, 124-128 (2015).
5. Snyder, A. et al. Genetic basis for clinical response to CTLA-4 blockade in melanoma. N. Engl. J. Med. 371, 2189-2199 (2014).
6. Carreno, B. M. et al. Cancer immunotherapy. A dendritic cell vaccine increases the breadth and diversity of melanoma neoantigen-specific T cells. Science 348, 803-808 (2015).
7. Tran, E. et al. Cancer immunotherapy based on mutation-specific CD4+ T cells in a patient with epithelial cancer. Science 344, 641-645 (2014).
8. Hacohen, N. & Wu, C. J.-Y. United States Patent Application: 20110293637 —COMPOSITIONS AND METHODS OF IDENTIFYING TUMOR SPECIFIC NEOANTIGENS. (A1). at <http://appft1.uspto.gov/netacgi/nph-Parser?Sect1=PTO1&Sect2=HITOFF&d=PGO1&p=1&u=/netahtml/PTO/srchnum.html&r=1 &f=G&1=50&s1=20110293637.PGNR.>
9. Lundegaard, C., Hoof, I., Lund, O. & Nielsen, M. State of the art and challenges in sequence based T-cell epitope prediction. Immunome Res. 6 Suppl 2, S3 (2010).
10. Yadav, M. et al. Predicting immunogenic tumour mutations by combining mass spectrometry and exome sequencing. Nature 515, 572-576 (2014).
11. Bassani-Sternberg, M., Pletscher-Frankild, S., Jensen, L. J. & Mann, M. Mass spectrometry of human leukocyte antigen class I peptidomes reveals strong effects of protein abundance and turnover on antigen presentation. Mol. Cell. Proteomics MCP 14, 658-673 (2015).
12. Van Allen, E. M. et al. Genomic correlates of response to CTLA-4 blockade in metastatic melanoma. Science 350, 207-211 (2015).
13. Yoshida, K. & Ogawa, S. Splicing factor mutations and cancer. Wiley Interdiscip. Rev. RNA 5, 445-459 (2014).
14. Cancer Genome Atlas Research Network. Comprehensive molecular profiling of lung adenocarcinoma. Nature 511, 543-550 (2014).
15. Rajasagi, M. et al. Systematic identification of personal tumor-specific neoantigens in chronic lymphocytic leukemia. Blood 124, 453-462 (2014).
16. Downing, S. R. et al. U.S. patent application Ser. No. 01/202,08706 —OPTIMIZATION OF MULTIGENE ANALYSIS OF TUMOR SAMPLES. (A1). at <http://appft1.uspto.gov/netacgi/nph-Parser?Sect1=PTO1&Sect2=HITOFF&d=PGO1&p=1&u=/netahtml/PTO/srchnum.html&r=1 &f=G&1=50&s1=20120208706.PGNR.>
17. Target Capture for NextGen Sequencing—IDT. at <http://www.idtdna.com/pages/products/nextgen/target-capture>
18. Shukla, S. A. et al. Comprehensive analysis of cancer-associated somatic mutations in class I HLA genes. Nat. Biotechnol. 33, 1152-1158 (2015).
19. Cieslik, M. et al. The use of exome capture RNA-seq for highly degraded RNA with application to clinical cancer sequencing. Genome Res. 25, 1372-1381 (2015).
20. Bodini, M. et al. The hidden genomic landscape of acute myeloid leukemia: subclonal structure revealed by undetected mutations. Blood 125, 600-605 (2015).
21. Saunders, C. T. et al. Strelka: accurate somatic small-variant calling from sequenced tumor-normal sample pairs. Bioinforma. Oxf. Engl. 28, 1811-1817 (2012).
22. Cibulskis, K. et al. Sensitive detection of somatic point mutations in impure and heterogeneous cancer samples. Nat. Biotechnol. 31, 213-219 (2013).
23. Wilkerson, M. D. et al. Integrated RNA and DNA sequencing improves mutation detection in low purity tumors. Nucleic Acids Res. 42, e107 (2014).
24. Mose, L. E., Wilkerson, M. D., Hayes, D. N., Perou, C. M. & Parker, J. S. ABRA: improved coding indel detection via assembly-based realignment. Bioinforma. Oxf Engl. 30, 2813-2815 (2014).
25. Ye, K., Schulz, M. H., Long, Q., Apweiler, R. & Ning, Z. Pindel: a pattern growth approach to detect break points of large deletions and medium sized insertions from paired-end short reads. Bioinforma. Oxf Engl. 25, 2865-2871 (2009).
26. Lam, H. Y. K. et al. Nucleotide-resolution analysis of structural variants using BreakSeq and a breakpoint library. Nat. Biotechnol. 28, 47-55 (2010).
27. Frampton, G. M. et al. Development and validation of a clinical cancer genomic profiling test based on massively parallel DNA sequencing. Nat. Biotechnol. 31, 1023-1031 (2013).
28. Boegel, S. et al. HLA typing from RNA-Seq sequence reads. Genome Med. 4, 102 (2012).
29. Liu, C. et al. ATHLATES: accurate typing of human leukocyte antigen through exome sequencing. Nucleic Acids Res. 41, e142 (2013).
30. Mayor, N. P. et al. HLA Typing for the Next Generation. PloS One 10, e0127153 (2015).
31. Roy, C. K., Olson, S., Graveley, B. R., Zamore, P. D. & Moore, M. J. Assessing long-distance RNA sequence connectivity via RNA-templated DNA-DNA ligation. eLife 4, (2015).
32. Song, L. & Florea, L. CLASS: constrained transcript assembly of RNA-seq reads. BMC Bioinformatics 14 Suppl 5, S14 (2013).
33. Maretty, L., Sibbesen, J. A. & Krogh, A. Bayesian transcriptome assembly. Genome Biol. 15, 501 (2014).
34. Pertea, M. et al. StringTie enables improved reconstruction of a transcriptome from RNA-seq reads. Nat. Biotechnol. 33, 290-295 (2015).
35. Roberts, A., Pimentel, H., Trapnell, C. & Pachter, L. Identification of novel transcripts in annotated genomes using RNA-Seq. Bioinforma. Oxf Engl. (2011). doi:10.1093/bioinformatics/btr355
36. Vitting-Seerup, K., Porse, B. T., Sandelin, A. & Waage, J. spliceR: an R package for classification of alternative splicing and prediction of coding potential from RNA-seq data. BMC Bioinformatics 15, 81 (2014).
37. Rivas, M. A. et al. Human genomics. Effect of predicted protein-truncating genetic variants on the human transcriptome. Science 348, 666-669 (2015).
38. Skelly, D. A., Johansson, M., Madeoy, J., Wakefield, J. & Akey, J. M. A powerful and flexible statistical framework for testing hypotheses of allele-specific gene expression from RNA-seq data. Genome Res. 21, 1728-1737 (2011).
39. Anders, S., Pyl, P. T. & Huber, W. HTSeq—a Python framework to work with high-throughput sequencing data. Bioinforma. Oxf Engl. 31, 166-169 (2015).
40. Furney, S. J. et al. SF3B1 mutations are associated with alternative splicing in uveal melanoma. Cancer Discov. (2013). doi:10.1158/2159-8290.CD-13-0330
41. Zhou, Q. et al. A chemical genetics approach for the functional assessment of novel cancer genes. Cancer Res. (2015). doi:10.1158/0008-5472.CAN-14-2930
42. Maguire, S. L. et al. SF3B1 mutations constitute a novel therapeutic target in breast cancer. J. Pathol. 235, 571-580 (2015).
43. Carithers, L. J. et al. A Novel Approach to High-Quality Postmortem Tissue Procurement: The GTEx Project. Biopreservation Biobanking 13, 311-319 (2015).
44. Xu, G. et al. RNA CoMPASS: a dual approach for pathogen and host transcriptome analysis of RNA-seq datasets. PloS One 9, e89445 (2014).
45. Andreatta, M. & Nielsen, M. Gapped sequence alignment using artificial neural networks: application to the MHC class I system. Bioinforma. Oxf. Engl. (2015). doi:10.1093/bioinformatics/btv639
46. Jorgensen, K. W., Rasmussen, M., Buus, S. & Nielsen, M. NetMHCstab—predicting stability of peptide-MHC-I complexes; impacts for cytotoxic T lymphocyte epitope discovery. Immunology 141, 18-26 (2014).
47. Larsen, M. V. et al. An integrative approach to CTL epitope prediction: a combined algorithm integrating MHC class I binding, TAP transport efficiency, and proteasomal cleavage predictions. Eur. J. Immunol. 35, 2295-2303 (2005).
48. Nielsen, M., Lundegaard, C., Lund, O. & Keşmir, C. The role of the proteasome in generating cytotoxic T-cell epitopes: insights obtained from improved predictions of proteasomal cleavage. Immunogenetics 57, 33-41 (2005).
49. Boisvert, F.-M. et al. A Quantitative Spatial Proteomics Analysis of Proteome Turnover in Human Cells. Mol. Cell. Proteomics 11, M111.011429-M111.011429 (2012).
50. Duan, F. et al. Genomic and bioinformatic profiling of mutational neoepitopes reveals new rules to predict anticancer immunogenicity. J. Exp. Med. 211, 2231-2248 (2014).
51. Janeway's Immunobiology: 9780815345312: Medicine & Health Science Books @Amazon.com. at <http://www.amazon.com/Janeways-Immunobiology-Kenneth-Murphy/dp/0815345313>
52. Calis, J. J. A. et al. Properties of MHC Class I Presented Peptides That Enhance Immunogenicity. PLoS Comput. Biol. 9, e1003266 (2013).
53. Zhang, J. et al. Intratumor heterogeneity in localized lung adenocarcinomas delineated by multiregion sequencing. Science 346, 256-259 (2014)
54. Walter, M. J. et al. Clonal architecture of secondary acute myeloid leukemia. N. Engl. J. Med. 366, 1090-1098 (2012).
55. Hunt D F, Henderson R A, Shabanowitz J, Sakaguchi K, Michel H, Sevilir N, Cox A L, Appella E, Engelhard V H. Characterization of peptides bound to the class I MHC molecule HLA-A2.1 by mass spectrometry. Science 1992. 255: 1261-1263.
56. Zarling A L, Polefrone J M, Evans A M, Mikesh L M, Shabanowitz J, Lewis S T, Engelhard V H, Hunt D F. Identification of class I MHC-associated phosphopeptides as targets for cancer immunotherapy. Proc Natl Acad Sci USA. 2006 Oct. 3; 103(40):14889-94.
57. Bassani-Sternberg M, Pletscher-Frankild S, Jensen L J, Mann M. Mass spectrometry of human leukocyte antigen class I peptidomes reveals strong effects of protein abundance and turnover on antigen presentation. Mol Cell Proteomics. 2015 March; 14(3):658-73. doi: 10.1074/mcp.M 114.042812.
58. Abelin J G, Trantham P D, Penny S A, Patterson A M, Ward S T, Hildebrand W H, Cobbold M, Bai D L, Shabanowitz J, Hunt D F. Complementary IMAC enrichment methods for HLA-associated phosphopeptide identification by mass spectrometry. Nat Protoc. 2015 September; 10(9):1308-18. doi: 10.1038/nprot. 2015.086. Epub 2015 Aug. 6
59. Barnstable C J, Bodmer W F, Brown G, Galfre G, Milstein C, Williams A F, Ziegler A. Production of monoclonal antibodies to group A erythrocytes, HLA and other human cell surface antigens-new tools for genetic analysis. Cell. 1978 May; 14(1):9-20.
60. Goldman J M, Hibbin J, Kearney L, Orchard K, Th'ng K H. HLA-D R monoclonal antibodies inhibit the proliferation of normal and chronic granulocytic leukaemia myeloid progenitor cells. Br J Haematol. 1982 November; 52(3):411-20.
61. Eng J K, Jahan T A, Hoopmann M R. Comet: an open-source MS/MS sequence database search tool. Proteomics. 2013 January; 13(1):22-4. doi: 10.1002/pmic. 201200439. Epub 2012 Dec. 4.
62. Eng J K, Hoopmann M R, Jahan T A, Egertson J D, Noble W S, MacCoss M J. A deeper look into Comet—implementation and features. J Am Soc Mass Spectrom. 2015 November; 26(11):1865-74. doi: 10.1007/s13361-015-1179-x. Epub 2015 Jun. 27.
63. Lukas Käll, Jesse Canterbury, Jason Weston, William Stafford Noble and Michael J. MacCoss. Semi-supervised learning for peptide identification from shotgun proteomics datasets. Nature Methods 4:923-925, November 2007
64. Lukas Käll, John D. Storey, Michael J. MacCoss and William Stafford Noble. Assigning confidence measures to peptides identified by tandem mass spectrometry. Journal of Proteome Research, 7(1):29-34, January 2008
65. Lukas Käll, John D. Storey and William Stafford Noble. Nonparametric estimation of posterior error probabilities associated with peptides identified by tandem mass spectrometry. Bioinformatics, 24(16):i42-i48, August 2008
66. Kinney R M, B J Johnson, V L Brown, D W Trent. Nucleotide Sequence of the 26 S mRNA of the Virulent Trinidad Donkey Strain of Venezuelan Equine Encephalitis Virus and Deduced Sequence of the Encoded Structural Proteins. Virology 152 (2), 400-413. 1986 Jul. 30.
67. Jill E Slansky, Frédérique M Rattis, Lisa F Boyd, Tarek Fahmy, Elizabeth M Jaffee, Jonathan P Schneck, David H Margulies, Drew M Pardoll. Enhanced Antigen-Specific Antitumor Immunity with Altered Peptide Ligands that Stabilize the MHC-Peptide-TCR Complex. Immunity, Volume 13, Issue 4, 1 Oct. 2000, Pages 529-538.
68. A Y Huang, P H Gulden, A S Woods, M C Thomas, C D Tong, W Wang, V H Engelhard, G Pasternack, R Cotter, D Hunt, D M Pardoll, and E M Jaffee. The immunodominant major histocompatibility complex class I-restricted antigen of a murine colon tumor derives from an endogenous retroviral gene product. Proc Natl Acad Sci USA; 93(18): 9730-9735, 1996 Sep. 3.
69. JOHNSON, BARBARA J. B., RICHARD M. KINNEY, CRYSTLE L. KOST AND DENNIS W. TRENT. Molecular Determinants of Alphavirus Neurovirulence: Nucleotide and Deduced Protein Sequence Changes during Attenuation of Venezuelan Equine Encephalitis Virus. J Gen Virol 67:1951-1960, 1986.
70. Aarnoudse, C. A., Krüse, M., Konopitzky, R., Brouwenstijn, N., and Schrier, P. I. (2002). TCR reconstitution in Jurkat reporter cells facilitates the identification of novel tumor antigens by cDNA expression cloning. Int J Cancer 99, 7-13.
71. Alexander, J., Sidney, J., Southwood, S., Ruppert, J., Oseroff, C., Maewal, A., Snoke, K., Serra, H. M., Kubo, R. T., and Sette, A. (1994). Development of high potency universal DR-restricted helper epitopes by modification of high affinity DR-blocking peptides. Immunity 1, 751-761.
72. Banu, N., Chia, A., Ho, Z. Z., Garcia, A. T., Paravasivam, K., Grotenbreg, G. M., Bertoletti, A., and Gehring, A. J. (2014). Building and optimizing a virus-specific T cell receptor library for targeted immunotherapy in viral infections. Scientific Reports 4, 4166.
73. Cornet, S., Miconnet, I., Menez, J., Lemonnier, F., and Kosmatopoulos, K. (2006). Optimal organization of a polypeptide-based candidate cancer vaccine composed of cryptic tumor peptides with enhanced immunogenicity. Vaccine 24, 2102-2109.
74. Depla, E., van der Aa, A., Livingston, B. D., Crimi, C., Allosery, K., de Brabandere, V., Krakover, J., Murthy, S., Huang, M., Power, S., et al. (2008). Rational design of a multiepitope vaccine encoding T-lymphocyte epitopes for treatment of chronic hepatitis B virus infections. Journal of Virology 82, 435-450.
75. Ishioka, G. Y., Fikes, J., Hermanson, G., Livingston, B., Crimi, C., Qin, M., del Guercio, M. F., Oseroff, C., Dahlberg, C., Alexander, J., et al. (1999). Utilization of MHC class I transgenic mice for development of minigene DNA vaccines encoding multiple HLA-restricted CTL epitopes. J Immunol 162, 3915-3925.
76. Janetzki, S., Price, L., Schroeder, H., Britten, C. M., Welters, M. J. P., and Hoos, A. (2015). Guidelines for the automated evaluation of Elispot assays. Nat Protoc 10, 1098-1115.
77. Lyons, G. E., Moore, T., Brasic, N., Li, M., Roszkowski, J. J., and Nishimura, M. I. (2006). Influence of human CD8 on antigen recognition by T-cell receptor-transduced cells. Cancer Res 66, 11455-11461.
78. Nagai, K., Ochi, T., Fujiwara, H., An, J., Shirakata, T., Mineno, J., Kuzushima, K., Shiku, H., Melenhorst, J. J., Gostick, E., et al. (2012). Aurora kinase A-specific T-cell receptor gene transfer redirects T lymphocytes to display effective antileukemia reactivity. Blood 119, 368-376.
79. Panina-Bordignon, P., Tan, A., Termijtelen, A., Demotz, S., Corradin, G., and Lanzavecchia, A. (1989). Universally immunogenic T cell epitopes: promiscuous binding to human MHC class II and promiscuous recognition by T cells. Eur J Immunol 19, 2237-2242.
80. Vitiello, A., Marchesini, D., Furze, J., Sherman, L. A., and Chesnut, R. W. (1991). Analysis of the HLA-restricted influenza-specific cytotoxic T lymphocyte response in transgenic mice carrying a chimeric human-mouse class I major histocompatibility complex. J Exp Med 173, 1007-1015.
81. Yachi, P. P., Ampudia, J., Zal, T., and Gascoigne, N. R. J. (2006). Altered peptide ligands induce delayed CD8-T cell receptor interaction—a role for CD8 in distinguishing antigen quality. Immunity 25, 203-211.
82. Pushko P, Parker M, Ludwig G V, Davis N L, Johnston R E, Smith J F. Replicon-helper systems from attenuated Venezuelan equine encephalitis virus: expression of heterologous genes in vitro and immunization against heterologous pathogens in vivo. Virology. 1997 Dec. 22; 239(2):389-401.
83. Strauss, J H and E G Strauss. The alphaviruses: gene expression, replication, and evolution. Microbiol Rev. 1994 September; 58(3): 491-562.
84. Rhême C, Ehrengruber M U, Grandgirard D. Alphaviral cytotoxicity and its implication in vector development. Exp Physiol. 2005 January; 90(1):45-52. Epub 2004 Nov. 12.
85. Riley, Michael K. I I, and Wilfred Vermerris. Recent Advances in Nanomaterials for Gene Delivery—A Review. Nanomaterials 2017, 7(5), 94.
86. Frolov I, Hardy R, Rice C M. Cis-acting RNA elements at the 5′ end of Sindbis virus genome RNA regulate minus- and plus-strand RNA synthesis. RNA. 2001 November; 7(11):1638-51.
87. Jose J, Snyder J E, Kuhn R J. A structural and functional perspective of alphavirus replication and assembly. Future Microbiol. 2009 September; 4(7):837-56.
88. Bo Li and C. olin N. Dewey. RSEM: accurate transcript quantification from RNA-Seq data with or without a reference genome. BMC Bioinformatics, 12:323, August 2011
89. Hillary Pearson, Tariq Daouda, Diana Paola Granados, Chantal Durette, Eric Bonneil, Mathieu Courcelles, Anja Rodenbrock, Jean-Philippe Laverdure, Caroline Côté, Sylvie Mader, Sébastien Lemieux, Pierre Thibault, and Claude Perreault. MHC class I-associated peptides derive from selective regions of the human genome. The Journal of Clinical Investigation, 2016,
90. Juliane Liepe, Fabio Marino, John Sidney, Anita Jeko, Daniel E. Bunting, Alessandro Sette, Peter M. Kloetzel, Michael P. H. Stumpf, Albert J. R. Heck, Michele Mishto. A large fraction of HLA class I ligands are proteasome-generated spliced peptides. Science, 21, October 2016.
91. Mommen G P., Marino, F., Meiring H D., Poelen, M C., van Gaans-van den Brink, J A., Mohammed S., Heck A J., and van Els C A. Sampling From the Proteome to the Human Leukocyte Antigen-D R (HLA-D R) Ligandome Proceeds Via High Specificity. Mol Cell Proteomics 15(4): 1412-1423, April 2016.
92. Sebastian Kreiter, Mathias Vormehr, Niels van de Roemer, Mustafa Diken, Martin Löwer, Jan Diekmann, Sebastian Boegel, Barbara Schrörs, Fulvia Vascotto, John C. Castle, Arbel D. Tadmor, Stephen P. Schoenberger, Christoph Huber, Özlem Túreci, and Ugur Sahin. Mutant MHC class I I epitopes drive therapeutic immune responses to caner. Nature 520, 692-696, April 2015.
93. Tran E., Turcotte S., Gros A., Robbins P. F., Lu Y. C., Dudley M E., Wunderlich J R., Somerville R. P., Hogan K., Hinrichs C. S., Parkhurst M. R., Yang J. C., Rosenberg S. A. Cancer immunotherapy based on mutation-specific CD4+ T cells in a patient with epithelial cancer. Science 344(6184) 641-645, May 2014.
94. Andreatta M., Karosiene E., Rasmussen M., Stryhn A., Buus S., Nielsen M. Accurate pan-specific prediction of peptide-MHC class II binding affinity with improved binding core identification. Immunogenetics 67(11-12) 641-650, November 2015.
95. Nielsen, M., Lund, O. N N-align. An artificial neural network-based alignment algorithm for MHC class I I peptide binding prediction. BMC Bioinformatics 10:296, September 2009.
96. Nielsen, M., Lundegaard, C., Lund, O. Prediction of MHC class II binding affinity using SMM-align, a novel stabilization matrix alignment method. BMC Bioinformatics 8:238, July 2007.
97. Zhang, J., et al. PEAKS D B: de novo sequencing assisted database search for sensitive and accurate peptide identification. Molecular & Cellular Proteomics. 11(4):1-8. Jan. 2, 2012.
98. Jensen, Kamilla Kjaergaard, et al. “Improved Methods for Prediting Peptide Binding Affinity to MHC Class I I Molecules.” Immunology, 2018, doi:10.1111/imm. 12889.
99. Carter, S. L., Cibulskis, K., Helman, E., McKenna, A., Shen, H., Zack, T., Laird, P. W., Onofrio, R. C., Winckler, W., Weir, B. A., et al. (2012). Absolute quantification of somatic DNA alterations in human cancer. Nat. Biotechnol. 30, 413-421
100. McGranahan, N., Rosenthal, R., Hiley, C. T., Rowan, A. J., Watkins, T. B. K., Wilson, G. A., Birkbak, N. J., Veeriah, S., Van Loo, P., Herrero, J., et al. (2017). Allele-Specific HLA Loss and Immune Escape in Lung Cancer Evolution. Cell 171, 1259-1271.ell.
101. Shukla, S. A., Rooney, M. S., Rajasagi, M., Tiao, G., Dixon, P. M., Lawrence, M. S., Stevens, J., Lane, W. J., Dellagatta, J. L., Steelman, S., et al. (2015). Comprehensive analysis of cancer-associated somatic mutations in class I HLA genes. Nat. Biotechnol. 33, 1152-1158.
102. Van Loo, P., Nordgard, S. H., Lingjorde, O. C., Russnes, H. G., Rye, I. H., Sun, W., Weigman, V. J., Marynen, P., Zetterberg, A., Naume, B., et al. (2010). Allele-specific copy number analysis of tumors. Proc. Natl. Acad. Sci. U.S.A 107, 16910-16915.
103. Van Loo, P., Nordgard, S. H., Lingjærde, O. C., Russnes, H. G., Rye, I. H., Sun, W., Weigman, V. J., Marynen, P., Zetterberg, A., Naume, B., et al. (2010). Allele-specific copy number analysis of tumors. Proc. Natl. Acad. Sci. U.S.A 107, 16910-16915.

1. An antigen binding protein (ABP) that specifically binds to an HLA-PEPTIDE antigen comprising an HLA-restricted peptide complexed with an HLA Class I molecule, wherein the HLA-restricted peptide is located in the peptide binding groove of an α1/α2 heterodimer portion of the HLA Class I molecule, wherein the HLA Class I molecule and the HLA-restricted peptide are each selected from an HLA-PEPTIDE antigen as described in any one of SEQ ID NOs:10,755 to 29,364, and wherein the ABP comprises a T cell receptor (TCR) or antigen-binding fragment thereof.

2-3. (canceled)

4. The ABP of claim 1, wherein the HLA-PEPTIDE antigen is selected from the group consisting of:
a. a RAS_G12D MHC Class I antigen comprising HLA-A*11:01 and the restricted peptide VVVGADGVGK;
b. a RAS_G12V MHC Class I antigen comprising HLA-A*11:01 and the restricted peptide VVVGAVGVGK;
c. a RAS_G12C MHC Class I antigen comprising HLA-A*02:01 and the restricted peptide KLVVVGACGV;
d. a CTNNB1_S45P MHC Class I antigen comprising HLA-A*03:01 and the restricted peptide TTAPPLSGK;
e. a RAS_G12D MHC Class I antigen comprising HLA-A*11:01 and the restricted peptide VVGADGVGK;
f. a RAS_G12V MHC Class I antigen comprising HLA-A*11:01 and the restricted peptide VVGAVGVGK;
g. a RAS_G12V MHC Class I antigen comprising HLA-C*01:02 and the restricted peptide AVGVGKSAL;
h. a RAS_G12V MHC Class I antigen comprising HLA-A*03:01 and the restricted peptide VVVGAVGVGK;
i. a TP53_K132N MHC Class I antigen comprising HLA-A*24:02 and the restricted peptide TYSPALNNMF;
j. a CTNNB1_S37Y MHC Class I antigen comprising HLA-A*02:01 and the restricted peptide YLDSGIHYGA;
k. a RAS_G12C MHC Class I antigen comprising HLA-A*03:01 and the restricted peptide VVVGACGVGK;
l. a RAS_G12C MHC Class I antigen comprising HLA-A*11:01 and the restricted peptide VVVGACGVGK;
m. a RAS_G12D MHC Class I antigen comprising HLA-A*03:01 and the restricted peptide VVVGADGVGK;
n. a RAS_Q61H MHC Class I antigen comprising HLA-A*01:01 and the restricted peptide ILDTAGHEEY; and
o. a TP53_R213L MHC Class I antigen comprising A*02:01 and the restricted peptide YLDDRNTFL.

5-7. (canceled)

8. The ABP of claim 1, wherein the HLA-restricted peptide comprises a RAS G12 mutation, optionally wherein the G12 mutation is a G12C, a G12D, a G12V, or a G12A mutation.

9-50. (canceled)

51. An engineered cell expressing a receptor comprising the antigen binding protein of claim 1.

52. The engineered cell of claim 51, wherein the engineered cell is a T cell, optionally wherein the T cell is selected from the group consisting of: a naive T (TN) cell, an effector T cell (TEFF), a memory T cell, a stem cell memory T cell (TSCM), a central memory T cell (TCM), an effector memory T cell (TEM), a terminally differentiated effector memory T cell, a tumor-infiltrating lymphocyte (TIL), an immature T cell, a mature T cell, a helper T cell, a cytotoxic T cell (CTL), a mucosa-associated invariant T (MALT) cell, a regulatory T cell (Treg), a TH1 cell, a TH2 cell, a TH3 cell, a TH17 cell, a TH9 cell, a TH22 cell, a follicular helper T cell, an natural killer T cell (NKT), an alpha-beta T cell, and a gamma-delta T cell.

53-55. (canceled)

56. The engineered cell of claim 51, wherein the engineered cell is an autologous cell of a subject.

57. The engineered cell of claim 56, wherein the subject is known or suspected to have cancer.

58-61. (canceled)

62. The engineered cell of claim 56, wherein the ABP comprises a T cell receptor (TCR) or an antigen-binding portion thereof, and wherein a polynucleotide encoding the T cell receptor (TCR) or antigen-binding portion thereof is inserted in an endogenous TCR locus.

63. (canceled)

64. An isolated polynucleotide or set of polynucleotides encoding the ABP of claim 1 or an antigen-binding portion thereof.

65-70. (canceled)

71. A pharmaceutical composition comprising the antigen binding protein of claim 1 and a pharmaceutically acceptable excipient.

72. (canceled)

73. A method of stimulating an immune response in a subject, comprising administering to the subject an antigen binding protein (ABP) that specifically binds to an HLA-PEPTIDE antigen comprising an HLA-restricted peptide complexed with an HLA Class I molecule, wherein the HLA-restricted peptide is located in the peptide binding groove of an α1/α2 heterodimer portion of the HLA Class I molecule, wherein the HLA Class I molecule and the HLA-restricted peptide are each selected from an HLA-PEPTIDE antigen as described in any one of SEQ ID NOs:10,755 to 29,364, and wherein the ABP comprises a T cell receptor (TCR) or antigen-binding fragment thereof, optionally wherein the subject has cancer, optionally wherein the cancer is selected from a solid tumor and a hematological tumor.

74-75. (canceled)

76. The method of claim 73, wherein the cancer expresses or is predicted to express an HLA-PEPTIDE antigen or HLA Class I molecule as described in any one of SEQ ID NOs:10,755 to 29,364, and wherein the ABP binds to the HLA-PEPTIDE antigen, optionally wherein the cancer expresses or is predicted to express an HLA-PEPTIDE antigen comprising an HLA-restricted peptide complexed with an HLA Class I molecule, wherein the HLA-restricted peptide is located in the peptide binding groove of an α1/α2 heterodimer portion of the HLA Class I molecule, wherein the HLA Class I molecule and the HLA-restricted peptide are each selected from an HLA-PEPTIDE antigen as described in any one of SEQ ID NOs:10,755 to 29,364, and wherein the ABP binds to the HLA-PEPTIDE antigen.

77-154. (canceled)

155. The ABP of claim 1, ABP comprises an alpha-CDR3 amino acid sequence and corresponding beta-CDR3 amino acid sequence selected from the group consisting of the sequences shown in Tables 1C. 2 and 1C. 3.

156. The ABP of claim 155, wherein the ABP further comprises an alpha variable (“V”) segment, an alpha joining (“J”) segment, a beta variable (“V”) segment, a beta joining (“J”) segment, optionally a beta diversity (“D”) segment, and optionally a beta constant region selected from the group consisting of the regions shown in Tables 1C. 2 and 1C. 3 corresponding to the alpha-CDR3 amino acid sequence and corresponding beta-CDR3 amino acid sequence.

157. The ABP of claim 155, wherein the ABP comprises an alpha variable region and corresponding beta variable region comprising the amino acid sequences selected from the sequences shown in Tables 1A. 2 and 1A. 3 corresponding to the alpha-CDR3 amino acid sequence and corresponding beta-CDR3 amino acid sequence.

158. An antigen binding protein (ABP) that specifically binds to an HLA-PEPTIDE antigen comprising an HLA-restricted RAS peptide complexed with an HLA Class I molecule, wherein the HLA-restricted peptide is located in the peptide binding groove of an α1/α2 heterodimer portion of the HLA Class I molecule, wherein the HLA-restricted RAS peptide comprises at least one alteration that makes HLA-restricted RAS peptide sequence distinct from the corresponding peptide sequence of a wild-type RAS peptide, and wherein the ABP comprises an alpha-CDR3 amino acid sequence and corresponding beta-CDR3 amino acid sequence selected from the group consisting of the sequences shown in Tables 1C. 2 and 1C. 3.

159. (canceled)

160. The ABP of claim 158, wherein the HLA-PEPTIDE antigen is (a) a RAS_G12C MHC Class I antigen comprising HLA-A*02:01 and the restricted peptide KLVVVGACGV, or (b) a RAS G12V MHC Class I antigen comprising HLA-A*11:01 and the restricted peptide VVGAVGVGK.

161. The ABP of claim 160, wherein the ABP comprises an alpha-CDR3 amino acid sequence and corresponding beta-CDR3 amino acid sequence selected from the group consisting of the sequences shown in Table 1C. 2 or Table 1C. 3.

162. The ABP of claim 161, wherein the ABP further comprises an alpha variable (“V”) segment, an alpha joining (“J”) segment, a beta variable (“V”) segment, a beta joining (“J”) segment, optionally a beta diversity (“D”) segment, and optionally a beta constant region selected from the group consisting of the regions shown in Table 1C. 2 or Table 1C. 3 corresponding to the alpha-CDR3 amino acid sequence and corresponding beta-CDR3 amino acid sequence.

163. The ABP of claim 161, wherein the ABP comprises an alpha variable region and corresponding beta variable region comprising the amino acid sequences selected from the sequences shown in Table 1A. 2 or Table 1A. 3 corresponding to the alpha-CDR3 amino acid sequence and corresponding beta-CDR3 amino acid sequence.

164-167. (canceled)
